(19) United States
(12) Patent Application Publication
(10) Pub. No.: US 2025/0367314 A1
(43) Pub. Date: Dec. 4, 2025

(54) LINKER-PAYLOAD, AN ANTIBODY-DRUG CONJUGATE MADE THEREBY AND USES THEREOF

(71) Applicants: Du-Shieng Chien, Guilford, CT (US); TAIRX, INC., Taipei (TW)

(72) Inventors: Du-Shieng CHIEN, Guilford, CT (US); Yi-Wen Chu, Taipei (TW); Yen-Ling Chen, Taipei (TW); Chia-Chung Lee, Taipei (TW); Yuan-Ming Lee, Taipei (TW); Sheng-Chu Kuo, Taipei (TW)

(73) Assignees: Du-Shieng Chien, Guilford, CT (US); TAIRX, INC., Taipei (TW)

(21) Appl. No.: 19/220,777

(22) Filed: May 28, 2025

Related U.S. Application Data
(60) Provisional application No. 63/654,474, filed on May 31, 2024.

(51) Int. Cl. A61K 47/68

(52) U.S. Cl. CPC A61K 47/6851; A61K 47/6845; A61K 47/6849; A61K 47/6853; A61K 47/6869; A61K 47/6889

(57) ABSTRACT

The present disclosure provides a linker-payload being effective in treating a cancer, wherein the linker-payload is made by connecting a linking group to a specific site of an aryl-quinolin derivative having a structure of the following Formula (I):

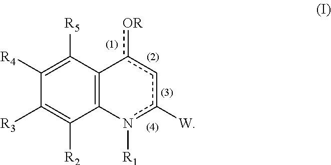

The present disclosure also relates to an antibody-drug conjugate made by further conjugating the aforementioned linker-payload to an antibody, and uses of the linker-payload and antibody-drug conjugate.

### CROSS-REFERENCE TO RELATED APPLICATIONS

This application claims priority to U.S. Provisional Patent Application No. 63/654,474, filed on May 31, 2024. The entirety of the above-mentioned patent application is hereby incorporated by reference herein and made a part of this specification.

### BACKGROUND

### 1. Technical Field

The present disclosure relates to a linker-payload composed by a linking group and an aryl-quinolin derivative, especially to a linker-payload made by connecting the linking group to a specific site of the aryl-quinolin derivative. The present disclosure also relates to an antibody-drug conjugate made by further conjugating the aforementioned linker-payload to an antibody and uses of the linker-payload and antibody-drug conjugate.

### 2. Description of Associated Art

Currently, many procedures and drugs are available for treating cancer, and most patients suffering from cancer are treated with a combination therapy, such as surgery with chemotherapy and/or radiation therapy. Other treatments such as immunotherapy, targeted therapy and hormone therapy are also available in recent years.

However, cancer is still a leading cause of death worldwide. The reasons for failing to achieve an effective treatment by using the conventional drugs are complicated, but may due to the genetic polymorphisms of patients, non-specificity of drug action, drug resistance caused by efflux through transporters, narrow therapeutic window, etc.

To overcome the above defects, the industries is still dedicated to develop new drugs that can effectively treat cancer. One of the most successful strategies of developing anti-cancer drugs is to provide an antibody-drug conjugate (ADC) that can target to specific tissues. Antibody-drug conjugate is typically composed of a monoclonal antibody (mAbs) covalently attached to a cytotoxic drug via a chemical linker, and it can release the drug (payload) at the target site. To design an antibody-drug conjugate that can release effective drugs therefrom, the specific site of drug for linkers to connected thereto should be carefully selected.

The applicant has developed a series of aryl-quinolin derivatives that is effective in treating many kinds of cancer by inhibiting the function of mitochondrial chaperon protein TRAP1 (also known as HSP75), which promotes mitochondrial apoptosis and inhibits vasculogenic mimicry (VM). One of those derivatives is undergoing clinical phase 2 trials as an oral anti-cancer drug. The preparation method for those aryl-quinolin derivatives can be seen in U.S. Pat. No. 8,524,740 B2 and U.S. Pat. No. 9,717,721 B2. However, the way for developing the aryl-quinolin derivatives to be a linker-payload with linkers or making an antibody-drug conjugate therefrom remains unknown.

### SUMMARY

Given the above, the present disclosure provides a linker-payload and an antibody-drug conjugate made by connecting the aryl-quinolin derivatives to a linking group at a specific site. The linker-payload or an antibody-drug conjugate thus obtained can further improve the concentration of the drug at the target tissue so as to broaden the therapeutic window, and thus become a potential drug for the treatment of cancer.

The present disclosure provides a linker-payload having a structure of L-(D)ₚ,
wherein L is a linking group;
p is an integer selected from 1 to 3;
D is a drug having a structure of the following Formula (I):

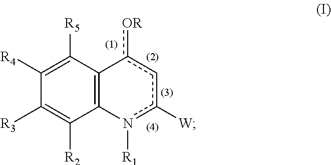

wherein the linking group (L) is connected to the drug (D) at R₅;
R is absent or present, and wherein R, if present, is hydrogen, P(═O)(OH)₂, P(═O)(O(C₁-C₁₈)alkylene(C₆-C₂₀)aryl)₂, P(═O)(OH)(OM), P(═O)(OM)₂, P═O(O₂M), S(═O)(OH)₂, S(═O)(O(C₁-C₁₈)alkylene(C₆-C₂₀)aryl)₂, S(═O)(OH)(OM), S(═O)(OM)₂, wherein Mis a monovalent or divalent metal ion, or alkylammonium ion;
R₁ is absent or present, and wherein R₁, if present, is hydrogen;
R₂, R₃ and R₄ independently are H, F, Cl, Br, (CH₂)ₙCH₃, (CH₂)ₙOH, O(CH₂)ₙCH₃, O(CH₂)ₙOH, O(CH₂)ₙNR₈R₉, (CH₂)ₙSH, S(CH₂)ₙCH₃, S(CH₂)ₙSH, S(CH₂)ₙNR₈R₉, (CH₂)ₙNR₈R₉, (CH₂)ₙN, or R₃ and R₄ together is —O(CH₂)ₙO— or —S(CH₂)ₙS—;
R₅ is O;
each of bonds (1) to (4) independently represents a single bond or a double bond, provided that when bond (1) is a single bond, R is present, R₁ is absent, each of bonds (2) and (4) is a double bond, and bond (3) is a single bond; and when bond (1) is a double bond, R is absent, R₁ is present, each of bonds (2) and (4) is a single bond, and bond (3) is a double bond;
W is an aromatic group selected from the group consisting of naphthyl, quinolinyl, benzofuranyl, benzothiophenyl, anthracenyl, and substituted phenyl of formula (Y):

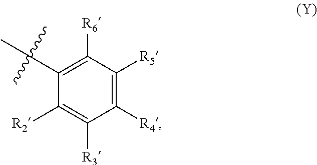

and wherein:
R₂′, R₃′, R₄′, R₅′, and R₆′ are independently H, F, Cl, Br, (CH₂)ₙCH₃, (CH₂ₙ)OH, O—(CH₂)ₙCH₃, O(CH₂)OH, (CH₂ₙ)SH, S—(CH₂)ₙCH₃, S(CH₂)ₙSH, O(CH₂)ₙSH, S(CH₂)ₙOH, (CH₂)ₙNR₈R₉, O(CH₂)ₙNR₈R₉, or S(CH₂)ₙNR₈R₉ or R₃′═OP(═O)(O-benzyl)₂;
wherein R₈ and R₉ are independently H, (CH₂)ₙCH₃, (CH₂ₙ)OH, (CH₂ₙ)SH, (CH₂)ₙN(CₙH₂ₙ₊₁)(CₘH₂ₘ₊₁); and
wherein n and m are each an integer selected from 0 to 4.

In one embodiment of the linker-payload of the present disclosure, one of R₂′, R₃′, R₄′, R₅′, and R₆′ is F or OCH₃. In one embodiment of the linker-payload of the present disclosure, one of R₂, R₃ and R₄ is O(CH₂)ₙCH₃ or (CH₂)ₙNR₈R₉, and the others thereof are H.

In one embodiment of the linker-payload of the present disclosure, the drug has a structure of the following Formula (I-1):

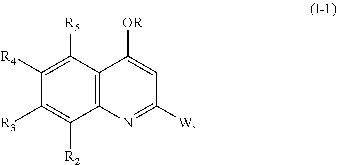

wherein the linking group (L) is connected to Formula (I-1) at R₅; the definitions of W, R, R₂, R³, R₄ and R₅ are the same as those defined above.

In one embodiment of the linker-payload of the present disclosure, the drug has a structure of the following Formula (I-2):

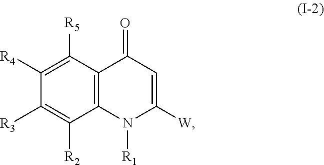

wherein the linking group (L) is connected to Formula (I-2) at R₅; the definitions of W, R₁, R₂, R³, R₄ and R₅ are the same as those defined above.

In one embodiment of the linker-payload of the present disclosure, the linking group (L) has a structure selected from the group consisting of:

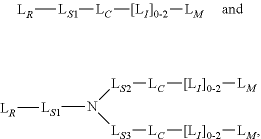

wherein, L_{R} is a reactive group that is capable of reacting with an antibody;
each of L_{S1}, L_{S2}, and L_{S3} is a spacer;
N is nitrogen;
L_{C} is a cleavable linker or a non-cleavable linker;
L_{I} is a self-immolative linker;
L_{M} is a moiety connecting to the R₅ of the drug (D).

In one embodiment of the linker-payload of the present disclosure, L_{R} comprises a thiol, maleimide, haloacetamide, vinyl sulfone, aziridine, azido, alkyne, cyclononyne, cyclooctyne, cyclooctene, triarylphosphine, oxanorbornadiene, diaryltetrazine, aryltetrazine, norbornene, aldehydes, hydroxylamine, hydrazine, NH₂—NH—C(═O)—, ketone, CoA or serine residue. In one embodiment of the linker-payload of the present disclosure, the L_{R} comprises bicyclononyne (BCN), dibenzocyclooctyne (DBCO), dibenzoannulated cyclooctyne (DIBO), sulfonylated DIBO (s-DIBO) or maleimidocaproyl (MC).

In one embodiment of the linker-payload of the present disclosure, L_{I} is selected from the group consisting of p-aminobenzylcarbamoyl (PAB) and dimethylethylenediamine (DMEA).

In one embodiment of the linker-payload of the present disclosure, each of the spacer is a PEG chain having 1 to 10 PEG units.

In one embodiment of the linker-payload of the present disclosure, L_{C} is a linker comprising a peptide bond, a phosphate bond, a nucleic acid bond, a sugar bond, a disulfide bond, an amide bond, a substituted amide bond in the form of a peptide bond, a thioamide bond, an ester bond, a thioester bond, a vicinal diol bond, or a hemiacetal. In one embodiment of the linker-payload of the present disclosure, L_{C} is a cleavable linker selected from the group consisting of cathepsin B, hydrazone, valine-citrulline (vc), phenylalanine-lysine, glycine-glycine-phenylalanine-glycine (GGFG), N-succinimidyl-3-(2-pyridyldithio) propionate (SPDP), N-hydroxysuccinimidyl 4-(2-pyridydithio) butanoate (SPDB), N-succinimidyl 4-(2-pyridyldithio) pentanoate (SPP), valine-alanine (va), mb-vc, CL2A, CL2E, cleavable vc-based linker, and fleximer polymer linker. In one embodiment of the linker-payload of the present disclosure, L_{C} is a non-cleavable linker selected from the group consisting of succinimidyl-4-(N-maleimidomethyl)cyclohexane-1-carboxylate (SMCC), maleimidocaproic acid (mc) and N-hydroxysuccinimidyl 4-(2-pyridyldithio)-2-sulfobutanoate (sulfo-SPDB).

In one embodiment of the linker-payload of the present disclosure, L_{M} is selected from the group consisting of —C(═O)—, C₁-C₁₈ alkylene group, —CH₂—CH═CH—, and phenylene group.

The present disclosure further provides an antibody-drug conjugate having a structure of Ab-[L-(D)ₚ]_{q},
wherein Ab is an antibody;
L is a linking group;
p is an integer selected from 1 to 3;
q is an integer selected from 1 to 8;
D is a drug having a structure of following Formula (I):

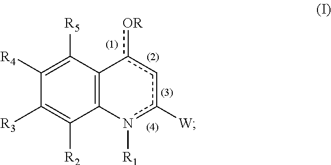

wherein the linking group (L) is connected to the drug (D) at R₅; the linking group (L) is connected between antibody (Ab) and drug (D);
R is absent or present, and wherein R, if present, is hydrogen, P(═O)(OH)₂, P(═O)(O(C₁-C₁₈)alkylene(C₆-C₂₀)aryl)₂, P(═O)(OH)(OM), P(═O)(OM)₂, P═O(O₂M), S(═O)(OH)₂, S(═O)(O(C₁-C₁₈)alkylene(C₆-C₂₀)aryl)₂, S(═O)(OH)(OM), S(═O)(OM)₂, wherein Mis a monovalent or divalent metal ion, or alkylammonium ion;
R₁ is absent or present, and wherein R₁, if present, is hydrogen;
R₂, R₃ and R₄ independently are H, F, Cl, Br, (CH₂)ₙCH₃, (CH₂)ₙOH, O(CH₂)ₙCH₃, O(CH₂)ₙOH, O(CH₂)ₙNR₈R₉, (CH₂)ₙSH, S(CH₂)ₙCH₃, S(CH₂)ₙSH, S(CH₂)ₙNR₈R₉, (CH₂)ₙNR₈R₉, (CH₂)ₙN, or R₃ and R₄ together is —O(CH₂)ₙO— or —S(CH₂)ₙS—;
R₅ is O;
each of bonds (1) to (4) independently represents a single bond or a double bond, provided that when bond (1) is a single bond, R is present, R₁ is absent, each of bonds (2) and (4) is a double bond, and bond (3) is a single bond; and when bond (1) is a double bond, R is absent, R₁ is present, each of bonds (2) and (4) is a single bond, and bond (3) is a double bond;
W is an aromatic group selected from the group consisting of naphthyl, quinolinyl, benzofuranyl, benzothiophenyl, anthracenyl, and substituted phenyl of formula (Y):

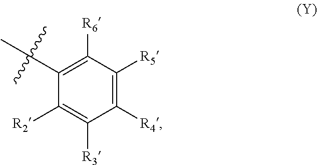

and wherein:
R₂′, R₃′, R₄′, R₅′, and R₆′ are independently H, F, Cl, Br, (CH₂)ₙCH₃, (CH₂ₙ)OH, O—(CH₂)ₙCH₃, O(CH₂)OH, (CH₂ₙ)SH, S—(CH₂)ₙCH₃, S(CH₂)ₙSH, O(CH₂)SH, S(CH₂)ₙOH, (CH₂)ₙNR₈R₉, O(CH₂)ₙNR₈R₉, or S(CH₂)ₙNR₈R₉ or R₃′═OP(═O)(O-benzyl)₂;
wherein R₈ and R₉ are independently H, (CH₂)ₙCH₃, (CH₂ₙ)OH, (CH₂ₙ)SH, (CH₂)ₙN(CₙH₂ₙ₊₁)(CₘH₂ₘ₊₁); and
wherein n and m are each an integer selected from 0 to 4.

In one embodiment of the antibody-drug conjugate of the present disclosure, one of R₂′, R₃′, R₄′, R₅′, and R₆′ is F or OCH₃. In one embodiment of the composition of the present disclosure, one of R₂, R₃ and R₄ is O(CH₂)ₙCH₃ or (CH₂)ₙNR₈R₉, and the others thereof are H.

In one embodiment of the antibody-drug conjugate of the present disclosure, the drug has a structure of the following Formula (I-1):

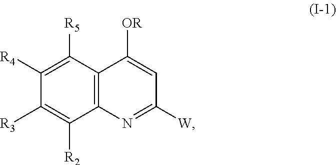

wherein the linking group (L) is connected to Formula (I-1) at R₅; the linking group (L) is connected between antibody (Ab) and Formula (I-1); the definitions of W, R, R₂, R³, R₄ and R₅ are the same as those defined in the above.

In one embodiment of the antibody-drug conjugate of the present disclosure, the drug has a structure of the following Formula (I-2):

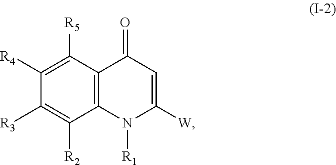

wherein the linking group (L) is connected to the Formula (I-2) at R₅; the linking group (L) is connected between antibody (Ab) and Formula (I-2); the definitions of W, R₁, R₂, R³, R₄ and R₅ are the same as those defined in the above.

In one embodiment of the antibody-drug conjugate of the present disclosure, the Drug-to-Antibody Ratio (DAR) of the antibody-drug conjugate is between 1 to 24.

In one embodiment of the antibody-drug conjugate of the present disclosure, the linking group (L) has a structure selected from the group consisting of:

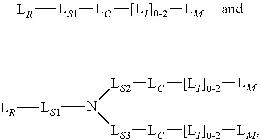

wherein the definitions of L_{R}, L_{S1}, L_{S2}, L_{S3}, L_{C}, L_{I} and L_{M} are the same as those defined in the above.

In one embodiment of the antibody-drug conjugate of the present disclosure, L_{R} comprises a thiol, maleimide, haloacetamide, vinyl sulfone, aziridine, azido, alkyne, cyclononyne, cyclooctyne, cyclooctene, triarylphosphine, oxanorbornadiene, diaryltetrazine, aryltetrazine, norbornene, aldehydes, hydroxylamine, hydrazine, NH₂—NH—C(═O)—, ketone, CoA or serine residue. In one embodiment of the antibody-drug conjugate of the present disclosure, the L_{R} comprises bicyclononyne (BCN), dibenzocyclooctyne (DBCO), dibenzoannulated cyclooctyne (DIBO), sulfonylated DIBO (s-DIBO) or maleimidocaproyl (MC).

In one embodiment of the antibody-drug conjugate of the present disclosure, L_{I} is selected from the group consisting of p-aminobenzylcarbamoyl (PAB) and dimethylethylenediamine (DMEA).

In one embodiment of the antibody-drug conjugate of the present disclosure, each of the spacer is a PEG chain having 1 to 10 PEG units.

In one embodiment of the antibody-drug conjugate of the present disclosure, L_{C} is a linker comprising a peptide bond, a phosphate bond, a nucleic acid bond, a sugar bond, a disulfide bond, an amide bond, a substituted amide bond in the form of a peptide bond, a thioamide bond, an ester bond, a thioester bond, a vicinal diol bond, or a hemiacetal. In one embodiment of the antibody-drug conjugate of the present disclosure, L_{C} is a cleavable linker selected from the group consisting of cathepsin B, hydrazone, valine-citrulline (vc), phenylalanine-lysine, glycine-glycine-phenylalanine-glycine (GGFG), N-succinimidyl-3-(2-pyridyldithio) propionate (SPDP), N-hydroxysuccinimidyl 4-(2-pyridydithio) butanoate (SPDB), N-succinimidyl 4-(2-pyridyldithio) pentanoate (SPP), valine-alanine (va), mb-vc, CL2A, CL2E, cleavable vc-based linker, and fleximer polymer linker. In one embodiment of the antibody-drug conjugate of the present disclosure, L_{C} is a non-cleavable linker selected from the group consisting of succinimidyl-4-(N-maleimidomethyl)cyclohexane-1-carboxylate (SMCC), maleimidocaproic acid (mc) and N-hydroxysuccinimidyl 4-(2-pyridyldithio)-2-sulfobutanoate (sulfo-SPDB).

In one embodiment of the antibody-drug conjugate of the present disclosure, L_{M} is selected from the group consisting of —C(═O)—, C₁-C₁₈ alkylene group, —CH₂—CH═CH—, and phenylene group.

In one embodiment of the antibody-drug conjugate of the present disclosure, the antibody comprises a glycan, a modified glycan or a functional group that is capable of undergoing a reaction with L_{R}.

In one embodiment of the antibody-drug conjugate of the present disclosure, the modified glycan comprises a thiol, maleimide, haloacetamide, vinyl sulfone, aziridine, azido, alkyne, cyclononyne, cyclooctyne, cyclooctene, triarylphosphine, oxanorbornadiene, diaryltetrazine, aryltetrazine, norbornene, aldehydes, hydroxylamine, hydrazine, NH₂—NH—C(═O)—, ketone, CoA or serine residue.

In one embodiment of the antibody-drug conjugate of the present disclosure, the antibody is selected form the group consisting of an anti-HER2 antibody, an anti-EGFR antibody, an anti-PD-L1 antibody, an anti-VEGF antibody, an anti-HER3 antibody, an anti-TROP2 antibody, an anti-MET antibody, an anti-ROR1 antibody, an anti-ROR2 antibody, anti-BCMA antibody, anti-Mesothelin antibody, an anti-B7-H3 antibody, an anti-B7-H4 antibody, an anti-GPR20 antibody, an anti-tissue factor (TF) antibody, an anti-folate receptor α (FRα) antibody, an anti-Nectin-4 antibody, an anti-Somatostatin receptor 2 (SSTR2) antibody, anti-SSTR5 antibody, an anti-Claudin 18.2 antibody, an anti-LIV-1 antibody, an anti-Prostate-specific membrane antigen (PSMA) antibody, an anti-AXL antibody, an anti-CEACAM5 antibody, an anti-IGFIR antibody, an anti-EPHA2 antibody, an anti-MUC1 antibody, an anti-KIT antibody, an anti-DLL3 antibody, an anti-NaPi-2b antibody, an anti-MSLN antibody, an anti-5T4 antibody, an anti-CDH6 antibody, an anti-CDH7 antibody, an anti-CD37 antibody, an anti-CD30 antibody, and an anti-CD20 antibody, an anti-CD19 antibody, an anti-CD22 antibody, an anti-CD33 antibody, an anti-CD45 antibody, an anti-CD70 antibody, an anti-CD79B antibody and an anti-CD142 antibody.

The present disclosure further provides a method for treating a cancer, comprising administering to a subject in need thereof a therapeutically effective amount of a linker-payload or an antibody-drug conjugate of the present disclosure.

The present disclosure further provides a use of a linker-payload or an antibody-drug as defined above in the manufacture of a medicament for treating a cancer.

In some embodiments of the method or the use of the present disclosure, the cancer is selected from breast cancer, gastric cancer, ovarian cancer, lung cancer, colon cancer, liver cancer, brain cancer, prostate cancer, melanoma, pancreatic cancer, head and neck cancer, neuroendocrine neoplasia, but are not limited thereto.

The present disclosure further provides a linker-payload or an antibody-drug conjugate of the present disclosure for use as a medicament. In some embodiments of the linker-payload or antibody-drug conjugate, the linker-payload or antibody-drug conjugate of the present disclosure is used in the treatment of a cancer.

In some embodiments of the linker-payload or antibody-drug conjugate, the cancer is selected from breast cancer, gastric cancer, ovarian cancer, lung cancer, colon cancer, liver cancer, brain cancer, prostate cancer, melanoma, pancreatic cancer, head and neck cancer, neuroendocrine neoplasia, but are not limited thereto.

The present disclosure further provides a compound having a structure of the following Formula (II):

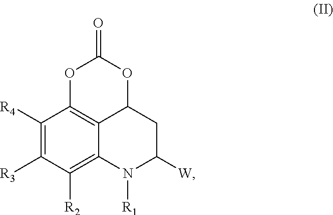

wherein R₁ is hydrogen;
R₂, R₃ and R₄ independently are H, F, Cl, Br, (CH₂)ₙCH₃, (CH₂)ₙOH, O(CH₂)ₙCH₃, O(CH₂)ₙOH, O(CH₂)ₙNR₈R₉, (CH₂)ₙSH, S(CH₂)ₙCH₃, S(CH₂)ₙSH, S(CH₂)ₙNR₈R₉, (CH₂)ₙNR₈R₉, (CH₂)ₙN, or R₃ and R₄ together is —O(CH₂)ₙO— or —S(CH₂)ₙS—;
W is an aromatic group selected from the group consisting of naphthyl, quinolinyl, benzofuranyl, benzothiophenyl, anthracenyl, and substituted phenyl of formula (Y):

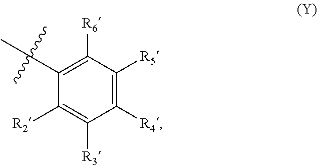

and wherein:
R₂′, R₃′, R₄′, R₅′, and R₆′ are independently H, F, Cl, Br, (CH₂)ₙCH₃, (CH₂ₙ)OH, O—(CH₂)ₙCH₃, O(CH₂)OH, (CH₂ₙ)SH, S—(CH₂)ₙCH₃, S(CH₂)ₙSH, O(CH₂)ₙSH, S(CH₂)ₙOH, (CH₂)ₙNR₈R₉, O(CH₂)ₙNR₈R₉, or S(CH₂)ₙNR₈R₉ or R₃′═OP(═O)(O-benzyl)₂;
wherein R₈ and Ry are independently H, (CH₂)ₙCH₃, (CH₂ₙ)OH, (CH₂ₙ)SH, (CH₂)ₙN(CₙH₂ₙ₊₁)(CₘH₂ₘ₊₁); and
wherein n and m are each an integer selected from 0 to 4.

In some embodiments of the compound, the compound is made by reacting a drug having a structure of following Formula (I) with 4-nitrobenzyl chloroformate:

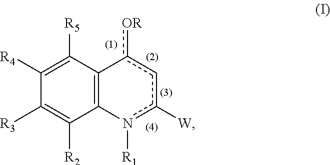

wherein the definitions of W, R, R₁, R₂, R³, R₄ and R⁵ are the same as those defined in the above.

The linker-payload thus obtained can be further conjugate with an antibody so as to provide an antibody-drug conjugate. No observable aggregates were detected in the antibody-drug conjugate, and the antibody-drug conjugate of the present disclosure is well tolerated. The antibody-drug conjugate of the present disclosure also exhibited significant antitumor efficacy in vivo pharmacology study results. These studies demonstrated the linker-payload and antibody-drug conjugate of the present disclosure are useful for treating cancer.

### BRIEF DESCRIPTION OF THE DRAWINGS

The present disclosure can be more fully understood by reading the following descriptions of the embodiments, with reference made to the accompanying drawings.

FIGS. 1A-1B are graphs showing the mean percent body weight changes (A) and average tumor growth curves (B) over time in BT-474 xenograft model treated with vehicle, CVM-ADC01, CVM-ADC20 and CVM-ADC23 (5 mg/kg) on Day 27.

FIGS. 2A-2C are graphs showing the mean percent body weight changes (A), average tumor growth curves (B) and tumor weights (C) over time in BT-474 xenograft model treated with vehicle, CVM-ADC01 (5 mg/kg or 10 mg/kg) or reference drug (Herceptin) on Day 28.

FIGS. 3A-3C are graphs showing the body weight changes (A), average tumor growth curves (B) and tumor weight (C) in BT-474 xenograft model treated with vehicle, CVM-ADC01, CVM-ADC02 (5 mg/kg) or reference drug (Enhertu) on Day 28.

FIGS. 4A-4B are graphs showing the body weight changes (A) and average tumor volume curves (B) in MDA-MB-468 xenograft model treated with vehicle, CVM-ADC10 (5 mg/kg) or reference drug (Cetuximab) on Day 27.

### DETAILED DESCRIPTION OF THE PREFERRED EMBODIMENTS

The following examples are used for illustrating the present disclosure. A person skilled in the art can easily conceive the other advantages and effects of the present disclosure, based on the disclosure of the specification. The present disclosure can also be implemented or applied as described in different examples. It is possible to modify or alter the following examples for carrying out this disclosure without contravening its scope, for different aspects and applications.

Abbreviations as used in the present disclosure are listed below for reference:

TABLE 1

| | |
|---|---|
| ABC | Ammonium Bicarbonate |
| ADC | Antibody drug conjugate |
| DAR | Drug-to-Antibody Ratio |
| DIPEA | N,N-Diisopropylethylamine |
| DMEA | 1,2-Dimethylethylenediamine |
| DMAP | 4-Dimethylaminopyridine |
| DMF | Dimethylformamide |
| DMSO | Dimethyl sulfoxide |
| DTT | Dithiothreitol |
| EDC | 1-Ethyl-3-(3-dimethylaminopropyl)carbodiimide |
| EDTA | Ethylenediaminetetraacetic acid |
| ELISA | Enzyme-linked immunosorbent assay |
| eq | equivalent |
| EtOAc | Ethyl acetate |
| HATU | Hexafluorophosphate azabenzotriazole |
| | tetramethyl uronium |
| HEPES | (4-(2-hydroxyethyl)-1-piperazineethanesulfonic acid |
| Her2/ERBB2 | human epidermal growth factor receptor 2 |
| HIC | Hydrophobic Interaction Chromatography |
| HPLC | High Performance Liquid Chromatography |
| hr/h | hour |
| HRMS | High-resolution mass spectrometry |
| LC-MS | Liquid Chromatography Mass Spectrometry |
| mg | milligram |
| min | minute |
| mL | milliliter |
| μL | microliter |
| mm | millimeter |
| mM | millimolar |
| NAC | Acetylcysteine |
| NHS | N-Hydroxysuccinimide |
| nm | nanometer |
| NMR | Nuclear Magnetic Resonance |
| NMWL | nominal molecular weight limit |
| PBS | Phosphate buffered saline |
| PBS | Phosphate buffered saline |
| RP | Reverse phase |
| rpm | Revolution(s) Per Minute |
| SEC | Size Exclusion Chromatography |
| TCEP | Tris(2-carboxyethyl)phosphine hydrochloride |
| TFA | Trifluoroacetic acid |
| UPLC | Ultra Performance Liquid Chromatographic |

It should be noted that as used herein and in the appended claims, the singular forms “a,” “an,” and “the” include plural reference unless the context clearly dictates otherwise. As well, the terms “a” (or “an”), “one or more” and “at least one” can be used interchangeably herein. It is also to be noted that the terms “comprising,” “including,” and “having” can be used interchangeably.

When referring to a drug component of the linker-payload or antibody-drug conjugate of the present disclosure, it is intended that the drug encompass not only the specified molecular entity but also its pharmaceutically acceptable analogs, including, but not limited to, salts, esters, amides, active metabolites, and other such derivatives, analogs, and related compounds.

The present disclosure is directed to a linker-payload having a structure of L-(D)ₚ,
wherein L is a linking group;
p is an integer selected from 1 to 3,
D is a drug having a structure of the following Formula (I):

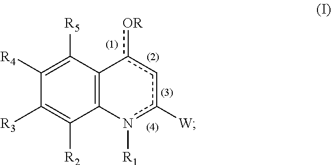

wherein the linking group (L) is connected to the drug (D) at R₅;
R is absent or present, and wherein R, if present, is hydrogen, P(═O)(OH)₂, P(═O)(O(C₁-C₁₈)alkylene(C₆-C₂₀)aryl)₂, P(═O)(OH)(OM), P(═O)(OM)₂, P═O(O₂M), S(═O)(OH)₂, S(═O)(O(C₁-C₁₈)alkylene(C₆-C₂₀)aryl)₂, S(═O)(OH)(OM), S(═O)(OM)₂, wherein Mis a monovalent or divalent metal ion, or alkylammonium ion;
R₁ is absent or present, and wherein R₁, if present, is hydrogen;
R₂, R₃ and R₄ independently are H, F, Cl, Br, (CH₂)ₙCH₃, (CH₂)ₙOH, O(CH₂)ₙCH₃, O(CH₂)ₙOH, O(CH₂)ₙNR₈R₉, (CH₂)ₙSH, S(CH₂)ₙCH₃, S(CH₂)ₙSH, S(CH₂)ₙNR₈R₉, (CH₂)ₙNR₈R₉, (CH₂)ₙN, or R₃ and R₄ together is —O(CH₂)ₙO— or —S(CH₂)ₙS—;
R₅ is O;
each of bonds (1) to (4) independently represents a single bond or a double bond, provided that when bond (1) is a single bond, R is present, R₁ is absent, each of bonds (2) and (4) is a double bond, and bond (3) is a single bond; and when bond (1) is a double bond, R is absent, R₁ is present, each of bonds (2) and (4) is a single bond, and bond (3) is a double bond;
W is an aromatic group selected from the group consisting of naphthyl, quinolinyl, benzofuranyl, benzothiophenyl, anthracenyl, and substituted phenyl of formula (Y):

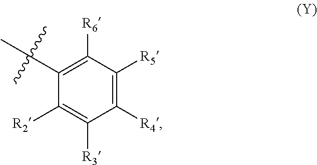

and wherein:
R₂′, R₃′, R₄′, R₅′, and R₆′ are independently H, F, Cl, Br, (CH₂)ₙCH₃, (CH₂ₙ)OH, 0-(CH₂)ₙCH₃, O(CH₂)OH, (CH₂ₙ)SH, S—(CH₂)ₙCH₃, S(CH₂)ₙSH, O(CH₂)ₙSH, S(CH₂)ₙOH, (CH₂)ₙNR₈R₉, O(CH₂)ₙNR₈R₉, or S(CH₂)ₙNR₈R₉ or R₃′═OP(═O)(O-benzyl)₂;
wherein R₅ and R₉ are independently H, (CH₂)ₙCH₃, (CH₂ₙ)OH, (CH₂ₙ)SH, (CH₂)ₙN(CₙH₂ₙ₊₁)(CₘH₂ₘ₊₁); and
wherein n and m are each an integer selected from 0 to 4.

In some embodiments, the present application relates to two forms of drugs having the following structures of Formula (I-1) and Formula (I-2), respectively. A drug (D) having the structure of Formula (I-1) can convert into a drug (D) having the structure of Formula (I-2), and vice versa.

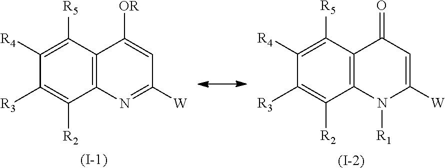

The definitions of W, R, R₁, R₂, R³, R₄ and R₅ are the same as those defined in the above.

In some embodiments, in the drug (D) having the structure of Formula (I) of the present disclosure, one of R₂′, R₃′, R₄′, R₅′, and R₆′ is F or OCH₃ and/or one of R₂, R₃ and R₄ is O(CH₂)ₙCH₃ or (CH₂)ₙNR₈R₉, and the others thereof are H. In some embodiments, the drug (D) having the structure of Formula (I) of the present disclosure can be made into a linker-payload by connecting a linking group (L) to R₅ of the drugs provided in the U.S. Pat. No. 8,524,740 B2 and U.S. Pat. No. 9,717,721 B2, which are hereby incorporated by reference herein and made a part of this specification.

In some embodiments, the drug (D) having the structure of Formula (I) of the present disclosure is selected from Table 2, but not limited thereto.

TABLE 2

| No. | Structure | Substituents |
|---|---|---|
| 1. | 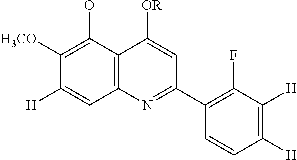 | R, R₅′, R₄′, R₃′, R₂′ = H R₆′ = fluoro R₄ = methoxy R₅ = O R₃, R₂ = H |
| 2. | 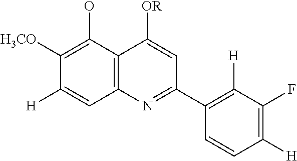 | R, R₆′, R₄′, R₃′, R₂′ = H R₅′ = fluoro R₄ = methoxy R₅ = O R₃, R₂ = H |
| 3. | 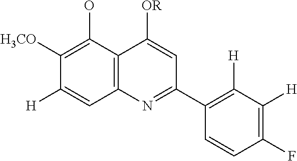 | R, R₆′, R₅′, R₃′, R₂′ = H R₄′ = fluoro R₄ = methoxy R₅ = O R₃, R₂ = H |
| 4. | 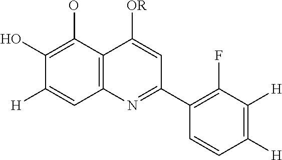 | R, R₅′, R₄′, R₃′, R₂′ = H R₆′= fluorol R₄ = hydroxyl R₅ = O R₃, R₂ = H |
| 5. | 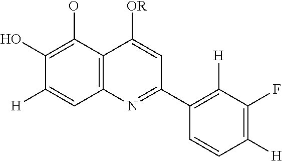 | R, R₆′, R₄′, R₃′, R₂′ = H R₅′ = fluoro R₄ = hydroxyl R₅ = O R₃, R₂ = H |
| 6. | 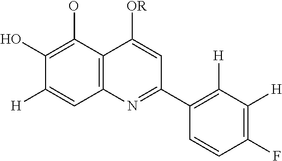 | R, R₆′, R₅′, R₃′, R₂′ = H R₄′ = fluoro R₄ = hydroxyl R₅ = O R₃, R₂ = H |
| 7. | 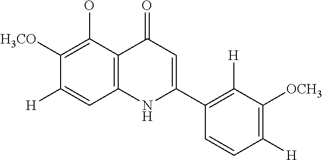 [SMILES: COc1cccc(-c2cc(=O)c3c(O)c(OC)ccc3[nH]2)c1] | R₆′, R₄′, R₃′, R₂′ = H R₅′ = OCH₃ R₄ = OCH₃ R₅ = O R₁, R₃, R₂ = H |
| 8. | 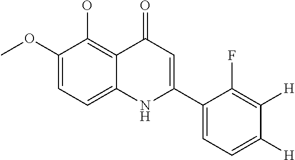 [SMILES: COc1ccc2[nH]c(-c3ccccc3F)cc(=O)c2c1O] | R₅′, R₄′, R₃′, R₂′ = H R₆′ = fluoro R₄ = OCH₃ R₅ = O R₁, R₃, R₂ = H |
| 9. | 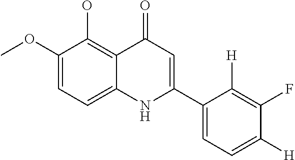 [SMILES: COc1ccc2[nH]c(-c3cccc(F)c3)cc(=O)c2c1O] | R₆′, R₄′, R₃′, R₂′ = H R₅′ = fluoro R₄ = OCH₃ R₅ = O R₁, R₃, R₂ = H |
| 10. | 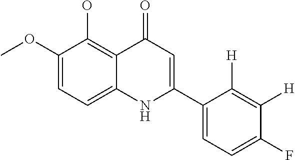 [SMILES: COc1ccc2[nH]c(-c3ccc(F)cc3)cc(=O)c2c1O] | R₆′, R₅′, R₃′, R₂′ = H R₄′ = fluoro R₄ = OCH₃ R₅ = O R₁, R₃, R₂ = H |
| 11. | 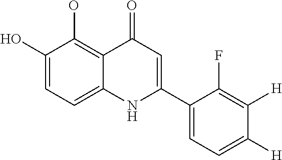 [SMILES: O=c1cc(-c2ccccc2F)[nH]c2ccc(O)c(O)c12] | R₄′, R₅′, R₃′, R₂′ = H R₆′ = fluoro R₄ = hydroxyl R₅ = O R₁, R₃, R₂ = H |
| 12. | 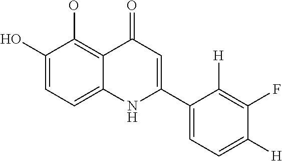 [SMILES: O=c1cc(-c2cccc(F)c2)[nH]c2ccc(O)c(O)c12] | R₆′, R₄′, R₃′, R₂′ = H R₅′ = fluoro R₄ = hydroxyl R₅ = O R₁, R₃, R₂ = H |
| 13. | 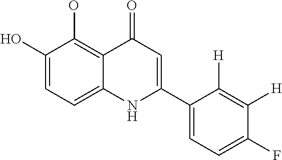 [SMILES: O=c1cc(-c2ccc(F)cc2)[nH]c2ccc(O)c(O)c12] | R₆′, R₅′, R₃′, R₂′ = H R₄′ = fluoro R₄ = hydroxyl R₅ = O R₁, R₃, R₂ = H |

In another embodiment, the drug (D) having the structure of Formula (I) of the present disclosure is the following compound:

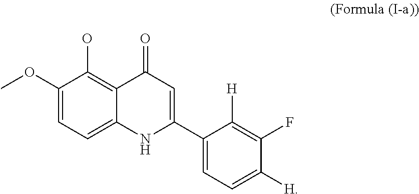

In some embodiments, the linking group (L) in the linker-payload of the present disclosure has a structure selected from the group consisting of:

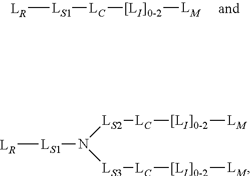

wherein, L_{R} is a reactive group that is capable of reacting with an antibody; each of L_{S1}, L_{S2}, and L_{S3} is a spacer; N is nitrogen; L_{C} is a cleavable linker or a non-cleavable linker; L_{I} is a self-immolative linker; L_{M} is a moiety connecting to the R₅ of the drug (D).

The term “L_{R}” used in the present disclosure refers to a component of the linking group (L), wherein this component has a functional group that can react with another functional group on an antibody. The examples of functional groups being comprised in the L_{R}, are thiol, maleimide, haloacetamide, vinyl sulfone, aziridine, azido, alkyne, cyclononyne, cyclooctyne, cyclooctene, triarylphosphine, oxanorbornadiene, diaryltetrazine, aryltetrazine, norbornene, aldehydes, hydroxylamine, hydrazine, NH₂—NH—C(═O)—, ketone, CoA or serine residue.

In some embodiments, the functional groups being comprised in the L_{R} and the functional groups being comprised in the antibody are different. In some embodiments, the functional groups being comprised in the L_{R} and the functional groups being comprised in the antibody are the same. For examples, if L_{R} comprises a thiol, the antibody can comprise a thiol, maleimide or haloacetamide; and if L_{R} comprises an azido, the antibody can comprise an alkyne, triarylphosphine, cyclooctyne or oxanorbornadiene. In a preferably embodiment, L_{R} comprises bicyclononyne (BCN), dibenzocyclooctyne (DBCO), dibenzoannulated cyclooctyne (DIBO) or sulfonylated DIBO (s-DIBO) and the antibody comprises an azido. In another embodiment, L_{R} comprises maleimidocaproyl (MC), and the antibody comprises a cysteine thiol (—SH) group.

Optionally, L_{R} further comprises a reacting group at the terminal to attach to the remainder of the linking group. For example, when L_{R} comprises dibenzocyclooctyne (DBCO), it may further comprise a carboxyl group, an amine group, a NHS ester or a maleimide attached to either the cyclooctyne ring or the nitrogen atom within the cyclooctyne ring.

The term “L_{I}” used in the present disclosure refers to a component of the linking group, wherein this component will self-immolate in an organism and thus release a chemically unmodified drug (i.e., without an adduct, such as a portion of the cleavable linker, on the drug). In some embodiments, L_{I} can be present or absent, and wherein L_{I}, if present, can be selected from the group consisting of p-aminobenzylcarbamoyl (PAB) and dimethylethylenediamine (DMEA); alternatively, the linking group (L) of the present disclosure may also comprise more than one L_{I}, such as two L_{I} that are linked continuously, for example, PAB-DMEA.

The term “L_{S1},” “L_{S2}” and “L_{S3}” refers to spacers that comprised in the linking group. L_{S2} and L_{S3} can be absent or present, and wherein L_{S2} and L_{S3}, if present, are connected to L_{S1} by a nitrogen atom.

In some embodiments, each of the spacer is a PEG chain having 1 to 10 PEG units, for example 1, 2, 3, 4, 5, 6, 7, 8, 9, or 10 PEG units. The term “PEG” used herein represents polyethylene glycol, which is an organic moiety comprising repeating ethylene-oxy subunits covalently attached to each other. The subunits are linked together, for example, in a linear, branched or star shaped configuration. Optionally, the terminal PEG unit in each of spacer may be modified to attach to the remainder of the linking group. The spacers can be the same or different from each other. For example, when L_{S1} is a PEG chain having 1 to 6 PEG units, L_{S2} and L_{S3} can independently be absent or a PEG chain having 1 to 5 PEG units; preferably, when L_{S1} is a PEG chain having 5 PEG units, L_{S2} and L_{S3} are both a PEG chain having 4 PEG units.

The term “L_{C}” used in the present disclosure refers to a cleavable linker or a non-cleavable linker. The cleavable or non-cleavable linkers that can be used in the linker-payload or anti-body conjugate are well known in the art, and linkers comprising a peptide bond, a phosphate bond, a nucleic acid bond, a sugar bond, a disulfide bond, an amide bond, a substituted amide bond in the form of a peptide bond, a thioamide bond, an ester bond, a thioester bond, a vicinal diol bond, or a hemiacetal are all encompassed in the present disclosure.

In some embodiments, when the L_{C} is a cleavable linker, it can be cleaved by several ways (e.g., by enzymes) in the target place, leading to efficient release of the active agent (i.e., drug). In some embodiments, the cleavable linker is selected from the group consisting of cathepsin B, hydrazone, valine-citrulline (vc), phenylalanine-lysine, glycine-glycine-phenylalanine-glycine (GGFG), N-succinimidyl-3-(2-pyridyldithio) propionate (SPDP), N-hydroxysuccinimidyl 4-(2-pyridydithio) butanoate (SPDB), N-succinimidyl 4-(2-pyridyldithio) pentanoate (SPP), valine-alanine (va), mb-vc, CL2A, CL2E, cleavable vc-based linker, and fleximer polymer linker.

In some embodiments, when the L_{C} is a non-cleavable linker, the non-cleavable linker is selected from the group consisting of succinimidyl-4-(N-maleimidomethyl)cyclohexane-1-carboxylate (SMCC), maleimidocaproic acid (mc) and N-hydroxysuccinimidyl 4-(2-pyridyldithio)-2-sulfobutanoate (sulfo-SPDB).

The term “L_{M}” used in the present disclosure refers to a component of the linking group (L), which connects to the R₅ of the drug (D). In some embodiments, L_{M} is selected from the group consisting of —C(═O)—, C₁-C₁₈ alkylene group, —CH₂—CH═CH—, and phenylene group. In some embodiments, L_{M} is —C(═O)—.

In some embodiments, the type of L_{M} depends on the activation step of the synthesis process of the linker-payload of the present disclosure and the selections of L_{C} and/or L_{I}. In some embodiments, by reacting the drug (D) of Formula (I) as defined above with suitable active agent, an active form of drug (i.e., an activation intermediate) having a structure of following Formula (II) can thus be provided, and then the compound of Formula (II) can react with L_{C} and/or L_{I}, so as to form L_{M}:

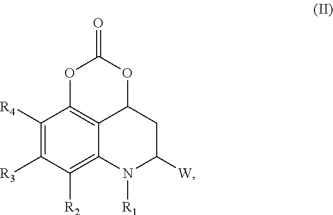

wherein R₁ is hydrogen;
R₂, R₃ and R₄ independently are H, F, Cl, Br, (CH₂)ₙCH₃, (CH₂)ₙOH, O(CH₂)ₙCH₃, O(CH₂)OH, O(CH₂)ₙNR₈R₉, (CH₂)ₙSH, S(CH₂)ₙCH₃, S(CH₂)ₙSH, S(CH₂)ₙNR₈R₉, (CH₂)ₙNR₈R₉, (CH₂)ₙN, or R₃ and R₄ together is —O(CH₂)ₙO— or —S(CH₂)ₙS—;
W is an aromatic group selected from the group consisting of naphthyl, quinolinyl, benzofuranyl, benzothiophenyl, anthracenyl, and substituted phenyl of formula (Y):

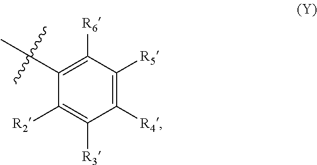

and wherein:
R₂′, R₃′, R₄′, R₅′, and R₆′ are independently H, F, Cl, Br, (CH₂)ₙCH₃, (CH₂ₙ)OH, O—(CH₂)ₙCH₃, O(CH₂)OH, (CH₂ₙ)SH, S—(CH₂)ₙCH₃, S(CH₂)ₙSH, O(CH₂)ₙSH, S(CH₂)ₙOH, (CH₂)ₙNR₈R₉, O(CH₂)ₙNR₈R₉, or S(CH₂)ₙNR₈R₉ or R₃′═OP(═O)(O-benzyl)₂;
wherein R₅ and R₉ are independently H, (CH₂)ₙCH₃, (CH₂ₙ)OH, (CH₂ₙ)SH, (CH₂)ₙN(CₙH₂ₙ₊₁)(CₘH₂ₘ₊₁); and
wherein n and m are each an integer selected from 0 to 4.

For example, when a drug having a structure of following Formula (I-a), i.e., CVM-1125, is used to provide the linker-payload, 4-nitrobenzyl chloroformate can be used to activate the drug of Formula (I-a), and thus provides an active form of drug having a structure of following Formula (II-a):

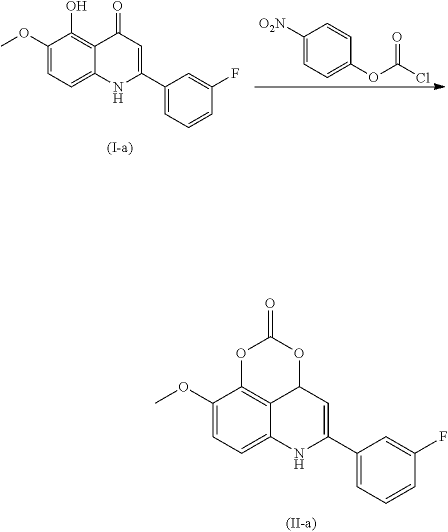 [SMILES: COc1ccc2[nH]c(-c3cccc(F)c3)cc(=O)c2c1O ; O=C(Cl)Oc1ccc([N+](=O)[O-])cc1 ; COc1ccc2c3c1OC(=O)OC3C=C(c1cccc(F)c1)N2]

After being activated, the compound of Formula (II-a) can be further react with a linking group as defined above, and thus form L_{M} as shown below:

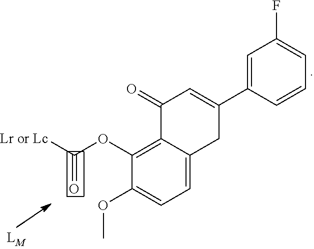

In another embodiment, the present disclosure provides an antibody-drug conjugate made by further conjugating an antibody to the linker-payload of the present disclosure as defined above.

In some embodiments, the antibody used for preparing the antibody-drug conjugate is selected according to the disease to be treated. A native intact antibody generally is a Y-shaped tetrameric protein comprising two heavy (H) and two light (L) polypeptide chains held together by covalent disulfide bonds and non-covalent interactions. Each heavy chain consists of a heavy chain variable region (VH) and a heavy chain constant region (CH), and each light chain consists of a light chain variable region (VL) and a light chain constant region (CL). VH and VL region can further be divided into hypervariable regions (called complementary determining regions (CDR)), which are interspaced by relatively conservative regions (called framework region (FR)). Each VH and VL consists of 3 CDRs and 4 FRs. The variable region (VH and VL) of each heavy/light chain pair forms antigen binding sites, respectively. The extent of the framework region and CDRs can be precisely identified using methodology known in the art.

In some embodiments, the antibodies suitable for the present disclosure comprises anti-HER2 antibody, an anti-EGFR antibody, an anti-PD-L1 antibody, an anti-VEGF antibody, an anti-HER3 antibody, an anti-TROP2 antibody, an anti-MET antibody, an anti-ROR1 antibody, an anti-ROR2 antibody, anti-BCMA antibody, anti-Mesothelin antibody, an anti-B7-H3 antibody, an anti-B7-H4 antibody, an anti-GPR20 antibody, an anti-tissue factor (TF) antibody, an anti-folate receptor α (FRα) antibody, an anti-Nectin-4 antibody, an anti-Somatostatin receptor 2 (SSTR2) antibody, anti-SSTR5 antibody, an anti-Claudin 18.2 antibody, an anti-LIV-1 antibody, an anti-Prostate-specific membrane antigen (PSMA) antibody, an anti-AXL antibody, an anti-CEACAM5 antibody, an anti-IGFIR antibody, an anti-EPHA2 antibody, an anti-MUC1 antibody, an anti-KIT antibody, an anti-DLL3 antibody, an anti-NaPi-2b antibody, an anti-MSLN antibody, an anti-5T4 antibody, an anti-CDH6 antibody, an anti-CDH7 antibody, an anti-CD37 antibody, an anti-CD30 antibody, and an anti-CD20 antibody, an anti-CD19 antibody, an anti-CD22 antibody, an anti-CD33 antibody, an anti-CD45 antibody, an anti-CD70 antibody, an anti-CD79B antibody, an anti-CD142 antibody but are not limited thereto.

In some embodiments, the antibody used for preparing the antibody-drug conjugate of the present disclosure is an anti-HER2 antibody. Preferably, the antibody comprises an amino acid sequence that is at least 60%, 70%, 75%, 80%, 85%, 90%, 95%, 96%, 97%, 98%, 99% or 100% identical to that following CDRs: CDRH1 consisting of an amino acid sequence GFNIKDTY, CDRH2 consisting of an amino acid sequence IYPTNGYT, CDRH3 consisting of an amino acid sequence SRWGGDGFYAMDY, CDRL1 consisting of an amino acid sequence QDVNTA, CDRL2 consisting of an amino acid sequence SAS, and CDRL3 consisting of an amino acid sequence QQHYTTPPT. In some embodiments, the antibody used for preparing the antibody-drug conjugate of the present disclosure is Trastuzumab (Herceptin).

In other embodiments, the antibody used for preparing the antibody-drug conjugate of the present disclosure can also be any antibody that can bind to the target molecular, such as Cetuximab, Panitumumab, Gefitinib, Atezolizumab, Avelumab, Durvalumab, Bevacizumab, Ranibizumab, Lumretuzumab, Sacituzumab, Obrindatamab, Naratuximab, Brentuximab, Rituximab and Ofatumumab.

The antibodies have one conserved oligosaccharide chain at the Asn297 residue in the CH₂ domain of each heavy chain. In some embodiments, the linking group is connected to the glycan or modified glycan at Asn297 via L_{R}. However, it should be understood that the linking group (L) can also bind to the antibody (Ab) through glycan or modified glycan at other positions; alternatively, the linking group may also connect to the antibody via other methods known in the art, such as targeting to the cysteine thiol (—SH) groups on the antibody.

In some embodiments, the antibody used in the present disclosure can connect to one or more linker-payloads, for example, the antibody can connect to 1 to 8 linker-payloads, i.e., the “q” in the structure “Ab-[L-(D)ₚ]_{q}” is an integer selected from 1, 2, 3, 4, 5, 6, 7 or 8. In some embodiments, each linker can connect to more than one drug, for example, each linker can connect to 1, 2 or 3 drugs, i.e., the “p” is an integer selected from 1, 2, or 3. Therefore, the Drug-to-Antibody Ratio (DAR) of the present disclosure is between 1 to 24. In some embodiments, the DAR of the present disclosure is about 4 or 8. As the DAR in the present disclosure is presented in an average value, the DAR may not be an integer, for example, the DAR may be 3.1, 3.2, 3.3, 3.4, 3.5, 3.6, 3.7, 3.8, 3.9, 7.1, 7.2, 7.3, 7.4, 7.5, 7.6, 7.7, 7.8, 7.9 or any fraction between each integer of 1 to 24.

The linker-payload or antibody-drug conjugate of the present disclosure are useful for treating cancers. In some embodiments, the examples of cancers that can be treated by the linker-payload or antibody-drug conjugate of the present disclosure are breast cancer, gastric cancer, ovarian cancer, lung cancer, colon cancer, liver cancer, brain cancer, prostate cancer, melanoma, pancreatic cancer, head and neck cancer, neuroendocrine neoplasia, but are not limited thereto.

Therefore, the present disclosure also relates to a method for treating a cancer, comprising administering to a subject in need thereof a therapeutically effective amount of a linker-payload or an antibody-drug conjugate of the present disclosure.

The present disclosure further provides a use of a linker-payload or an antibody-drug conjugate of the present disclosure in the manufacture of a medicament for treating a cancer.

The present disclosure further provides a linker-payload or an antibody-drug conjugate of the present disclosure for use as a medicament. In some embodiment, the linker-payload or antibody-drug conjugate is used in the treatment of a cancer.

As used herein, the term “a therapeutically effective amount” is the quantity of an active agent which achieves a clinical outcome when the compound is administered to a subject. For example, when an active agent of the present disclosure is administered to a subject with a cancer, a “clinical outcome” includes reduction in tumor mass, reduction in metastasis, reduction in the severity of the symptoms associated with the cancer and/or increase in the longevity of the subject. The effective amount may vary, as recognized by those skilled in the art, depending on routes of administration, excipient usage, the possibility of co-usage with other therapeutic treatment, and the condition to be treated.

As used herein, the terms “treatment,” “treating” and the like are used herein to generally mean obtaining a desired pharmacologic or physiologic effect. The effect may be prophylactic in terms of completely or partially preventing of alleviating a condition, appearance, disease or symptom and/or may be therapeutic in terms of a partial or complete cure for a condition and/or adverse effect attributable to a condition or disease. The term “treatment” as used herein covers any treatment of a condition, disease or undesirable appearance in a mammal, e.g., a human, and includes: (a) inhibiting the disease, condition or symptom, i.e., causing regression of a condition or symptom; and (b) relieving the disease, condition or symptom, i.e., causing regression of a condition or symptom.

As used herein, “subject” may encompass any vertebrate including, but not limited to, mammals, and/or reptiles. However, advantageously, the subject is a mammal such as a human, or an animal mammal such as a domesticated mammal, e.g., a dog, a cat, a horse, or the like, or a production mammal, e.g., a cow, a sheep, a pig, or the like.

### EXAMPLES

Various properties and efficacies will be illustrated by Examples below. The Examples set forth are used to illustrate the properties of the present disclosure which is not limited to those illustrated in the particular examples.

### Example 1: Preparation of the Linker-Payload of the Present Disclosure

(1) Chemical materials and reagents: chemical materials and reagents used in this example are listed in Table 3, and those commercial reagents and solvents were purchased from Aldrich, Thermo scientific, Uni-onward, Combi-blocks, ACROS, Echo chemical, MORR, BLDpharm, Matrix scientific, AK scientific, and Merck used without further purification.

TABLE 3

| Structure | Name | CAS No. |
|---|---|---|
| 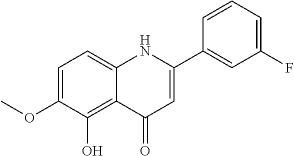 [SMILES: COc1ccc2[nH]c(-c3cccc(F)c3)cc(=O)c2c1O] | CVM-1125, 2-(3- Fluorophenyl)-5- hydroxy-6- methoxyquinolin-4(1H)- one | — |
| 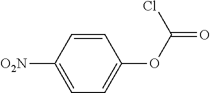 [SMILES: O=C(Cl)Oc1ccc([N+](=O)[O-])cc1] | 4-Nitrophenyl chloroformate | 7693-46-1 |
| 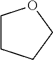 | Tetrahydrofuran | 109-99-9 |
| 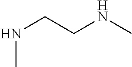 | 1,2-Dimethylethylenediamine | 110-70-3 |
| 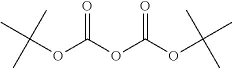 | Di-tert-butyl decarbonate, Boc anhydride | 24424-99-5 |
| CH₂Cl₂ | Dichloromethane | 75-09-2 |
| 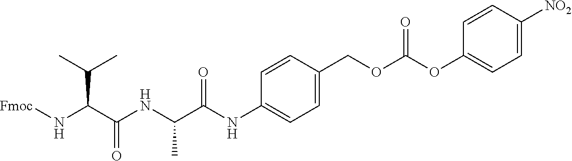 | Fmoc-Val-Ala-PAB-PNP | 1394238-92-6 |
| 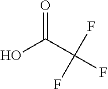 | Trifluoroacetic acid | 76-05-1 |
| 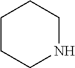 | piperidine | 110-89-4 |
| CH₃OH | Methanol | 67-56-1 |
| 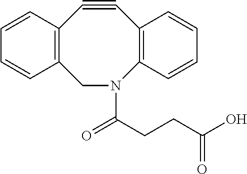 [SMILES: O=C(O)CCC(=O)N1Cc2ccccc2C#Cc2ccccc21] | DBCO-acid | 1353016-70-2 |
| 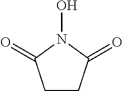 | N-Hydroxysuccinimide | 6066-82-6 |
| 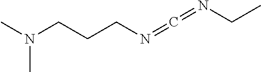 | 1-Ethyl-3-(3- dimethylaminopropyl) carbodiimide | 1892-57-5 |
| 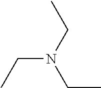 | Triethylamine | 121-44-8 |
| 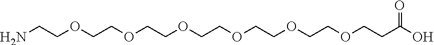 [SMILES: NCCOCCOCCOCCOCCOCCOCCC(=O)O] | 3-(2-(2-(2-(2-(2-(2- Aminoethoxy)ethoxy)ethoxy) ethoxy)ethoxy)ethoxy) propanoic acid | 905954-28-1 |
| 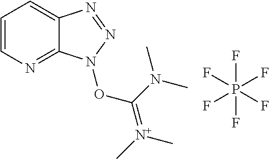 | Hexafluorophosphate Azabenzotriazole Tetramethyl Uronium | 148893-10-1 |
| 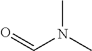 | Dimethylformamide | 68-12-2 |
| 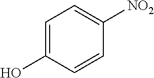 | 4-Nitrophenol | 100-02-7 |
| 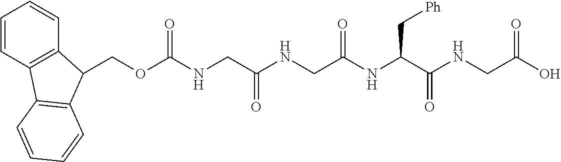 [SMILES: O=C(O)CNC(=O)[C@H](Cc1ccccc1)NC(=O)CNC(=O)CNC(=O)OCC1c2ccccc2-c2ccccc21] | Fomc-Gly-Gly-Phe-Gly-OH | 1817857-75-2 |
| 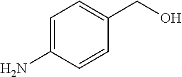 | 4-Aminobenzyl alcohol | 623-04-1 |
| 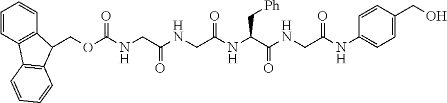 [SMILES: O=C(CNC(=O)OCC1c2ccccc2-c2ccccc21)NCC(=O)N[C@@H](Cc1ccccc1)C(=O)NCC(=O)Nc1ccc(CO)cc1] | Fmoc-Gly-Gly-Phe- Gly-PAB-OH | 2632341-91-2 |
| 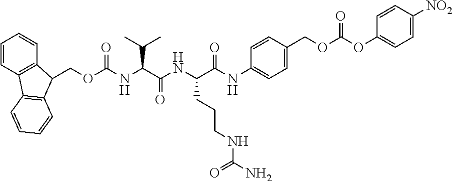 [SMILES: CC(C)[C@H](NC(=O)OCC1c2ccccc2-c2ccccc21)C(=O)N[C@@H](CCCNC(N)=O)C(=O)Nc1ccc(COC(=O)Oc2ccc([N+](=O)[O-])cc2)cc1] | Fmoc-Val-Cit-PAB-PNP | 863971-53-3 |
| 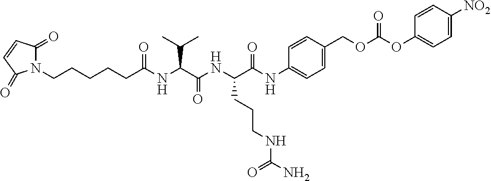 [SMILES: CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1] | Mc-Val-Cit-PABC-PNP | 159857-81-5 |
| 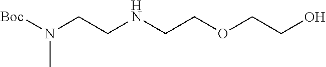 [SMILES: CN(CCNCCOCCO)C(=O)OC(C)(C)C] | Tert-butyl(2-((2-(2- hydroxyethoxy)ethyl)amino) ethyl)(methyl)carbamate | 1345680-97-8 |
| 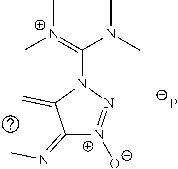 | Hexafluorophosphate azabenzotriazole tetramethyl uranium (HATU) | 148893-10-1 |

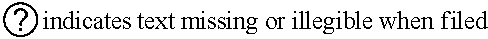

(2) Chemical equipment: 1H-NMR spectra were recorded on Nuclear Magnetic Resonance (NMR). & value is presented in ppm relative to TMS as an internal standard (0 ppm). Multiplicities were recorded as singlet(s), doublet (d), triplet (t), quartet (q), double of doublet (dd), and broadened (br). Coupling constants (J) are expressed in Hz. Mass spectra (MS) were measured by LC-MS/MS System. Spectral data are recorded as m/z values.

In this Example, the linker-payload of DBCO-PEG6-va-PAB-DMEA-CVM-1125 for the present disclosure, compound 15 was prepared by the following Scheme 1.

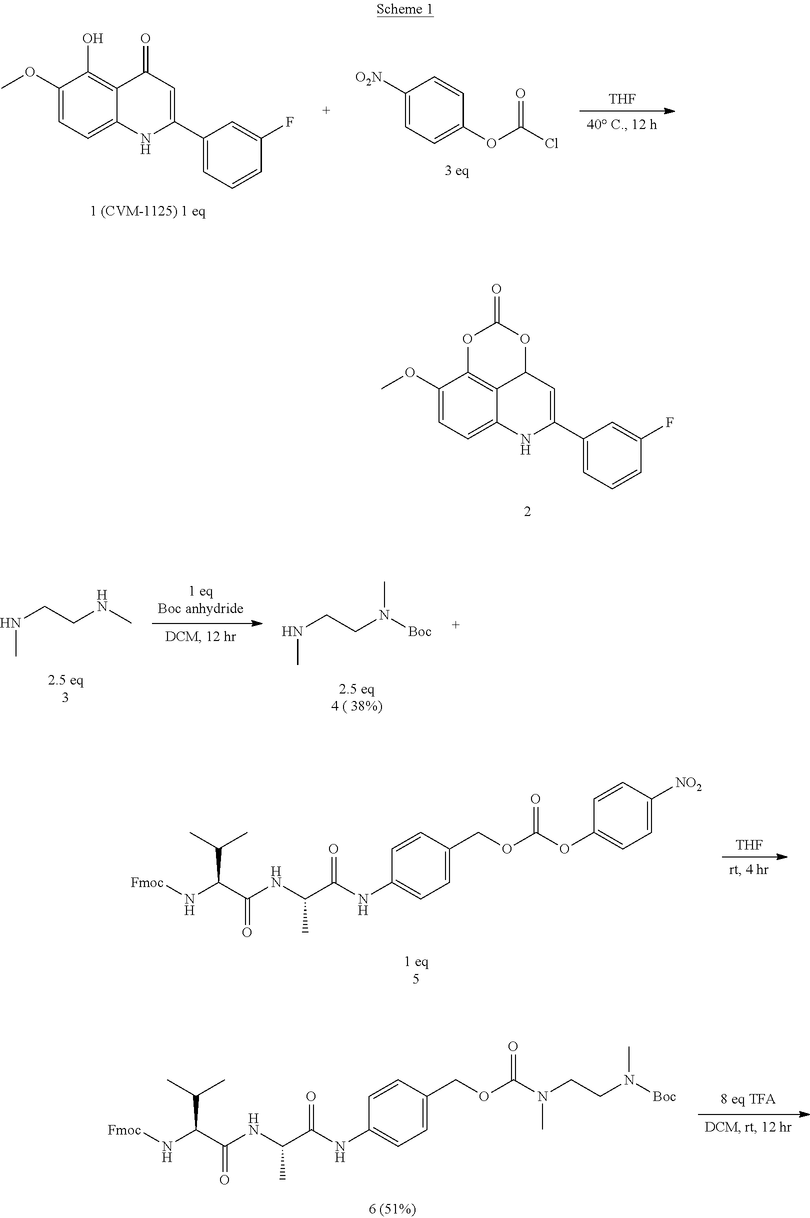

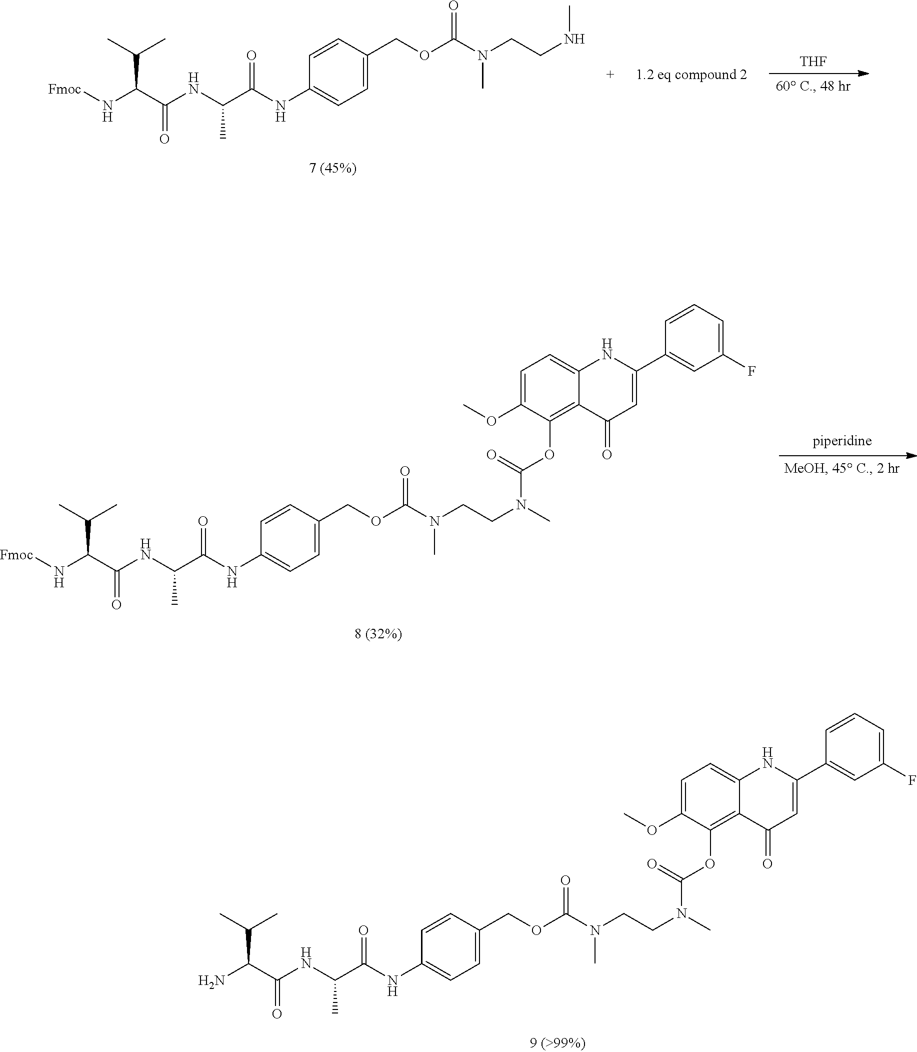

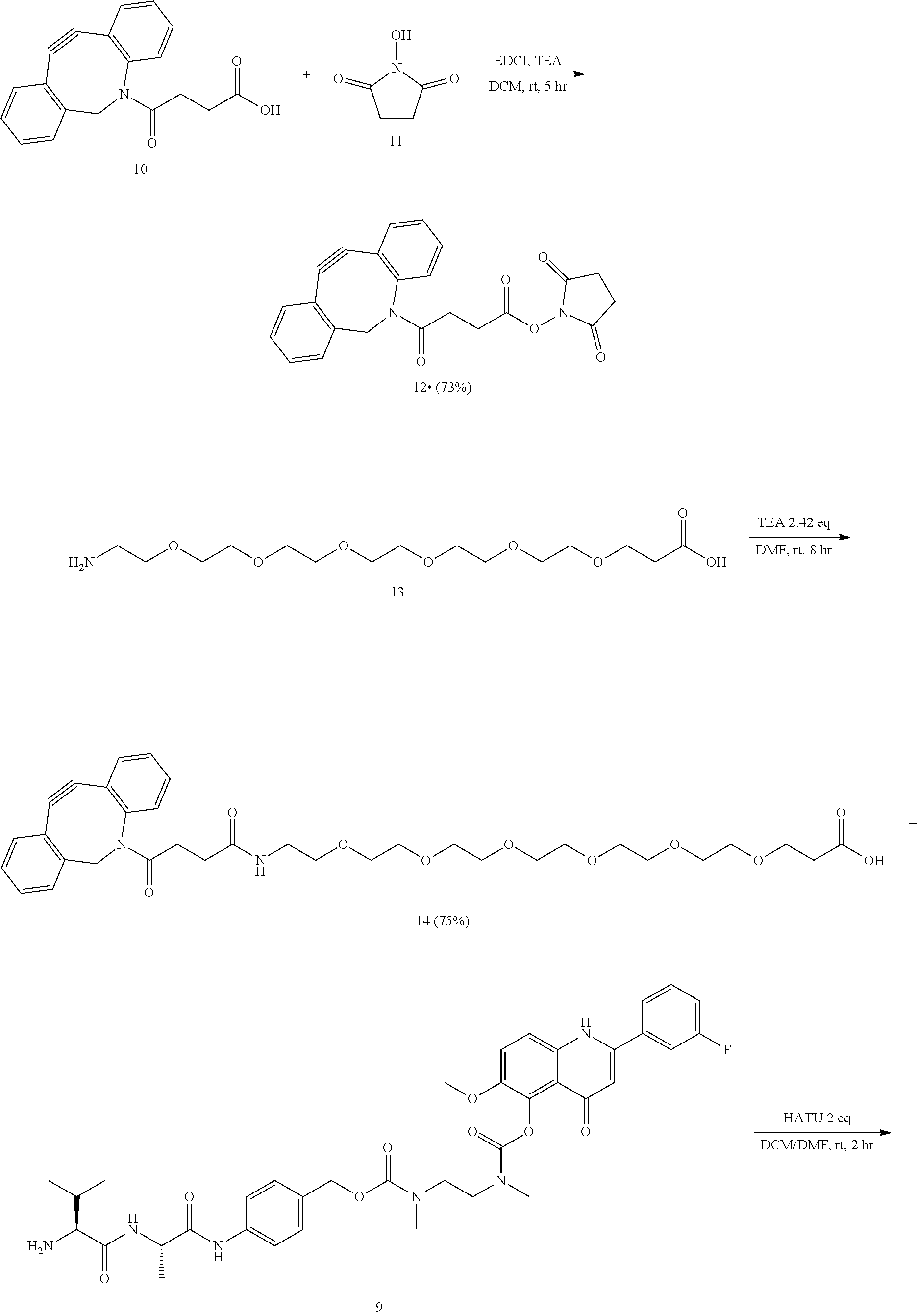

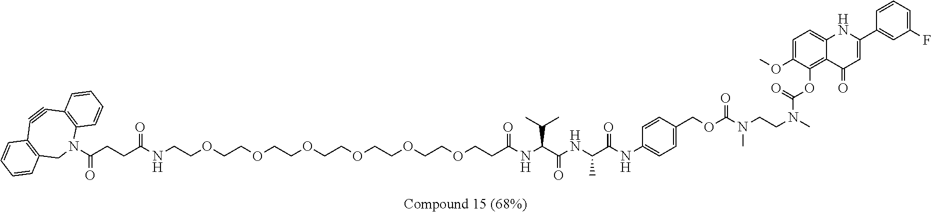 [SMILES: COc1ccc2[nH]c(-c3cccc(F)c3)cc(=O)c2c1OC(=O)N(C)CCN(C)C(=O)OCc1ccc(NC(=O)[C@H](C)NC(=O)[C@@H](NC(=O)CCOCCOCCOCCOCCOCCOCCNC(=O)CCC(=O)N2Cc3ccccc3C#Cc3ccccc32)C(C)C)cc1]

### Synthesis of Compound 2, (5-(3-fluorophenyl)-9-methoxy-3a,6-dihydro-[1,3]dioxino [4,5,6-de]quinolin-2-one), the Activation Step

In a 20 mL sample vial, CVM-1125 (100 mg, 0.37 mmol, compound 1) was dissolved in THF (16 mL). To this solution, 4-nitrobenzyl chloroformate (223.7 mg, 1.11 mmol, the activation reagent) was added and the reaction mixture was stirred at 45° C. for 12 hours. After vacuum filtration, the resulting yellow solid compound 2 (28.5 mg, 25% yield) was obtained. ¹H NMR (CD₃OD, 400 MHZ): δ 8.13 (d, J=9.7 Hz, 1H), 8.07 (d, J=9.4 Hz, 1H), 7.81-7.71 (m, 3H), 7.53 (t, J=7.8 Hz, 3H), 7.20 (s, 1H), 4.08 (s, 3H).

### Synthesis of Compound 4. (tert-butyl methyl(2-(methylamino)ethyl)carbamate)

N, N-dimethylethyldiamine (1.28 g, 14.53 mmol, compound 3) was added to a reaction flask and dissolved in DCM (8.0 mL) at 0° C. A mixture of Boc anhydride (1.27 g, 5.81 mmol) and DCM (3 mL) was then slowly added dropwise to the reaction flask. After complete addition, the reaction mixture was returned to room temperature and stirred for 12 hours. After the reaction was completed, the reaction was quenched with water (5.0 mL) and extracted with DCM (10 mL). The resulting organic solvent was concentrated under vacuum, and the crude product was purified by column chromatography (DCM/MeOH: 20/1) to yield the compound 4 (415.5 mg, 38% yield). ¹H NMR (CDCl₃, 400 MHZ): § 3.34 (br, 2H), 2.88 (s, 3H), 2.74 (br, 2H), 2.46 (s, 3H), 1.46 (s, 9H).

### Synthesis of Compound 6, (4-((S)-2-((S)-2-((((9H-fluoren-9-yl)methoxy)carbonyl)amino)-3-methylbutanamido)propanamido)benzyl tert-butyl ethane-1,2-diylbis(methylcarbamate))

In THF (5 mL), Fmoc-Val-Ala-PAB-PNP(601.74 mg, 0.884 mmol, compound 5) was dissolved, followed by the addition of tert-butyl methyl(2-(methylamino)ethyl)carbamate (415 mg, 2.21 mmol) to form a reaction mixture. The reaction mixture was stirred at room temperature for 4 hours, and then concentrated to yield a yellow solid. The crude product was purified by column chromatography (DCM/MeOH: 20/1) to afford the compound 6 (320 mg, 51% yield). ¹H NMR (d₆-DMSO, 400 MHZ): § 10.01 (br, 1H) 8.18 (d, J=6.9 Hz, 1H), 7.89 (d, J=7.5 Hz, 2H), 7.74 (t, J=7.0 Hz, 2H), 7.57 (d, J=8.3 Hz, 2H), 7.46-7.37 (m, 3H), 7.35-7.24 (m, 4H), 4.97 (br, 2H), 4.46-4.37 (m, 1H), 4.34-4.18 (m, 3H), 3.91 (t, J=7.2 Hz, 1H), 2.87-2.80 (br, 3H), 2.76 (br, 1H), 2.68 (br, 2H), 2.04-1.94 (m, 1H), 1.35 (s, 9H), 1.30 (d, J=7.1 Hz, 3H), 0.87 (dd, J=12.4, 6.8 Hz, 6H).

### Synthesis of Compound 7, (4-((S)-2-((S)-2-((((9H-fluoren-9-yl)methoxy)carbonyl)amino)-3-methylbutanamido)propanamido)benzyl methyl(2-(methylamino)ethyl)carbamate)

Compound 6 (483.2 mg, 0.67 mmol) was added to a reaction flask and dissolved in DCM (8 mL). TFA (0.41 mL, 5.40 mmol) was then slowly added dropwise to the reaction mixture in the reaction flask at 0° C. The reaction was allowed to warm to room temperature and stirred for 12 hours. After completion, the reaction mixture was concentrated under vacuum. Purification by column chromatography (DCM/MeOH: 50/1 to 20/1) afforded the compound 7 (187.2 mg, 45% yield). ¹H NMR (CD₃OD, 400 MHZ): § 9.78 (br, 1H), 8.37 (d, J=6.6 Hz, 1H), 7.83 (d, J=7.7 Hz, 2H), 7.68 (dd, J=7.3, 3.2 Hz, 2H), 7.62 (d, J=8.3 Hz, 2H), 7.46-7.28 (m, 6H), 5.13 (s, 2H), 4.57-4.46 (m, 1H), 7.46-7.37 (m, 2H), 4.25 (t, J=6.7 Hz, 1H), 4.00-3.93 (m, 1H), 3.63 (t, J=5.6 Hz, 2H), 3.26-3.12, (m, 2H), 3.00 (s, 3H), 2.78-2.64 (m, 3H), 2.18-2.03 (m, 1H), 1.47 (d, J=7.2 Hz, 3H), 1.00 (dd, J=9.3, 6.9 Hz, 6H).

### Synthesis of Compound 8, (4-((S)-2-((S)-2-((((9H-fluoren-9-yl)methoxy)carbonyl)amino)-3-methylbutanamido)propanamido)benzyl(2-(3-fluorophenyl)-6-methoxy-4-oxo-1,4-dihydroquinolin-5-yl)ethane-1,2-diylbis(methylcarbamate))

Compound 7 (65.48 mg, 0.103 mmol) and compound 2 (35.6 mg, 0.114 mmol), were dissolved in THF (10.0 mL) and placed in a reaction flask. The reaction mixture including compound 7 and compound 2 was heated to 50° C. and allowed to react for two days. After completion of the reaction, the solvent was evaporated under vacuum. Purification of the resulting mixture was achieved through column chromatography (DCM/MeOH: 50/1 to 10/1), yielding the compound 8 (31.4 mg, 32% yield). ¹H NMR (CD₃OD, 400 MHZ) δ 7.80-7.72 (m, 2H), 7.68-7.65 (m, 16H), 6.34-6.28 (br, 1H), 5.20-4.80 (m, 2H), 4.55-4.28 (m, 3H), 4.25-4.10 (m, 1H), 4.05-3.92 (m, 1H), 3.87-3.65 (m, 5H), 3.12-2.90 (m, 6H), 2.20-2.00 (m, 1H), 1.47-1.35 (m, 3H), 1.07-0.85 (m, 6H).

### Synthesis of Compound 9, (4-((S)-2-((S)-2-amino-3-methylbutanamido)propanamido)benzyl(2-(3-fluorophenyl)-6-methoxy-4-oxo-1,4-dihydroquinolin-5-yl)ethane-1,2-diylbis(methylcarbamate))

Compound 8 (31.4 mg, 0.033 mmol) was dissolved in methanol (3.0 mL), followed by the addition of piperidine (0.1 mL) for reaction. The reaction temperature was then raised to 45° C. and allowed to react for 2 hours. After completion, the solvent was evaporated under vacuum. Purification of the resulting mixture was achieved through column chromatography (DCM/MeOH: 30/1 to 10/1), yielding the compound 9 (23.7 mg, >99% yield). ¹H NMR (CD₃OD, 400 MHZ): δ 7.72-7.22 (m, 10H), 6.35 (br, 1H), 5.23-5.03 (m, 2H), 4.62 (br, 1H), 4.56-4.47 (m, 1H), 3.93-3.39 (m, 6H), 3.29-2.96 (m, 7H), 2.04 (br, 1H), 1.51-1.40 (m, 3H), 1.07-0.92 (m, 6H).

### Synthesis of Compound 12, (Dibenzocyclooctyne-N-hydroxysuccinimidyl Ester)

DBCO-acid (1.5 g, 5 mmol, compound 10), N-hydroxysuccinimide (0.63 mg, 5.5 mmol, compound 11), EDCI (1.1 g, 5.5 mmol), and triethylamine (0.75 mL, 5.5 mmol) were added to a reaction flask. The mixture in the reaction flask was allowed to react at room temperature for 5 hours. Afterward, 2% HCl was added to quench the reaction. The resulting mixture was then extracted twice with DCM, and the organic layers were combined. After removal of water by concentration, purification of the crude product was performed by column chromatography (hexane/ethyl acetate: 4/1 to 1/1), yielding the compound 12 (1.4402 g, 73% yield). ¹H NMR (CDCl₃, 400 MHZ) & 7.69 (d, J=7.1 Hz, 1H), 7.45-7.35 (m, 5H), 7.31 (ddd, J=7.5, 7.5, 1.1 Hz, 1H), 7.27-7.24 (m, 2H), 5.18 (d, J=13.9 Hz, 1H), 3.69 (d, J=13.9 Hz, 1H), 2.98 (dt, J=17.3, 7.6 Hz, 1H), 2.86-2.74 (m, 5H), 2.64 (ddd, J=17.3, 7.6, 5.4 Hz, 1H), 2.08 (ddd, J=17.3, 7.6, 5.4 Hz, 1H).

### Synthesis of Compound 14, (Dibenzocyclooctyne-PEG6-propionic acid)

Compound 12 (300 mg, 0.746 mmol), compound 13 (316 mg, 0.895 mmol), and triethylamine (0.25 mL, 1.805 mmol) were added to a reaction flask. The mixture in the reaction flask was allowed to react at room temperature for 8 hours. After concentration, the crude product was purified by column chromatography (DCM/MeOH: 20/1 to pure MeOH), yielding the compound 14 (360.6 mg, 75% yield). ¹H NMR (CD₃OD, 400 MHZ) δ 7.73 (d, J=7.0 Hz, 1H), 7.70-7.65 (m, 1H), 7.58-7.52 (m, 3H), 7.45 (ddd, J=7.4, 7.4, 1.5 Hz, 1H), 7.41 (ddd, J=7.4, 7.4, 1.5 Hz, 1H), 7.33 (dd, J=7.4, 1.5 Hz), 5.20 (d, J=14.0 Hz, 1H), 3.80 (t, J=6.2 Hz, 2H), 3.78-3.64 (m, 22H), 3.58-3.48 (m, 2H), 3.34 (t, J=5.6 Hz, 2H), 2.84-2.72 (m, 1H), 2.59 (t, J=6.2 Hz, 2H), 2.49-2.39 (m, 1H), 2.32-2.22 (m, 1H), 2.12-2.02 (m, 1H).

### Synthesis of Compound 15, the Linker-Payload of the Present Disclosure (DBCO-PEG6-va-PAB-DMEA-CVM-1125)

Compound 14 (16 mg, 0.024 mmol) and compound 9 (16 mg, 0.022 mmol) were dissolved in DCM (2.0 mL) and DMF (1 mL), and followed by the addition of HATU (17 mg, 0.044 mmol) to form a mixture. The mixture was stirred at room temperature for 2 hours, then concentrated and dried. The crude product was purified by column chromatography (DCM/MeOH: 20/1), yielding the compound 15 (20.0 mg, 68% yield). ¹H NMR (CD₃OD, 400 MHZ): § 7.71-7.50 (m, 9H), 7.49-7.45 (m, 2H), 7.41 (br, 1H), 7.38-7.29 (m, 4H), 7.29-7.19 (m, 2H), 6.36 (br, 1H), 5.21-5.01 (m, 4H), 4.57-4.46 (m, 1H), 4.28-4.18 (m, 1H), 3.93-3.66 (m, 10H), 3.66-3.52 (m, 23H), 3.47-3.39 (m, 1H), 3.27-3.20 (m, 2H), 3.15-2.96 (m, 6H), 2.77-2.65 (m, 1H), 2.58 (br, 2H), 2.37 (dt, J=15.2, 7.4 Hz, 1H), 2.24-2.08 (m, 2H), 2.06-1.93 (m, 1H), 1.50-1.40 (br, 4H), 1.09-0.96 (m, 7H).

In this Example, another cleavable linker, GGFG, was used to replace va linker. The linker-payload of DBCO-PEG6-GGFG-PAB-DMEA-CVM-1125 for the present disclosure, compound 26 was prepared by the following Scheme 2.

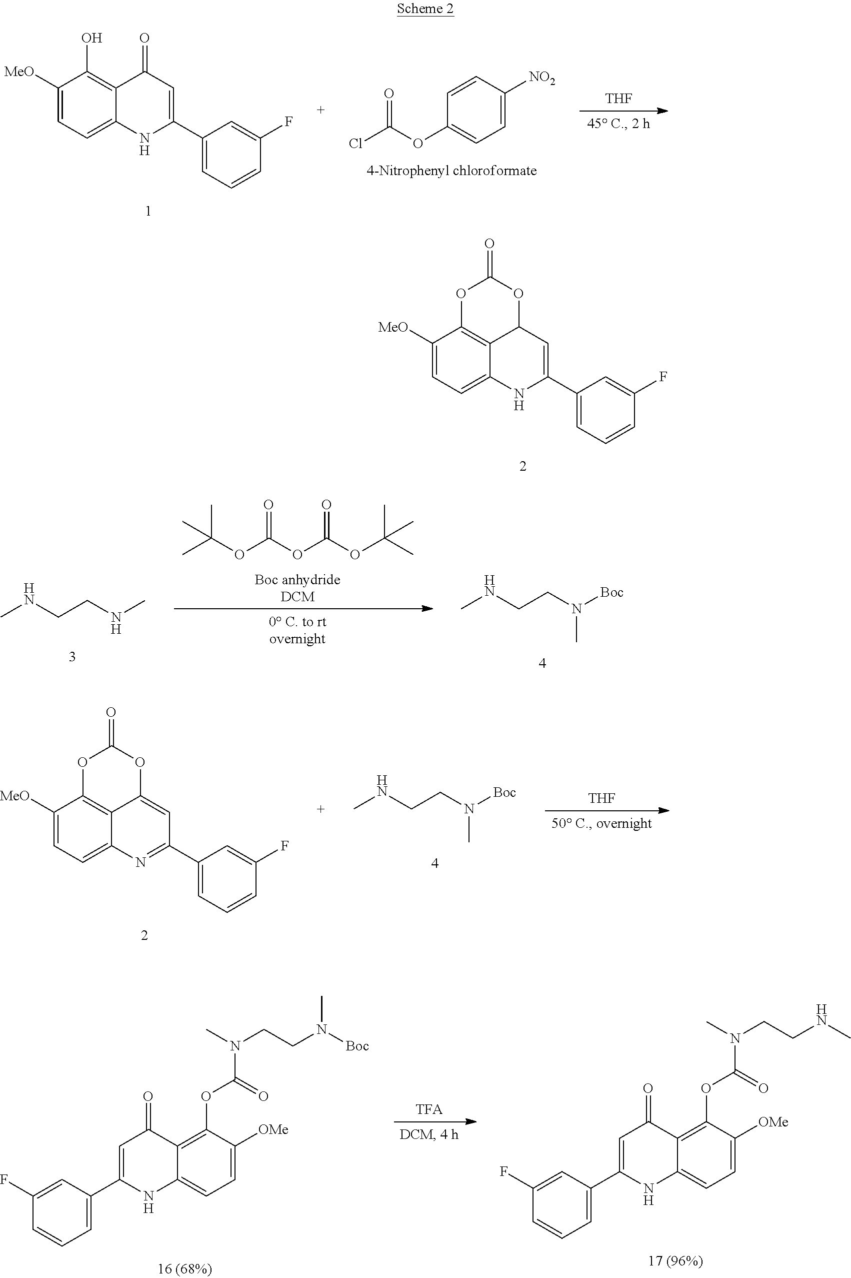

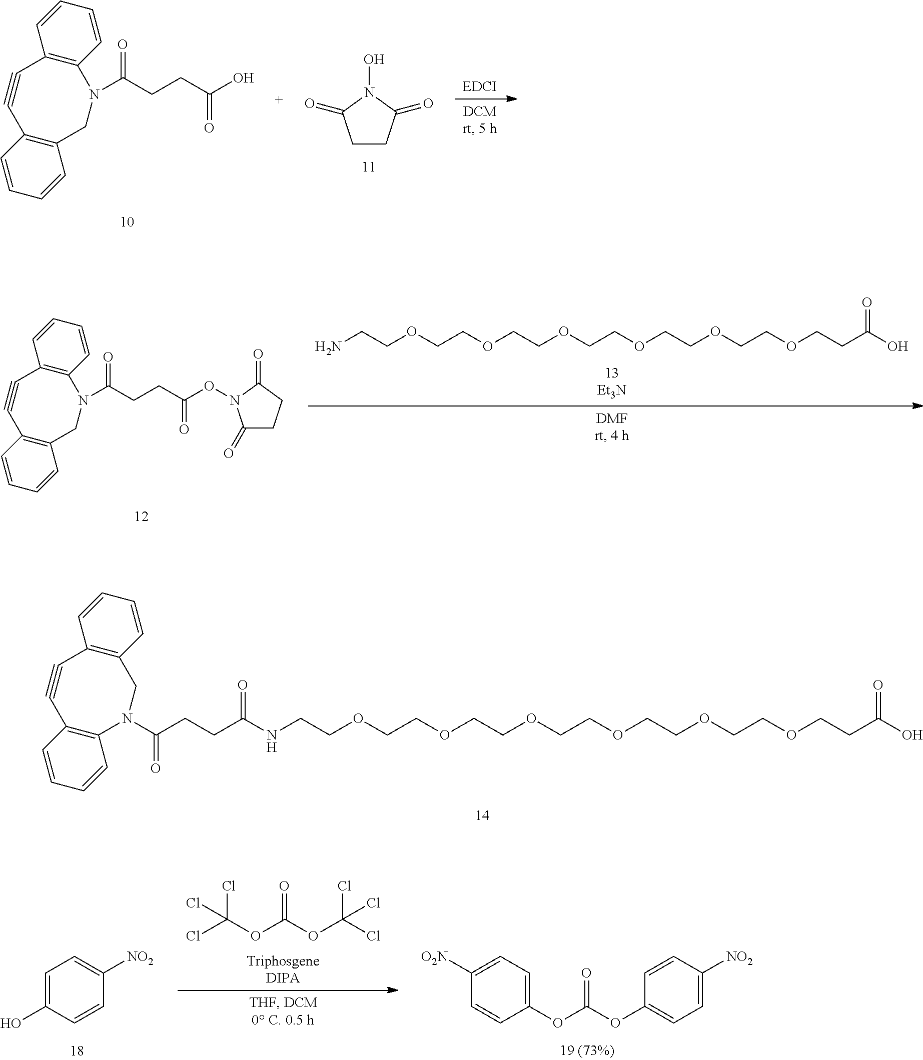

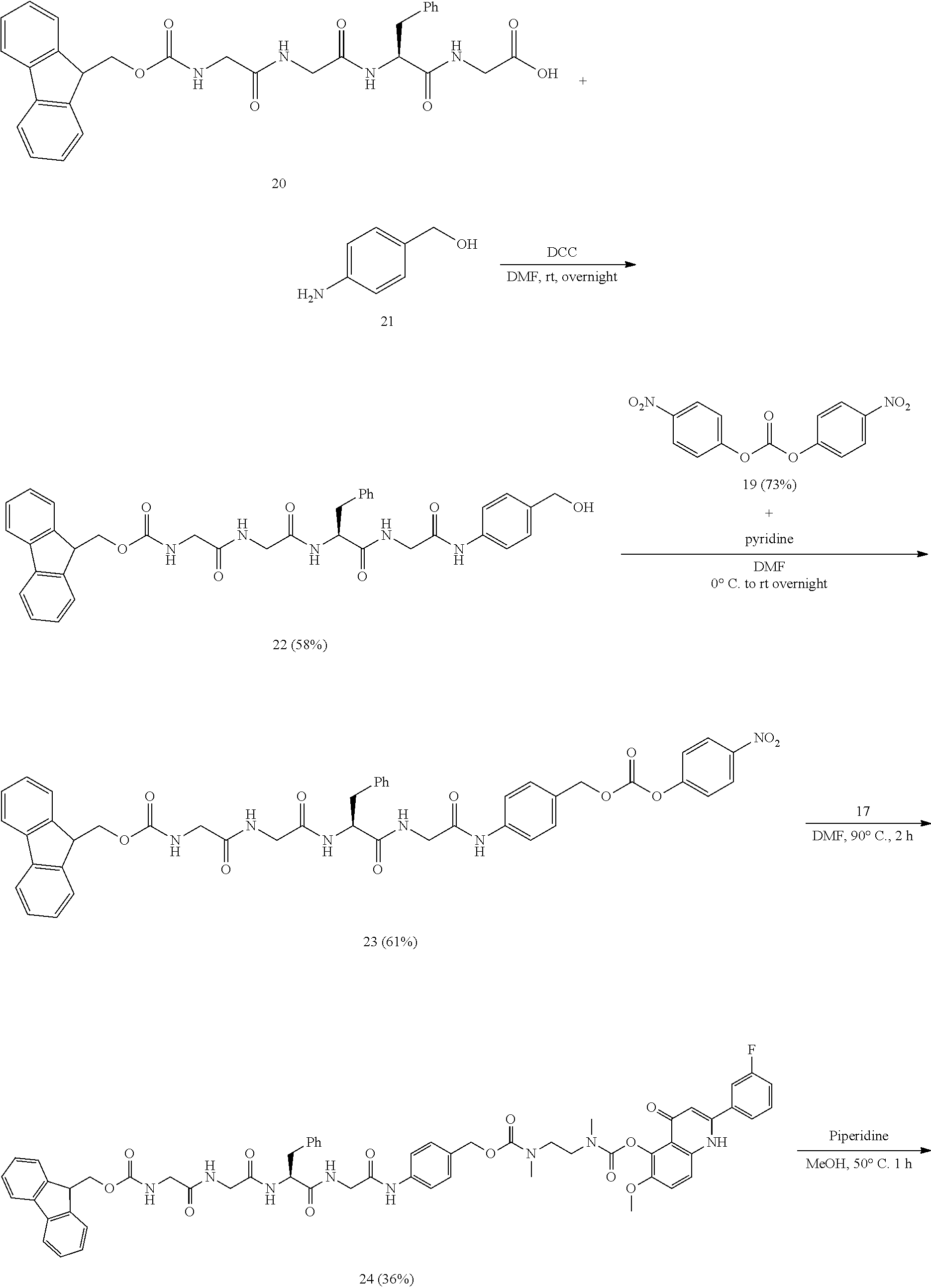

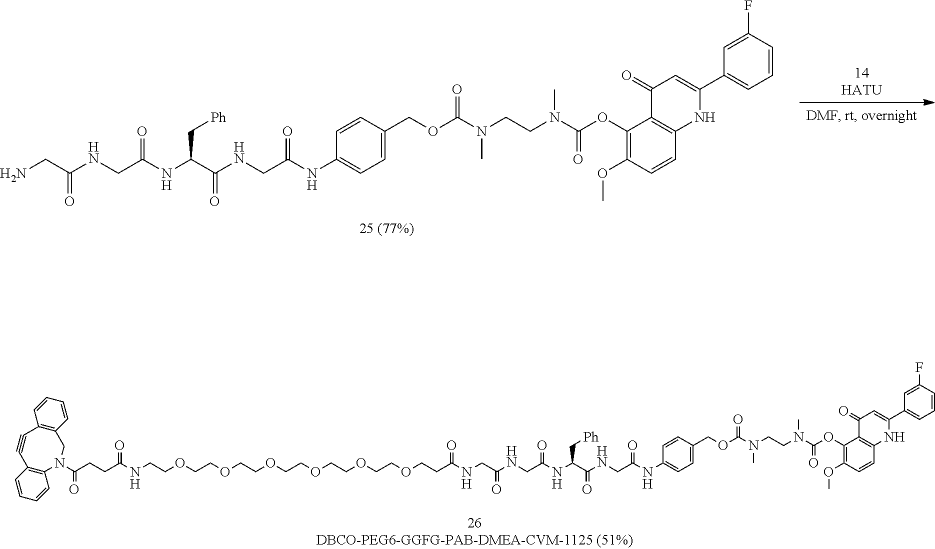 [SMILES: COc1ccc2[nH]c(-c3cccc(F)c3)cc(=O)c2c1OC(=O)N(C)CCN(C)C(=O)OCc1ccc(NC(=O)CNC(=O)[C@H](Cc2ccccc2)NC(=O)CNC(=O)CN)cc1 ; "14HATU" ; "DMF, rt, overnight" ; COc1ccc2[nH]c(-c3cccc(F)c3)cc(=O)c2c1OC(=O)N(C)CCN(C)C(=O)OCc1ccc(NC(=O)CNC(=O)[C@H](Cc2ccccc2)NC(=O)CNC(=O)CNC(=O)CCOCCOCCOCCOCCOCCOCCNC(=O)CCC(=O)N2Cc3ccccc3C#Cc3ccccc32)cc1]

### Synthesis of Compound 16 (tert-butyl(2-(3-fluorophenyl)-6-methoxy-4-oxo-1,4-dihydroquinolin-5-yl)ethane-1,2-diylbis(methylcarbamate))

Compound 2 (0.96 mmol, 1.0 equiv.) was dissolved in THF (40 mL), followed by the addition of compound 4 (1.16 mmol, 1.2 equiv.). The reaction mixture was stirred at 50° C. overnight. After completion of the reaction, the solution was evaporated under vacuum. Purification of the resulting mixture was achieved through column chromatography (DCM/MeOH: 40/1 to 20/1) to yield compound 16 (yellow powder, 68% yield). ¹H NMR (CD₃OD, 400 MHZ): § 1.49 (d, J=22.1 Hz, 9H), 2.94-3.06 (m, 3H), 3.10 (s, 2H), 3.26 (s, 1H), 3.37-4.46 (m, 1H), 3.55-3.76 (m, 2H), 3.77-3.88 (m, 3H), 3.92-4.17 (m, 1H), 6.28 (s, 1H), 7.24-7.35 (m, 1H), 7.36-7.61 (m, 5H).

### Synthesis of Compound 17 (2-(3-fluorophenyl)-6-methoxy-4-oxo-1,4-dihydroquinolin-5-yl methyl(2-(methylamino)ethyl)carbamate)

Compound 16 (0.51 mmol, 1.0 equiv.) was dissolved in DCM (5 mL) at 0° C., followed by the dropwise addition of trifluoroacetic acid (15.3 mmol, 30.0 equiv.). After complete addition, the reaction mixture was returned to room temperature and stirred overnight. After completion of the reaction, the resulting solution was concentrated under vacuum, and the crude product was purified by column chromatography (DCM/MeOH: 50/1 to 5/1) to yield compound 17 (yellow powder, 96% yield). 1H NMR (CD₃OD, 400 MHZ): § 2.81 (d, J=18.8 Hz, 3H), 3.14 (s, 2H), 3.25 (s, 1H), 3.40-3.71 (m, 2H), 3.86 (d, J=9.3 Hz, 3H), 4.11-4.44 (m, 1H), 6.46 (d, J=7.8 Hz, 1H), 7.26-7.34 (m, 1H), 7.50-7.71 (m, 5H).

### Synthesis of Compound 19 (Bis-(4-nitrophenyl)carbonate)

Compound 18 (7.2 mmol, 1.0 equiv.) was added to a reaction flask and dissolved in THF (40 mL), followed by the addition of DIPA (25.2 mmol, 3.5 equiv.). A mixture of triphosgene (5.04 mmol, 0.7 equiv.) and DCM (5 mL) was then slowly added dropwise to the reaction flask over a period of 10 minutes at 0° C. The reaction mixture was stirred at 0° C. for 30 minutes. After the reaction was completed, the reaction was quenched with 1N HCl and extracted with DCM. The resulting solution was concentrated under vacuum, and the crude product was purified by column chromatography (pure DCM). The purified solution was treated with hexane to precipitate compound 19 (white solid, 73% yield). ¹H NMR (CDCl₃, 400 MHZ): δ 7.51 (d, J=12.0 Hz, 2H), 8.35 (d, J=8.0 Hz, 2H).

### Synthesis of Compound 22 ((9H-fluoren-9-yl)methyl(S)-(2-((2-((1-((2-((4-(hydroxymethyl)phenyl)amino)-2-oxoethyl)amino)-1-oxo-3-phenylpropan-2-yl)amino)-2-oxoethyl)amino)-2-oxoethyl)carbamate)

Compound 20 (0.91 mmol, 1.0 equiv.), DCC (1.36 mmol, 1.5 equiv.), and compound 21 (1.36 mmol, 1.5 equiv.) were dissolved in DMF (5 mL). The reaction mixture was stirred at room temperature for overnight. After completion of the reaction, the resulting solution was concentrated under vacuum, and the crude product was purified by column chromatography (DCM/MeOH: 50/1 to 5/1) to yield the compound 22 (brown powder, 58% yield). ¹H NMR (CD₃OD, 400 MHZ): § 3.06 (dd, J=13.9, 9.3 Hz, 1H), 3.26 (dd, J=13.8, 5.6 Hz, 1H), 3.78-3.82 (m, 3H), 3.88 (d, J=16.8 Hz, 1H), 3.95 (d, J=16.8 Hz, 1H), 4.05 (d, J=16.8 Hz, 1H), 4.23 (t, J=6.8 Hz, 1H), 4.37-4.45 (m, 2H), 4.56-4.59 (m, 3H), 7.20-7.24 (m, 1H), 7.28-7.33 (m, 8H), 7.40 (t, J=7.5 Hz, 2H), 7.61 (d, J=8.5 Hz, 2H), 7.67 (d, J=7.1 Hz, 2H), 7.81 (d, J=7.6 Hz, 2H).

### Synthesis of Compound 23 ((9H-fluoren-9-yl)methyl(S)-(2-((2-((1-((2-((4-((((4-nitrophenoxy)carbonyl)oxy)methyl)phenyl)amino)-2-oxoethyl)amino)-1-oxo-3-phenylpropan-2-yl)amino)-2-oxoethyl)amino)-2-oxoethyl)carbamate)

Compound 22 (1.27 mmol, 3.0 equiv.) was dissolved in DMF (3 mL), and pyridine (0.21 mmol, 0.5 equiv.) was added to the solution at 0° C. with stirring for 2 minutes. A mixture of compound 19 and DMF (5 mL) was then slowly added dropwise to the reaction mixture over a period of 10 minutes at 0° C. After allowing the reaction to warm to room temperature, pyridine (0.21 mmol, 0.5 equiv.) was added again, and the mixture was stirred at room temperature for overnight. After completion of the reaction, the resulting solution was concentrated under vacuum, and the crude product was purified by column chromatography (DCM/MeOH: 60/1 to 20/1) to yield compound 23 (white solid, 61% yield). ¹H NMR (d₆-DMSO, 400 MHZ): § 2.80 (dd, J=13.9, 10.1 Hz, 1H), 3.06 (dd, J=13.6, 4.7 Hz, 1H), 3.55-3.70 (m, 3H), 3.77 (dd, J=17.2, 5.9 Hz, 1H), 3.87 (dd, J=12.9, 5.6 Hz, 1H), 3.91 (dd, J=16.4, 5.8 Hz, 1H), 4.19 (t, J=6.8 Hz, 1H), 4.26 (d, J=6.7 Hz, 2H), 4.44-4.55 (m, 1H), 5.22 (s, 1H), 7.16 (q, J=4.2 Hz, 1H), 7.23 (d, J=4.3 Hz, 4H), 7.29 (t, J=7.5 Hz, 2H), 7.34-7.44 (m, 4H), 7.50-7.60 (m, 3H), 7.66 (dd, J=12.3, 7.6 Hz, 4H), 7.86 (d, J=7.5 Hz, 2H), 8.03 (t, J=5.4 Hz, 1H), 8.18 (d, J=7.9 Hz, 1H), 8.28 (d, J=9.2 Hz, 2H), 8.41 (t, J=4.8 Hz, 1H), 9.92 (s, 1H).

### Synthesis of Compound 24 ((S)-4-(11-benzyl-1-(9H-fluoren-9-yl)-3,6,9,12-tetraoxo-2-oxa-4,7,10,13-tetraazapentadecan-15-amido)benzyl(2-(3-fluorophenyl)-6-methoxy-4-oxo-1,4-dihydroquinolin-5-yl)ethane-1,2-diylbis(methylcarbamate))

Compound 23 (0.32 mmol, 1.0 equiv.) and compound 17 (0.80 mmol, 2.5 equiv.) were dissolved in DMF (2 mL). The reaction mixture was stirred at 90° C. for 2 hours. After completion of the reaction, the resulting solution was concentrated under vacuum, and the crude product was purified by column chromatography (DCM/MeOH: 50/1 to 10/1) to yield compound 24 (brown oil, 36% yield). ¹H NMR (CD₃OD, 400 MHz): 2.94-3.14 (m, 6H), 3.18-3.30 (m, 2H), 3.64-3.74 (m, 1H), 3.74-3.85 (m, 6H), 3.85-3.97 (m, 1H), 4.19 (s, 1H), 4.31-4.43 (m, 2H), 4.50-4.59 (m, 1H), 4.98-5.18 (m, 2H), 6.33 (s, 1H), 7.21 (s, 2H), 7.24-7.34 (m, 8H), 7.37 (t, J=7.5 Hz, 3H), 7.46-7.69 (m, 8H), 7.78 (d, J=7.5 Hz, 2H).

### Synthesis of Compound 25 ((S)-4-(2-(2-(2-(2-aminoacetamido)acetamido)-3-phenylpropanamido)acetamido)benzyl(2-(3-fluorophenyl)-6-methoxy-4-oxo-1,4-dihydroquinolin-5-yl)ethane-1,2-diylbis(methylcarbamate))

Compound 24 (0.12 mmol, 1.0 equiv.) was dissolved in MeOH (4 mL), followed by the addition of piperidine (0.5 mL). The reaction mixture was stirred at 50° C. for 1 hour. After completion of the reaction, the resulting solution was concentrated under vacuum, and the crude product was purified by column chromatography (DCM/MeOH: 30/1 to 4/1) to yield compound 25 (brown powder, 77% yield). ¹H NMR (CD₃OD, 400 MHZ): 2.93-3.06 (m, 5H), 3.08-3.15 (m, 1H), 3.18-3.29 (m, 4H), 3.38-3.52 (br, 1H), 3.59-3.96 (m, 8H), 3.98-4.10 (m, 1H), 4.57 (s, 2H), 4.98-5.16 (m, 2H), 6.32 (s, 1H), 7.18-7.45 (m, 9H), 7.43-7.64 (m, 6H).

### Synthesis of Compound 26 (DBCO-PEG6-GGFG-PAB-DMEA-CVM-1125)

Compound 25 (0.09 mmol, 1.0 equiv.), compound 14 (0.10 mmol, 1.1 equiv.) and HATU (0.18 mmol, 2.0 equiv.) were dissolved in DMF (2 mL). The reaction mixture was stirred at room temperature for overnight. After completion of the reaction, the resulting solution was concentrated under vacuum, and the crude product was purified by column chromatography (DCM/MeOH: 40/1 to 5/1) to yield compound 26 (brown oil, 51% yield). ¹H NMR (CD₃OD, 400 MHZ): 1.92-2.02 (m, 1H), 2.10-2.23 (m, 1H), 2.29-2.42 (m, 1H), 2.45-2.58 (m, 2H), 2.64-2.74 (m, 1H), 2.95-3.15 (m, 7H), 3.19-3.30 (m, 3H), 3.36-3.47 (m, 3H), 3.53-3.63 (m, 18H), 3.64-3.75 (m, 4H), 3.76-3.94 (m, 10H), 3.95-4.10 (m, 2H), 4.57 (s, 1H), 4.61 (s, 1H), 4.99-5.21 (m, 3H), 6.33 (s, 1H), 7.17-7.25 (m, 3H), 7.25-7.42 (m, 10H), 7.42-7.47 (m, 2H), 7.48-7.68 (m, 8H).

In this Example, the linker-payload of DBCO-PEG6-GGFG-PAB-CVM-1125 for the present disclosure, compound 29 was prepared by the following Scheme 3.

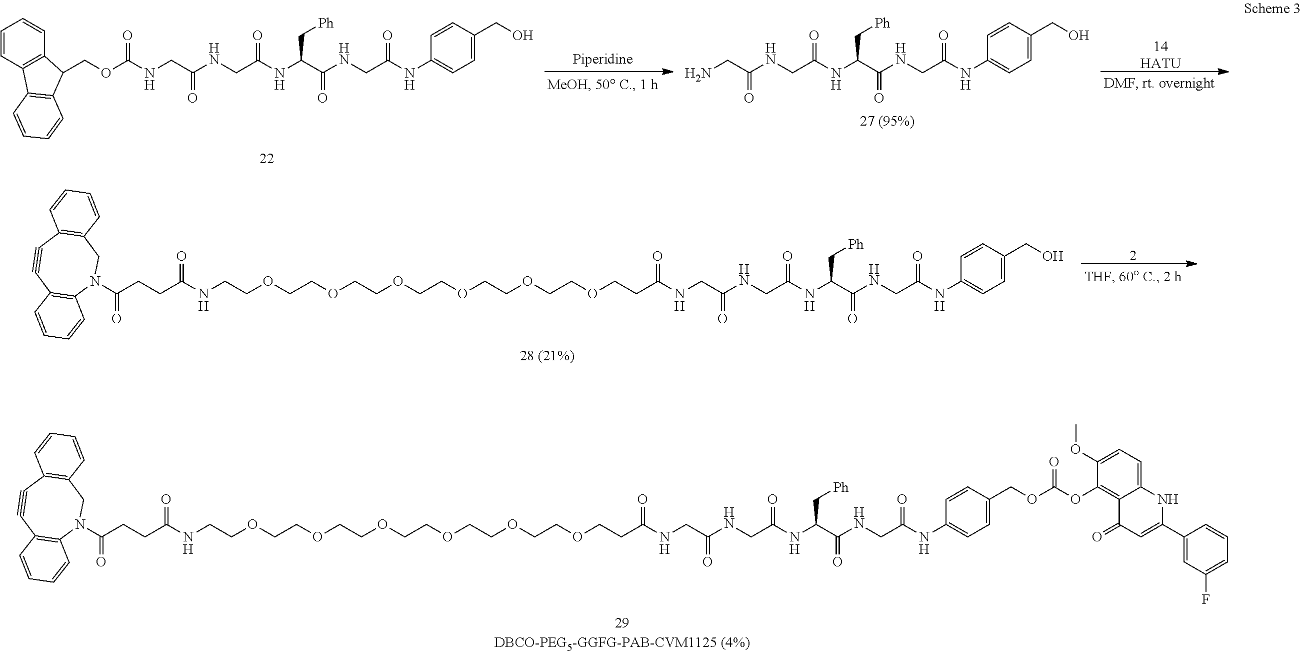 [SMILES: O=C(CNC(=O)OCC1c2ccccc2-c2ccccc21)NCC(=O)N[C@@H](Cc1ccccc1)C(=O)NCC(=O)Nc1ccc(CO)cc1 ; NCC(=O)NCC(=O)N[C@@H](Cc1ccccc1)C(=O)NCC(=O)Nc1ccc(CO)cc1 ; "14HATU" ; O=C(CCC(=O)N1Cc2ccccc2C#Cc2ccccc21)NCCOCCOCCOCCOCCOCCOCCC(=O)NCC(=O)NCC(=O)N[C@@H](Cc1ccccc1)C(=O)NCC(=O)Nc1ccc(CO)cc1 ; "2" ; COc1ccc2[nH]c(-c3cccc(F)c3)cc(=O)c2c1OC(=O)OCc1ccc(NC(=O)CNC(=O)[C@H](Cc2ccccc2)NC(=O)CNC(=O)CNC(=O)CCOCCOCCOCCOCCOCCOCCNC(=O)CCC(=O)N2Cc3ccccc3C#Cc3ccccc32)cc1]

### Synthesis of Compound 27 ((S)-2-(2-(2-aminoacetamido)acetamido)-N-(2-((4-(hydroxymethyl)phenyl)amino)-2-oxoethyl)-3-phenylpropanamide)

Compound 22 (0.84 mmol, 1.0 equiv.) was dissolved in MeOH (5 mL), followed by the addition of piperidine (33.6 mmol, 40.0 equiv.). The reaction mixture was stirred at 50° C. for 1 hour. After completion of the reaction, the resulting solution was concentrated under vacuum, and the crude product was purified by column chromatography (DCM/MeOH: 5/1) to yield compound 27 (colorless solid, 95% yield). ¹H NMR (CD₃OD, 400 MHZ): § 3.02 (dd, J=13.9, 9.1 Hz, 1H), 3.24-3.28 (m, 3H), 3.85 (d, J=16.6 Hz, 1H), 3.90 (d, J=12.4 Hz, 1H), 3.94 (d, J=12.2 Hz, 1H), 4.58-4.62 (m, 3H), 7.21-7.34 (m, 7H), 7.64 (d, J=8.5 Hz, 2H).

### Synthesis of Compound 28

Compound 27 (0.88 mmol, 1.0 equiv.), compound 14 (0.97 mmol, 1.1 equiv.) and HATU (1.76 mmol, 2.0 equiv.) were dissolved in DMF (2 mL). The reaction mixture was stirred at room temperature for overnight. After completion of the reaction, the resulting solution was concentrated under vacuum, and the crude product was purified by column chromatography (DCM/MeOH: 8/1) to yield compound 28 (orange oil, 21% yield). ¹H NMR (CD₃OD, 400 MHZ): § 1.97-2.04 (m, 1H), 2.15-2.23 (m, 1H), 2.33-2.40 (m, 1H), 2.54-2.57 (m, 2H), 2.67-2.75 (m, 2H), 3.05 (dd, J=13.8, 9.4 Hz, 1H), 3.24-3.27 (m, 3H), 3.42-3.46 (m, 2H), 3.57-3.65 (m, 25H), 3.69-3.78 (m, 4H), 3.81-3.97 (m, 7H), 4.08 (d, J=16.8 Hz, 1H), 4.58-4.61 (m, 3H), 5.14 (d, J=13.9 Hz, 1H), 7.23-7.37 (m, 13H), 7.46-7.49 (m, 2H), 7.59-7.67 (m, 4H).

### Synthesis of Compound 29 (DBCO-PEG6-GGFG-PAB-CVM-1125)

Compound 28 (0.17 mmol, 1.0 equiv.) and compound 2 (0.26 mmol, 1.5 equiv.) were dissolved in THF (2 mL). The reaction mixture was stirred at 60° C. for 2 hours. After completion of the reaction, the resulting solution was concentrated under vacuum, and the crude product was purified by preparative HPLC to yield compound 29 (white powder, 4% yield). LCMS m/z: [M+H]⁺ Calcd. for C₇₃H₈₀FN₈O₁₈ 1375.56; Found 1375.90.

In this Example, another cleavable linker, vc, was used to replace va linker.

The linker-payload of DBCO-PEG6-vc-PAB-DMEA-CVM-1125 for the present disclosure, compound 33 was prepared by the following Scheme 4.

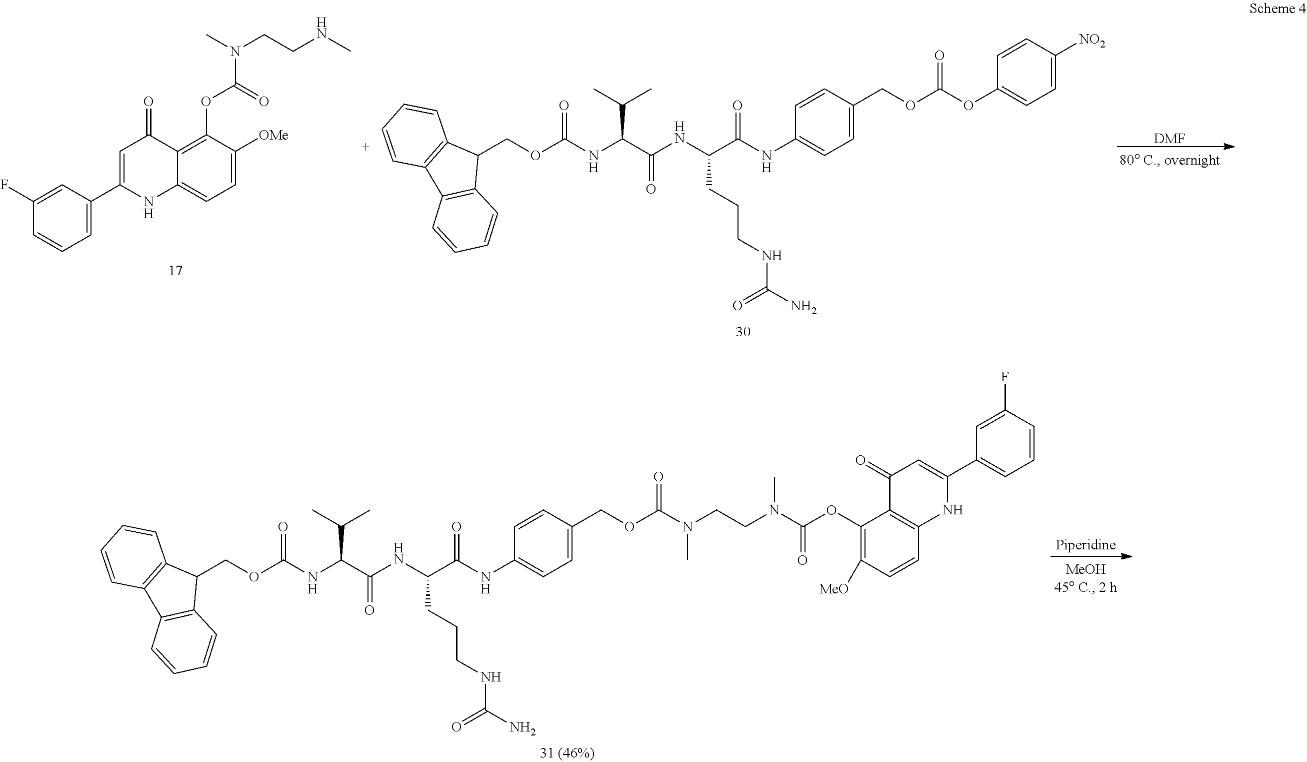

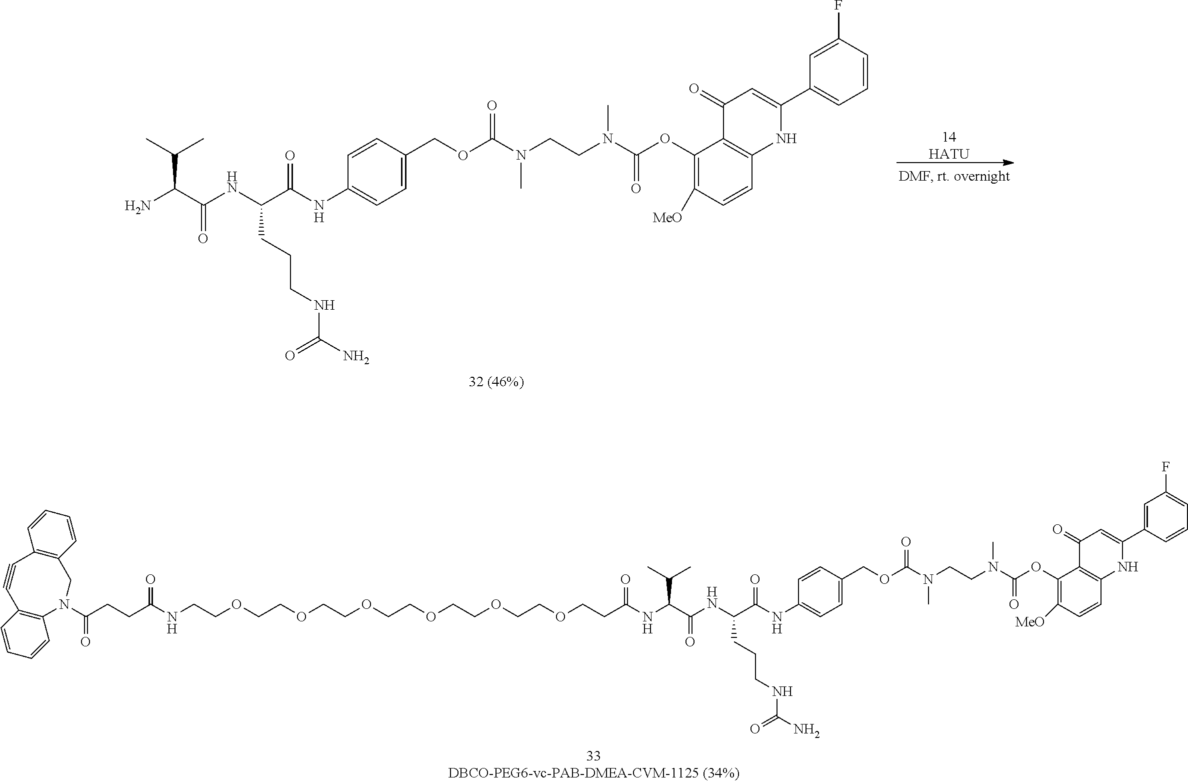 [SMILES: COc1ccc2[nH]c(-c3cccc(F)c3)cc(=O)c2c1OC(=O)N(C)CCN(C)C(=O)OCc1ccc(NC(=O)[C@H](CCCNC(N)=O)NC(=O)[C@@H](N)C(C)C)cc1 ; "14HATU" ; COc1ccc2[nH]c(-c3cccc(F)c3)cc(=O)c2c1OC(=O)N(C)CCN(C)C(=O)OCc1ccc(NC(=O)[C@H](CCCNC(N)=O)NC(=O)[C@@H](NC(=O)CCOCCOCCOCCOCCOCCOCCNC(=O)CCC(=O)N2Cc3ccccc3C#Cc3ccccc32)C(C)C)cc1]

### Synthesis of Compound 31 (4-((S)-2-((S)-2-((((9H-fluoren-9-yl)methoxy)carbonyl)amino)-3-methylbutanamido)-5-ureidopentanamido)benzyl(2-(3-fluorophenyl)-6-methoxy-4-oxo-1,4-dihydroquinolin-5-yl)ethane-1,2-diylbis(methylcarbamate))

Compound 17 (1.66 mmol, 2.5 equiv.) and compound 30 (0.67 mmol, 1.0 equiv.) were dissolved in DMF (20 mL). The reaction mixture was stirred at 60° C. for overnight. After completion of the reaction, the resulting solution was concentrated under vacuum, and the crude product was purified by column chromatography (DCM/MeOH: 50/1 to 5/1) to yield compound 31 (yellow powder, 46% yield). 1H NMR (CD₃OD, 400 MHZ): § 0.90-1.56 (br, 6H), 1.45-1.60 (br, 2H), 1.68-1.83 (br, 1H), 1.84-1.97 (br, 1H), 2.03-2.20 (br, 1H), 2.91-3.28 (m, 8H), 3.56-3.93 (m, 7H), 3.95-4.08 (m, 1H), 4.31-4.45 (m, 2H), 4.49-4.61 (m, 1H), 5.50-5.21 (m, 2H), 6.27-6.40 (br, 1H), 7.14-7.44 (m, 8H), 7.45-7.72 (m, 8H), 7.73-7.83 (m, 2H).

### Synthesis of Compound 32 (4-((S)-2-((S)-2-amino-3-methylbutanamido)-5-ureidopentanamido)benzyl(2-(3-fluorophenyl)-6-methoxy-4-oxo-1,4-dihydroquinolin-5-yl)ethane-1,2-diylbis(methylcarbamate))

Compound 31 (0.31 mmol, 1.0 equiv.) was dissolved in MeOH (18 mL), followed by the addition of piperidine (1.8 mL). The reaction mixture was stirred at 45° C. for 2 hours. After completion of the reaction, the resulting solution was concentrated under vacuum, and the crude product was purified by column chromatography (DCM/MeOH: 50/1 to 10/1) to yield compound 32 (yellow powder, 46% yield). ¹H NMR (CD₃OD, 400 MHZ): δ 0.89-1.45 (m, 6H), 1.51-1.62 (br, 2H), 1.69-1.82 (br, 2H), 1.82-1.95 (br, 1H), 1.96-2.10 (br, 1H), 2.92-3.32 (m, 10H), 3.61-3.89 (m, 5H), 3.96-4.23 (m, 1H), 4.45-4.60 (m, 1H), 5.02-5.20 (m, 2H), 6.35 (d, J=12.9 Hz, 1H), 7.20-7.47 (m, 4H), 7.47-7.68 (m, 6H).

### Synthesis of Compound 33 (DBCO-PEG6-vc-PAB-DMEA-CVM-1125)

Compound 32 (0.13 mmol, 1.5 equiv.), compound 14 (0.09 mmol, 1.0 equiv.) and HATU (0.26 mmol, 3.0 equiv.) were dissolved in DMF (2 mL). The reaction mixture was stirred at room temperature for overnight. After completion of the reaction, the resulting solution was concentrated under vacuum, and the crude product was purified by column chromatography (DCM/MeOH: 50/1 to 5/1) to yield compound 33 (brown oil, 34% yield). ¹H NMR (CD₃OD, 400 MHZ): § 0.94-1.05 (m, 6H), 1.49-1.68 (br, 2H), 1.68-1.82 (br, 1H), 1.83-2.07 (m, 2H), 2.07-2.23 (m, 2H), 2.30-2.41 (m, 1H), 2.52-2.62 (m, 2H), 2.64-2.75 (m, 1H), 2.85-3.15 (m, 7H), 3.16-3.29 (m, 3H), 3.39-3.51 (m, 3H), 3.52-3.63 (m, 20H), 3.66-3.90 (m, 7H), 3.91-4.10 (m, 1H), 4.12-4.29 (m, 1H), 4.41-4.55 (m, 1H), 4.55-4.67 (m, 1H), 4.96-5.21 (m, 3H), 6.23-6.38 (m, 1H), 7.07-7.49 (m, 10H), 7.50-7.88 (m, 8H).

In this Example, the linker-payload of MC-vc-PAB-DMEA-CVM-1125 for the present disclosure, compound 35 was prepared by the following Scheme 5.

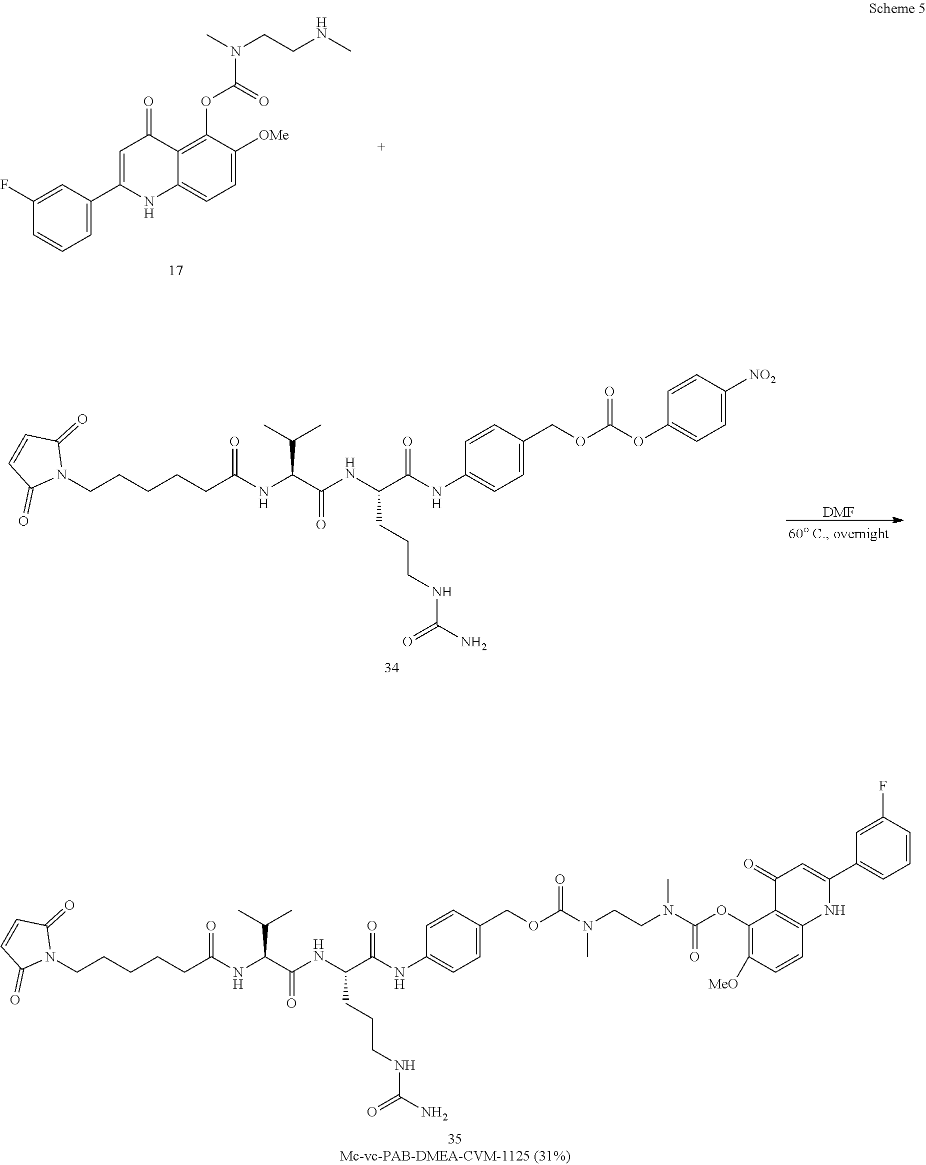

### Synthesis of Compound 35 (MC-vc-PAB-DMEA-CVM-1125)

Compound 17 (0.16 mmol, 2.5 equiv.) and compound 34 (0.06 mmol, 1.0 equiv.) were dissolved in DMF (2 mL). The reaction mixture was stirred at 60° C. for overnight. After completion of the reaction, the resulting solution was concentrated under vacuum, and the crude product was purified by column chromatography (DCM/MeOH: 50/1 to 5/1) to yield compound 35 (brown oil, 31% yield). ¹H NMR (CD₃OD, 400 MHZ): δ 0.94-1.07 (m, 6H), 1.26-1.38 (m, 4H), 1.51-1.70 (m, 6H), 1.71-1.83 (m, 1H), 1.85-2.01 (m, 1H), 2.03-2.18 (m, 2H), 2.91-3.29 (m, 8H), 3.40-3.54 (m, 3H), 3.70-3.94 (m, 4H), 4.16-4.26 (m, 1H), 5.00-5.26 (m, 2H), 6.35 (s, 1H), 6.81 (d, J=4.6 Hz, 2H), 7.19-7.74 (m, 10H).

In this Example, branched PEG was used to make the linking group (L) be able to connect more drugs. The linker-payload thus obtained was named compound 38, which has a structure of DBCO-PEG6-branched-(PEG4-va-PAB-DMEA-CVM-1125) 2 in the following Scheme 6.

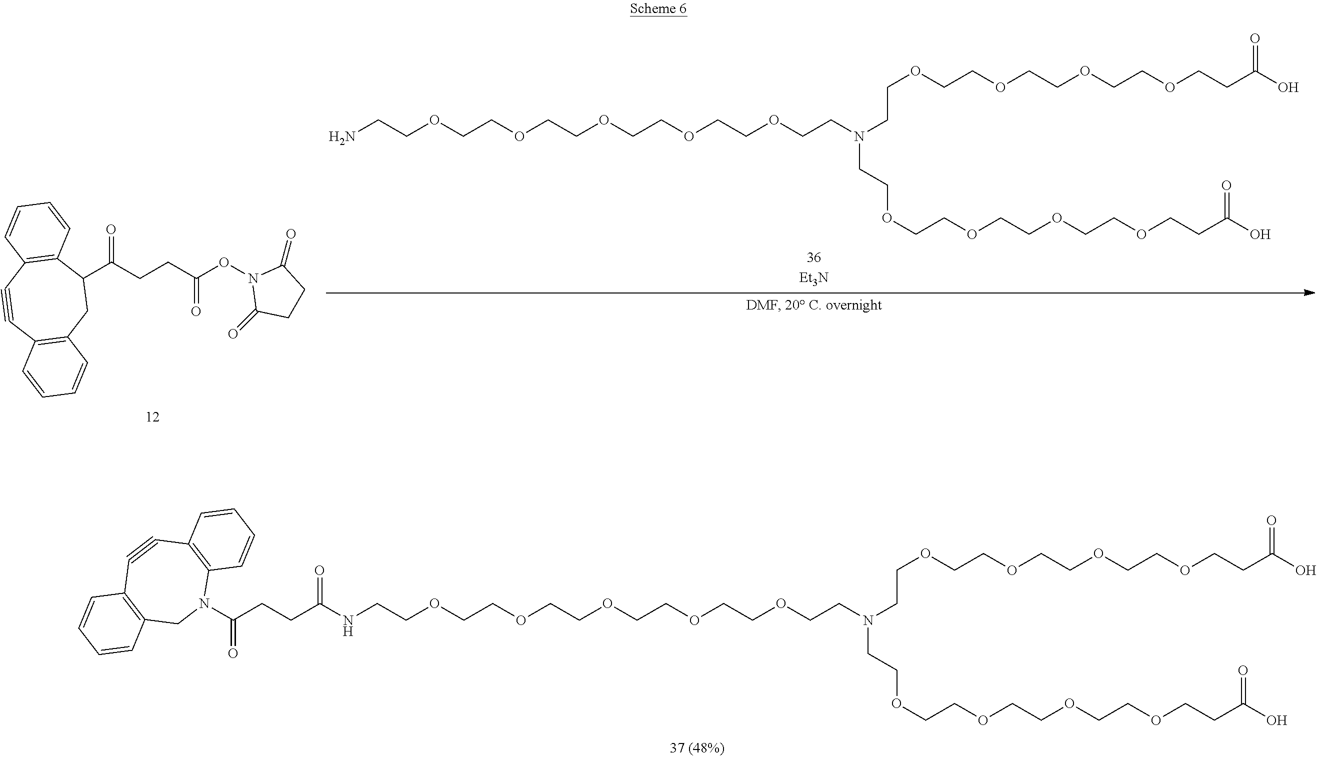 [SMILES: O=C(CCC(=O)C1Cc2ccccc2C#Cc2ccccc21)ON1C(=O)CCC1=O ; NCCOCCOCCOCCOCCOCCN(CCOCCOCCOCCOCCC(=O)O)CCOCCOCCOCCOCCC(=O)O ; O=C(O)CCOCCOCCOCCOCCN(CCOCCOCCOCCOCCOCCNC(=O)CCC(=O)N1Cc2ccccc2C#Cc2ccccc21)CCOCCOCCOCCOCCC(=O)O]

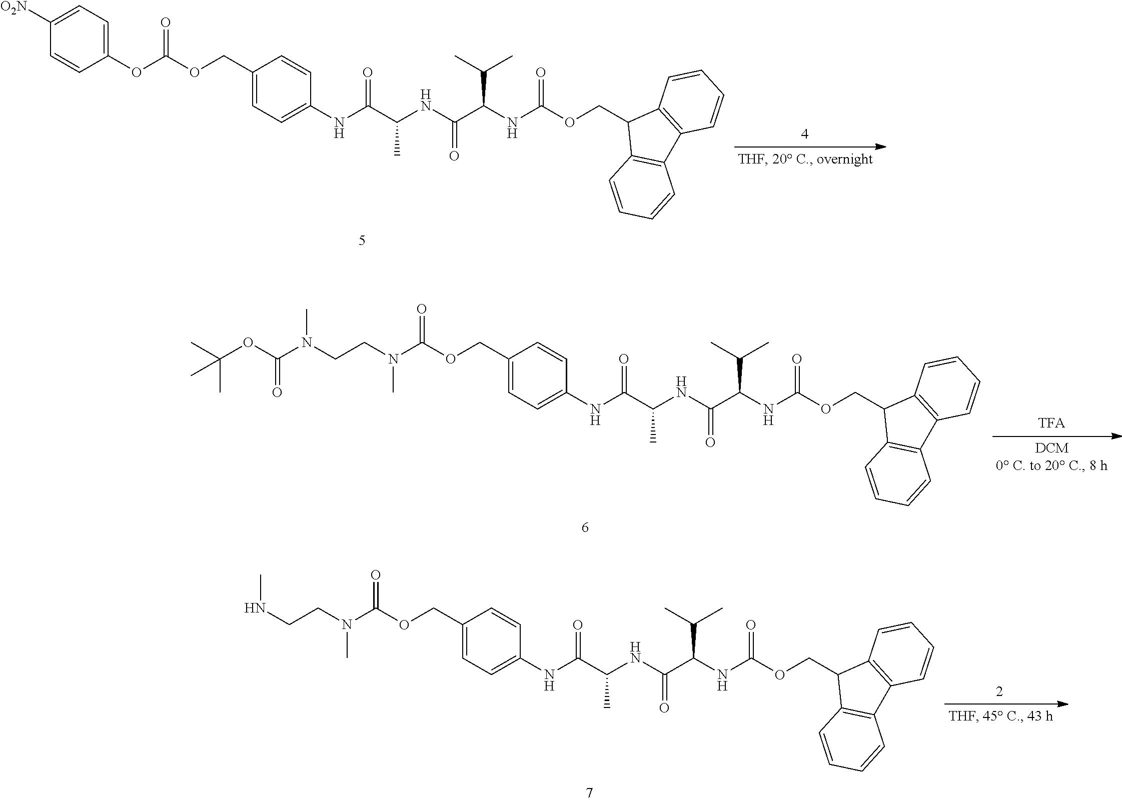 [SMILES: CC(C)[C@@H](NC(=O)OCC1c2ccccc2-c2ccccc21)C(=O)N[C@H](C)C(=O)Nc1ccc(COC(=O)Oc2ccc([N+](=O)[O-])cc2)cc1 ; CC(C)[C@@H](NC(=O)OCC1c2ccccc2-c2ccccc21)C(=O)N[C@H](C)C(=O)Nc1ccc(COC(=O)N(C)CCN(C)C(=O)OC(C)(C)C)cc1 ; CNCCN(C)C(=O)OCc1ccc(NC(=O)[C@@H](C)NC(=O)[C@H](NC(=O)OCC2c3ccccc3-c3ccccc32)C(C)C)cc1]

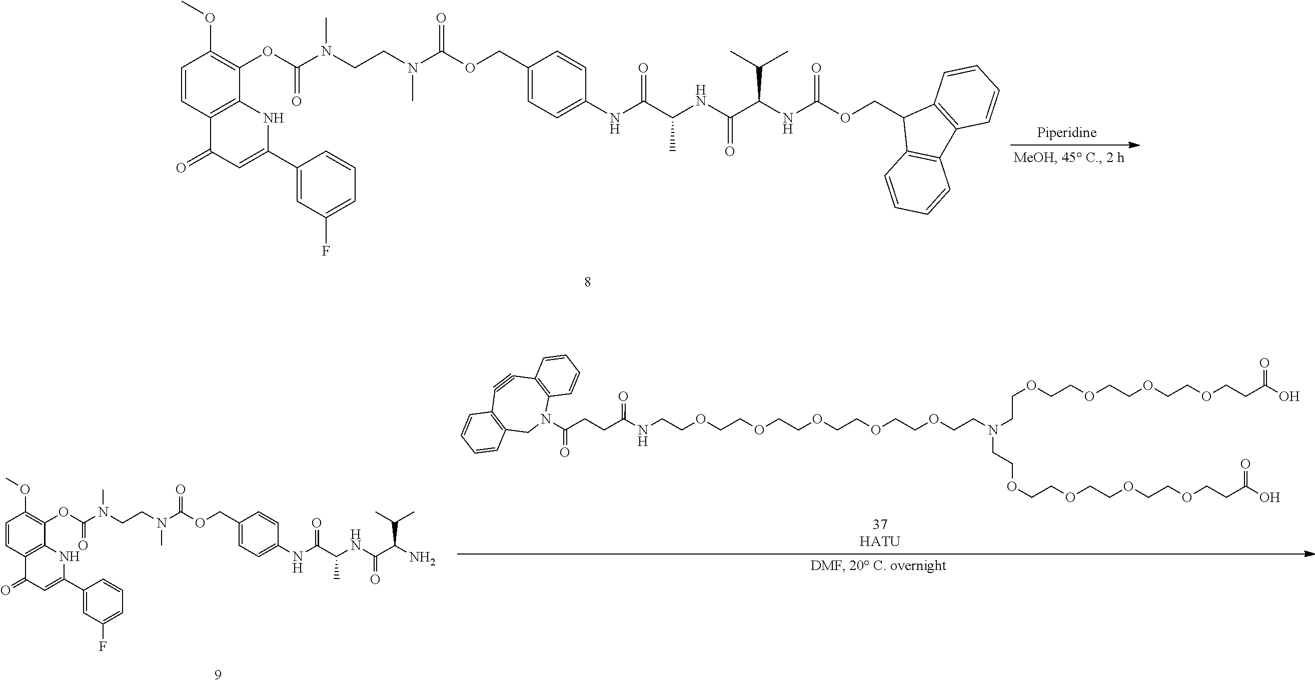 [SMILES: COc1ccc2c(=O)cc(-c3cccc(F)c3)[nH]c2c1OC(=O)N(C)CCN(C)C(=O)OCc1ccc(NC(=O)[C@@H](C)NC(=O)[C@H](NC(=O)OCC2c3ccccc3-c3ccccc32)C(C)C)cc1 ; COc1ccc2c(=O)cc(-c3cccc(F)c3)[nH]c2c1OC(=O)N(C)CCN(C)C(=O)OCc1ccc(NC(=O)[C@@H](C)NC(=O)[C@H](N)C(C)C)cc1 ; O=C(O)CCOCCOCCOCCOCCN(CCOCCOCCOCCOCCOCCNC(=O)CCC(=O)N1Cc2ccccc2C#Cc2ccccc21)CCOCCOCCOCCOCCC(=O)O]

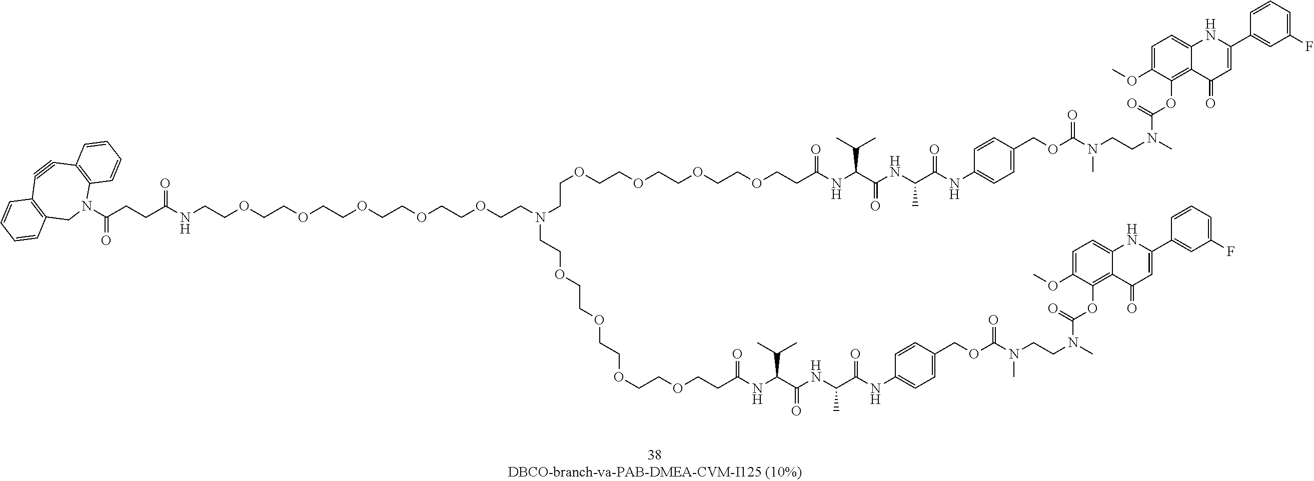 [SMILES: COc1ccc2[nH]c(-c3cccc(F)c3)cc(=O)c2c1OC(=O)N(C)CCN(C)C(=O)OCc1ccc(NC(=O)[C@H](C)NC(=O)[C@@H](NC(=O)CCOCCOCCOCCOCCN(CCOCCOCCOCCOCCOCCNC(=O)CCC(=O)N2Cc3ccccc3C#Cc3ccccc32)CCOCCOCCOCCOCCC(=O)N[C@H](C(=O)N[C@@H](C)C(=O)Nc2ccc(COC(=O)N(C)CCN(C)C(=O)Oc3c(OC)ccc4[nH]c(-c5cccc(F)c5)cc(=O)c34)cc2)C(C)C)C(C)C)cc1]

### Synthesis of Compound 37

Compound 12 (0.07 mmol, 1.0 equiv.) was dissolved in DMF (0.5 mL), followed by the addition of compound 36 (0.08 mmol, 1.1 equiv.) and Et₃N (0.35 mmol, 5 equiv.). The reaction mixture was stirred at room temperature overnight. After completion of the reaction, the resulting solution was concentrated under vacuum, and the crude product was purified by column chromatography (pure MeOH) to yield compound 37 (brown oil, 48% yield). ¹H NMR (CDCl₃, 400 MHZ): § 1.94 (dt, J=16.8, 6.3 Hz, 1H), 2.17 (dt, J=15.5, 6.3 Hz, 1H), 2.44-2.51 (m, 5H), 5.75-2.81 (m, 7H), 3.31-3.35 (m, 2H), 3.41-3.50 (m, 3H), 3.54-3.71 (m, 64H), 5.15 (d, J=13.8 Hz, 1H), 6.46 (br, 1H), 7.24-7.41 (m, 6H), 7.52-7.54 (m, 1H), 7.67 (d, J=7.4 Hz, 1H).

### Synthesis of Compound 38 (DBCO-PEG6-branched-(PEG4-va-PAB-DMEA-CVM-1125) 2)

Compound 37 (0.032 mmol, 1 equiv.) and compound 9 (0.065 mmol, 2.0 equiv.) were dissolved in DMF (1.0 mL), followed by the addition of HATU (0.13 mmol, 4.0 equiv.). The mixture was stirred at room temperature overnight, then concentrated and dried. The crude product was purified by column chromatography (DCM/MeOH: 6/1) to yield compound 38 (brown solid, 10% yield). ¹H NMR (CD₃OD, 400 MHZ): & 0.98-1.02 (m, 12H), 1.43-1.45 (m, 6H), 1.94-2.01 (m, 1H), 2.14-2.21 (m, 3H), 2.33-2.14 (m, 1H), 2.56 (br, 4H), 2.67-2.75 (m, 1H), 2.95-3.12 (m, 16H), 3.23-3.26 (m, 3H), 3.41-3.46 (m, 3H), 3.56-3.64 (m, 47H), 3.71-3.89 (m, 15H), 4.22-4.26 (m, 1H), 4.48-4.51 (m, 1H), 5.02-5.15 (m, 7H), 6.35 (br, 2H), 7.25-7.26 (m, 2H), 7.32-7.42 (m, 8H), 7.45-7.66 (m, 17H).

### Comparative Example 1: Different Activation Reagent to Activate CVM-1125 (compound 1)

In this Example, phenyl chloroformate and Boc₂O/DMAP were respectively used to replace the activation reagent used in the Example 1, i.e., 4-nitrobenzyl chloroformate.

The activation reagent, reaction conditions and result are listed in Table 4 and Scheme 8.

TABLE 4

| | Activation | | | | | |
|---|---|---|---|---|---|---|
| Entry | Reagent | Solvent | Time | Temperature | Base | Result |
| 1 | Phenyl | DMF | 8 | Room temperature | NaH | CVM-1125 |
| | chloroformate | | hours | | | |
| 2 | Phenyl | THF | 24 | Room temperature | NaH | Decompose |
| | chloroformate | | hours | | | |
| 3 | Phenyl | THF | 24 | 60° C. | NaH | Decompose |
| | chloroformate | | hours | | | |
| 4 | Phenyl | CH₃CN | 20 | 60° C. | K₂CO₃ | CVM-1125 |
| | chloroformate | | hours | | | |
| 5 | Phenyl | CH₂Cl₂ | 50 | Room temperature | Et₃N | CVM-1125 |
| | chloroformate | | hours | to 40° C. | | |
| 6 | Boc₂O/DMAP | THF | 9 | Room temperature | — | CVM-1125 |
| | | | hours | | | |

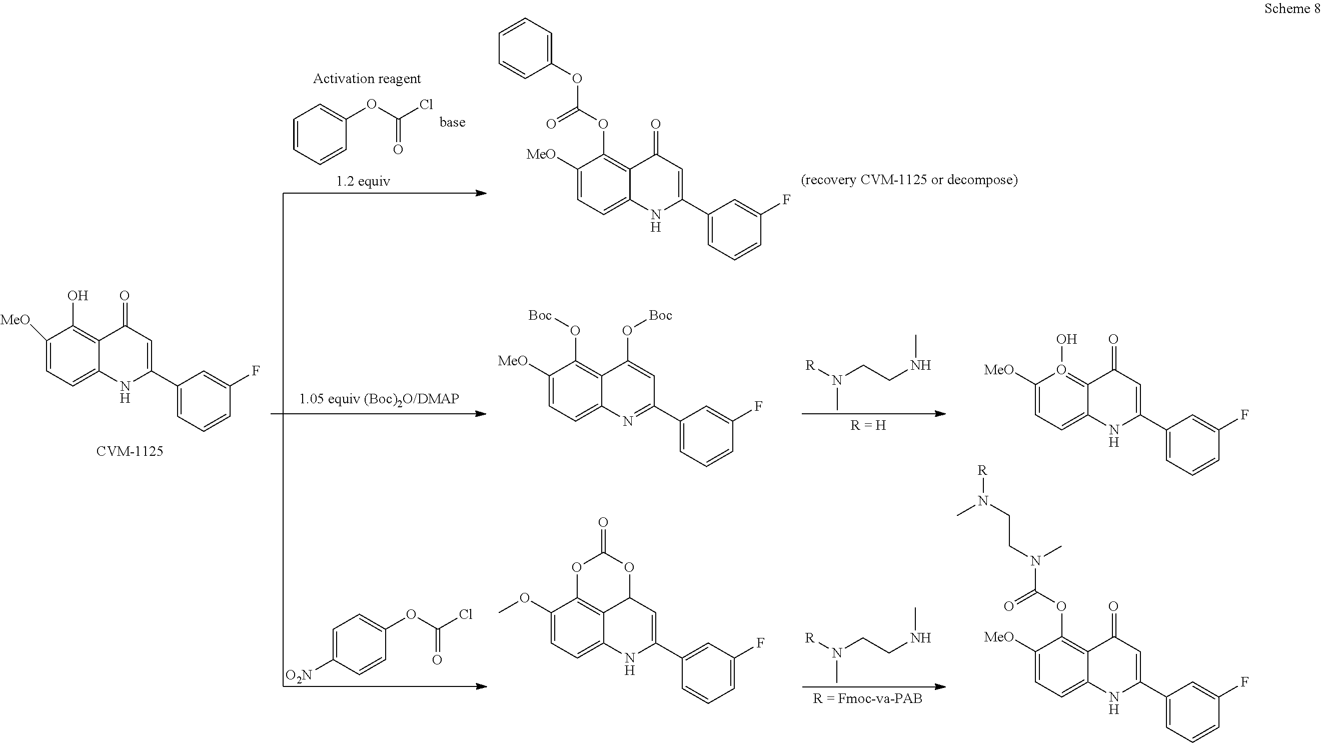

In Table 4, phenyl chloroformate or di-tert-butyl dicarbonate (Boc₂O) cannot successfully activate the phenol group on the CVM-1125. Even trying to change the reaction temperature, reaction time, reaction solvent, and the use of different bases, the product thus obtained was either recovered to the starting material or decomposed.

The above result shows that the synthesis of the linking group to the OH group of CVM-1125 (compound 1) proved challenging due to the instability of the phenyl carbonate activation intermediate. This intermediate exhibited high susceptibility to decomposition or reconversion back into CVM-1125 under various reaction conditions.

### Example 2: The Preparation of Antibody-Drug Conjugation for the Present Disclosure

The antibody-drug conjugation of CVM-ADC01 for the present disclosure can be prepared by the following Scheme 9.

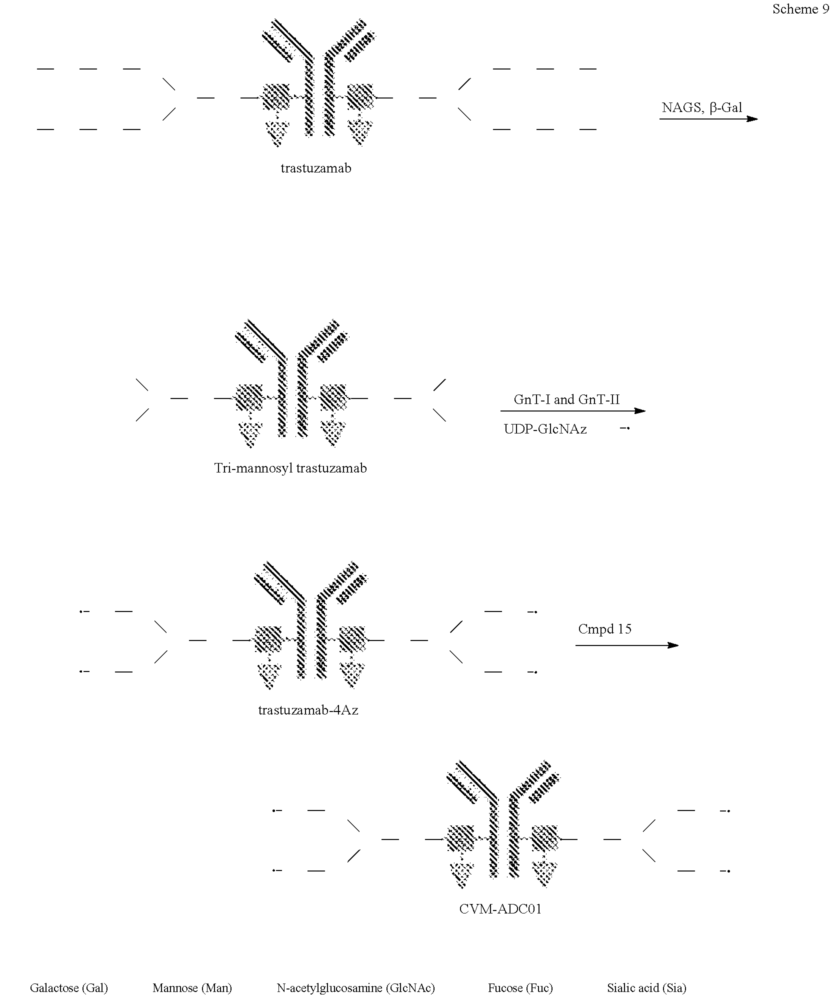

### Preparation of Trimannosyl-Trastuzumab:

Trastuzumab (100 mg) in 100 mM citrate buffer pH 6.08 was incubated with 2.0 mL of β-Galactosidase (12.5 units/μL) and 0.8 mL β-N-Acetylglucosaminidase S (8.8 unit/μL) at 37° C. for 3 hours. The digested antibody sample was analyzed by reduced LC-MS without further purification before proceeding to the next enzyme reaction step.

### Preparation of Trastuzumab-4Az:

Trastuzumab-Trimannosyl (100 mg) was loaded onto a 5 mL Protein A resin (Hitrap Protein A HP) column. The column was washed with 1 column volume (CV) of 1× phosphate-buffered saline (PBS) followed by 15 CVs of 25 mM MOPS pH 7.5 buffer. Subsequently, a mixture containing UDP-GlcNAz (50 mg), GnT-I (6.67 mg), and GnT-II (1.43 mg) in a buffer solution (25 mM MOPS pH 7.52 with 100 mM MnCl2) was passed through the Protein A resin column at room temperature for 3 hours. Finally, the elution of the trastuzumab-4Az product was achieved using 0.1 M sodium citrate pH 3.0 and neutralized with 3 M Tris-base pH 9.0. The protein solution was then collected and concentrated, yielding 96.84 mg of trastuzumab-4Az with a concentration of 4.53 mg/mL.

### Conjugation of CVM-ADC01 (Trastuzumab-Linking Group-4CVM-1125):

To a solution of trastuzumab-4Az (75 mg, 4.53 mg/mL) in buffer (0.1 M Na-citrate, 0.4 M Tris-HCl, pH 6.3), a mixture of DMSO (2.6 mL) and compound 15 (i.e., DBCO-PEG6-va-PAB-DMEA-CVM-1125)(10 mM stock solution in DMSO, 14 equivalents relative to antibody, 0.71 mL) was slowly added. The reaction mixture was incubated in a shaking incubator (150 rpm) at 37° C. for 20 hours. Concentration of CVM-ADC01 and removal of unconjugated linker-payload were performed using an Amicon Ultra-15 centrifugal filter with a 30 kDa NMWL and 25 mM Na-citrate pH 6.5 (settings: 4° C., 4000×g, 20 minutes; repeated three times) to afford 92.1 mg CVM-ADC01 (10.466 mg/mL) with >99% recovery yield. The concentration of CVM-ADC01 was estimated by measuring absorbance at 280 nm (using an absorption coefficient at 280 nm of 1.45 mL mg⁻¹ cm⁻¹), and the A280 value was used for calculation of recovery yield. The drug-to-antibody ratio (DAR) of the ADC is 3.60 which was determined by LC-MS. The SEC and SDS-PAGE analysis of CVM-ADC01 showed that the high purity and no aggregation observed.

The structure of CVM-ADC01 is shown below:

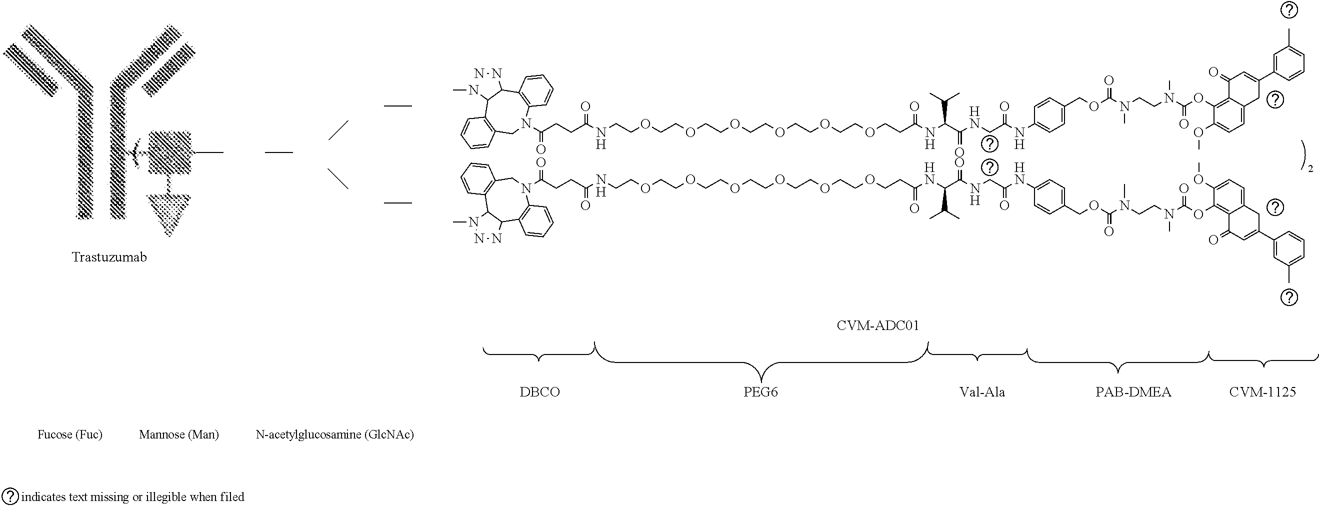

The antibody-drug conjugation of CVM-ADC02 for the present disclosure can be prepared by the following Scheme 10.

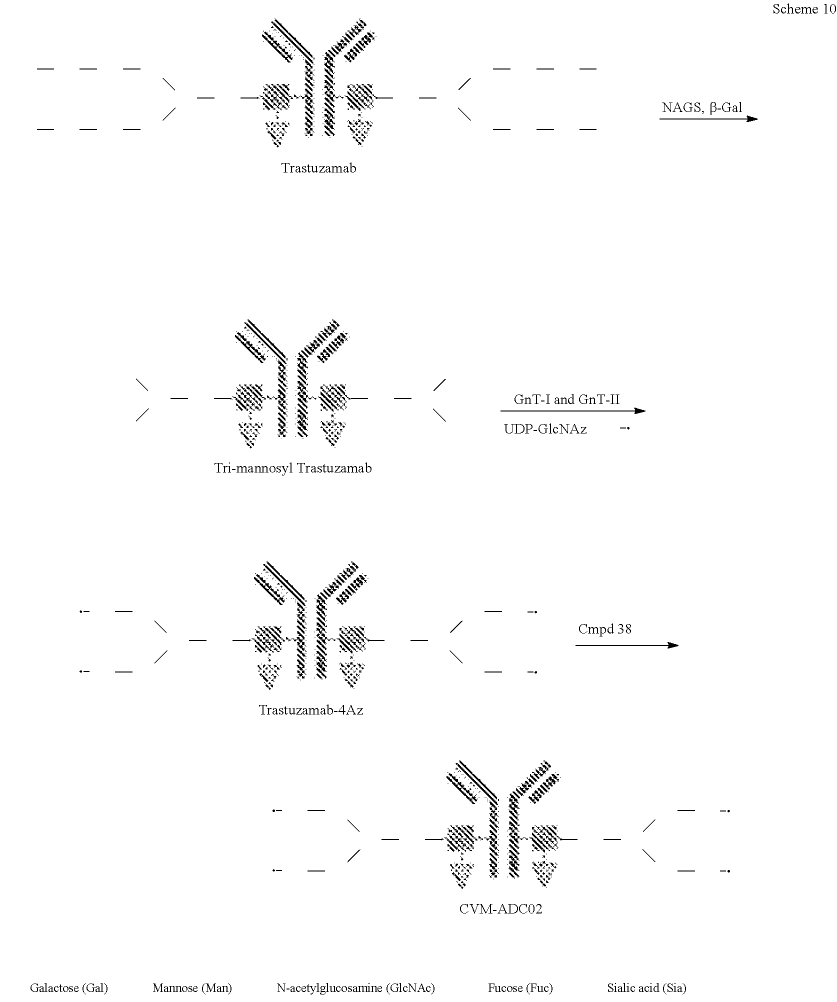

### Preparation of ADC for CVM-ADC02

a) Take 2.900 mL of antibody (5.173 mg/mL, 15 mg) into a new 50 mL tube.
b) Use 0.100 mL 25 mM MOPS pH7.5 to adjust the final antibody reaction concentration to 5 mg/mL.
c) Add 0.700 mL of DMA to make up 30% of the total volume containing DMA.
d) Add 0.200 mL of compound 38 (i.e., DBCO-PEG6-branched-(PEG4-va-PAB-DMEA-CVM-1125) 2)(10 mM in DMA, 20 equivalents) to the 50 mL tube.
e) Incubate the solution at 37° C. and shake for 18 hours.

### Purification of ADC for CVM-ADC02

a) Use a 30 kDa centrifugal filter to replace the solution with 25 mM MOPS pH7.5 buffer (centrifuge at 4000×g, 4° C. for 30 minutes) and repeat this step 3 times.
b) Filter the solution through a 0.22 μm filter (Yield: >99% and DAR: 7.86).

The structure of CVM-ADC02 is shown below:

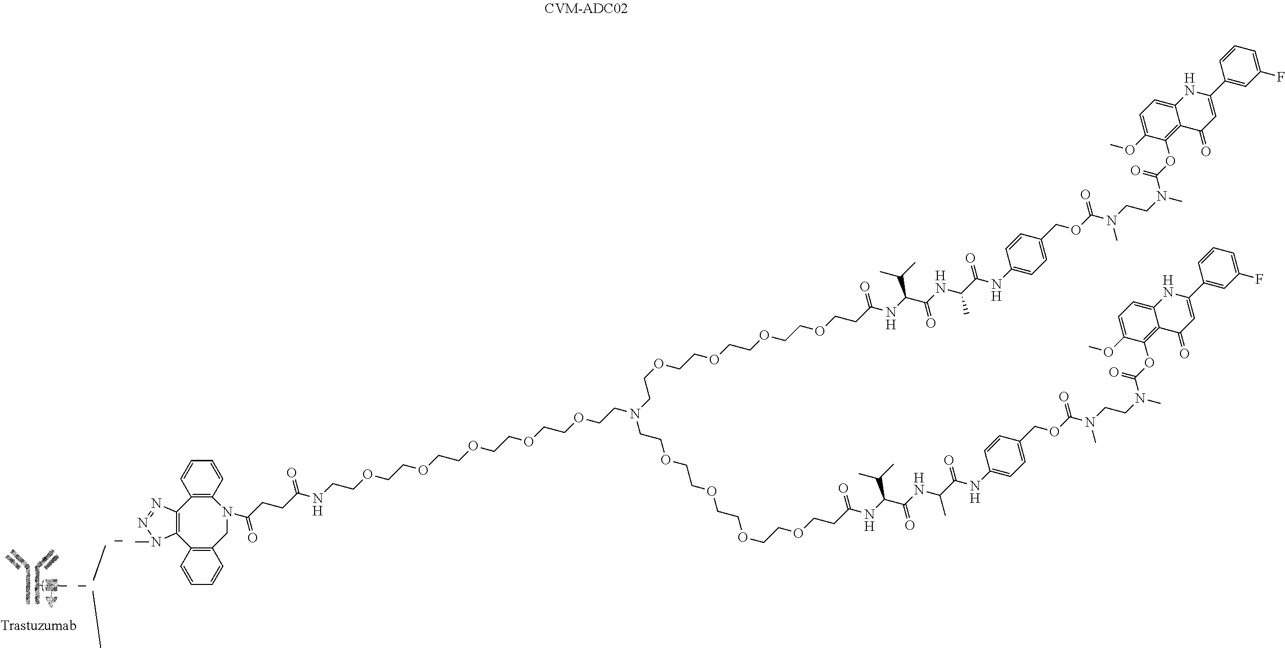 [SMILES: COc1ccc2[nH]c(-c3cccc(F)c3)cc(=O)c2c1OC(=O)N(C)CCN(C)C(=O)OCc1ccc(NC(=O)C(C)NC(=O)[C@@H](NC(=O)CCOCCOCCOCCOCCN(CCOCCOCCOCCOCCOCCNC(=O)CCC(=O)N2Cc3ccccc3-c3c(nnn3C)-c3ccccc32)CCOCCOCCOCCOCCC(=O)N[C@H](C(=O)N[C@@H](C)C(=O)Nc2ccc(COC(=O)N(C)CCN(C)C(=O)Oc3c(OC)ccc4[nH]c(-c5cccc(F)c5)cc(=O)c34)cc2)C(C)C)C(C)C)cc1]

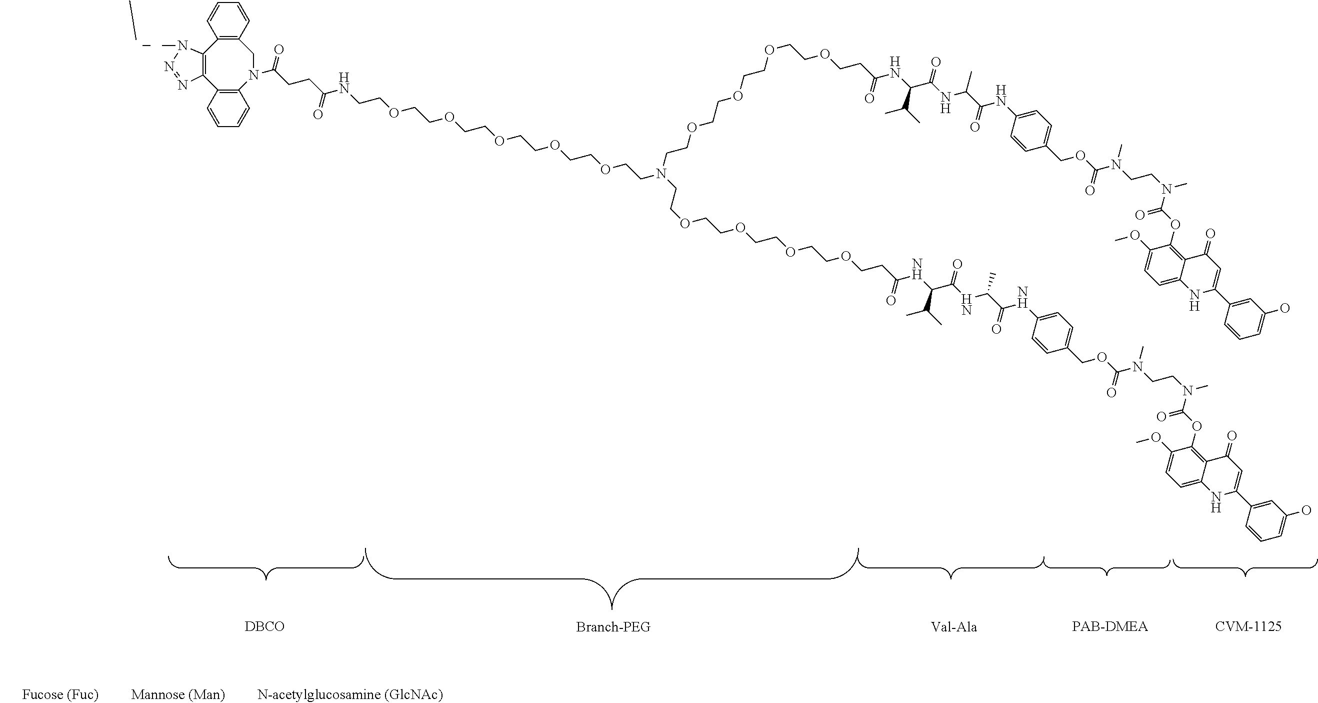

The antibody-drug conjugation of CVM-ADC10 for the present disclosure can be prepared by the following Scheme 11.

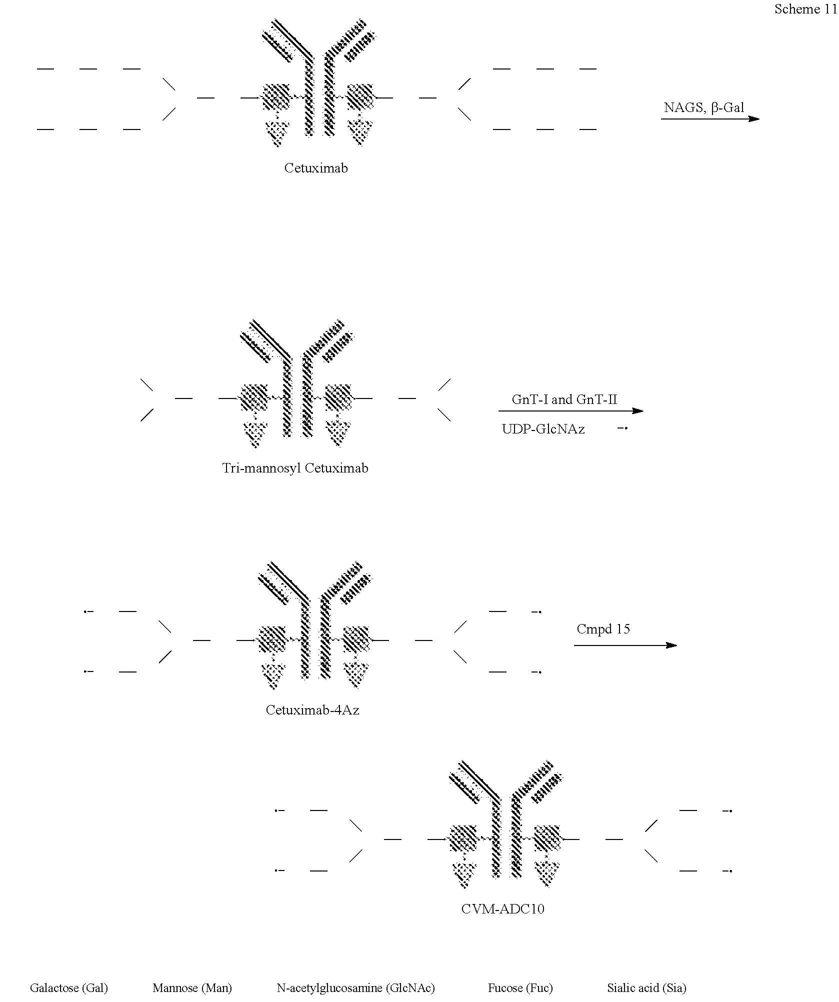

### Preparation of ADC for CVM-ADC10

a) Take 4.607 mL of antibody (4.341 mg/mL, 20 mg) into a new 50 mL tube.
b) Use 0.044 mL ddH₂O to adjust the final antibody reaction concentration to 4.3 mg/mL.
c) Add 0.664 mL of DMSO to make up 20% of the total volume containing DMSO.
d) Add 0.267 mL of compound 15 (i.e., DBCO-PEG6-va-PAB-DMEA-CVM-1125)(10 mM in DMSO, 20 equivalents) to the 50 mL tube.
e) Incubate the solution at 37° C. and shake for 18 hours.

### Purification of ADC for CVM-ADC10

a) Use a 30 kDa centrifugal filter to replace the solution with 25 mM Na-citrate pH 6.5 buffer (centrifuge at 4000×g, 4° C. for 30 minutes) and repeat this step 3 times.
b) Filter the solution through a 0.22 μm filter (Yield: >99% and DAR: 3.41).

The structure of CVM-ADC10 is shown below:

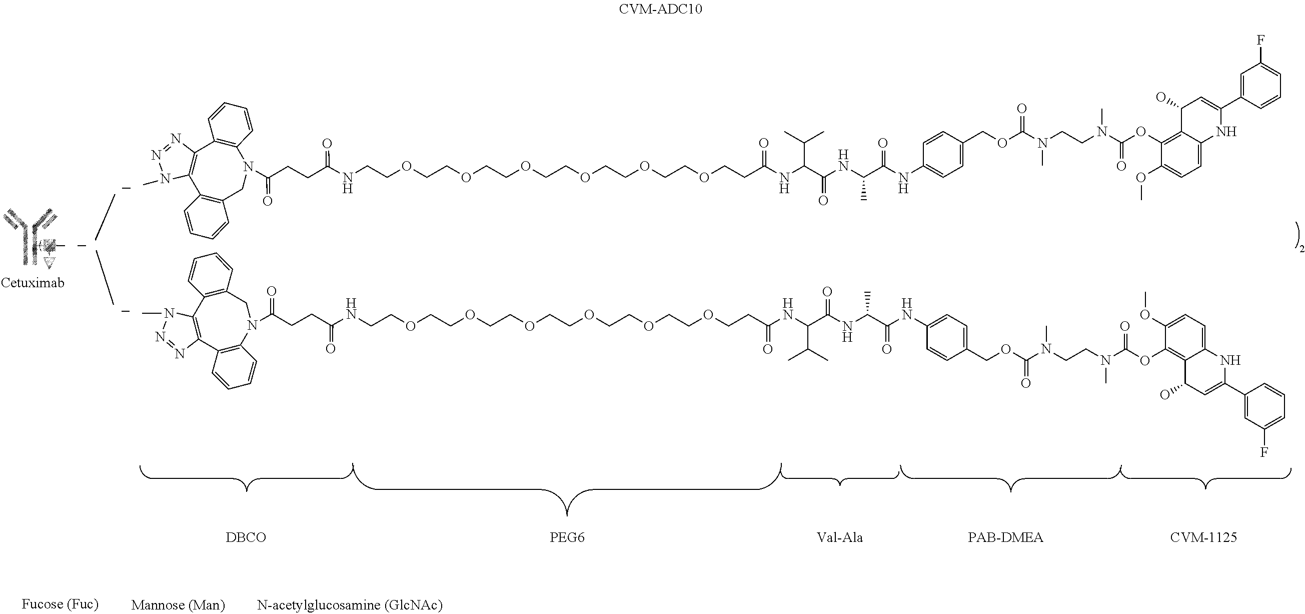

The antibody-drug conjugation of CVM-ADC14 for the present disclosure can be prepared by the following Scheme 12.

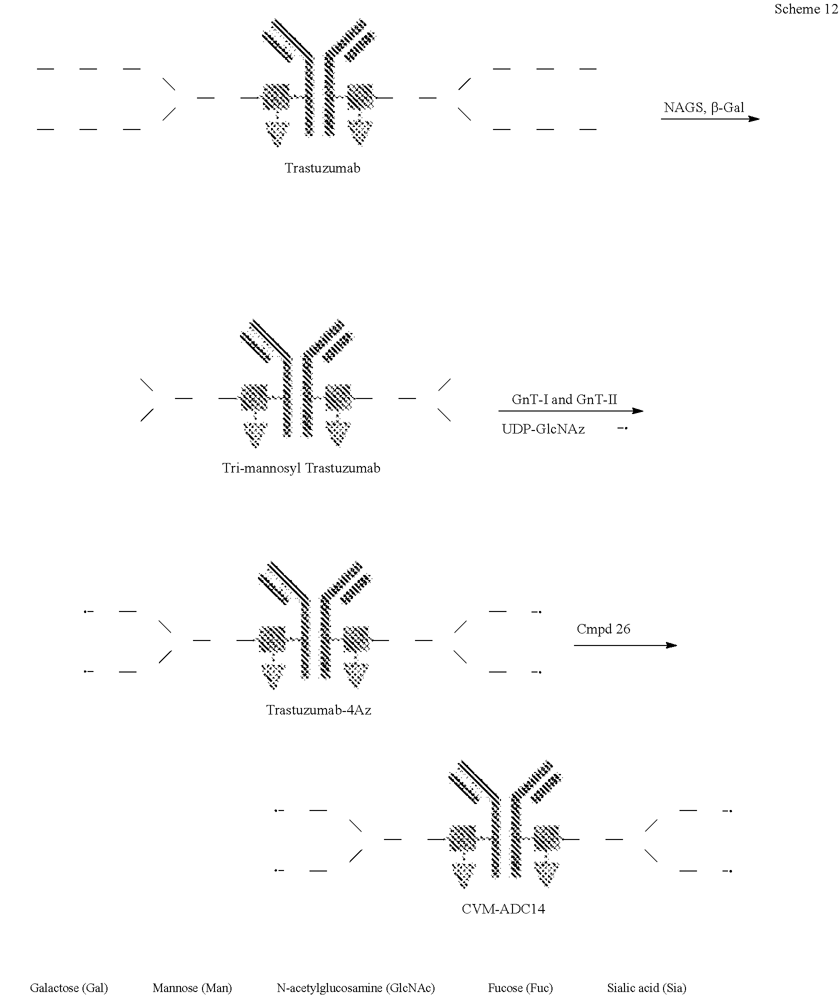

### Preparation of ADC for CVM-ADC14

a) Take 4.79 mL of antibody (6.264 mg/mL, 30 mg) into a new 50 mL tube.
b) Use 1.21 mL ddH₂O to adjust the final antibody reaction concentration to 5 mg/mL.
c) Add 0.8 mL of DMA to make up 20% of the total volume containing DMA.
d) Add 0.533 mL of compound 26 (i.e., DBCO-PEG6-GGFG-PAB-DMEA-CVM-1125)(10 mM in DMA, 20 equivalents) to the 50 mL tube.
e) Incubate the solution at 37° C. and shake for 18 hours.

### Purification of ADC for CVM-ADC14

a) Use a 30 kDa centrifugal filter to replace the solution with 20 mM pH 6.0 Histidine (centrifuge at 4800 rpm, 4° C. for 30 minutes) and repeat this step 4 times.
b) Filter the solution through a 0.22 μm filter (Yield: 135% and DAR: 3.56).

The structure of CVM-ADC14 is shown below:

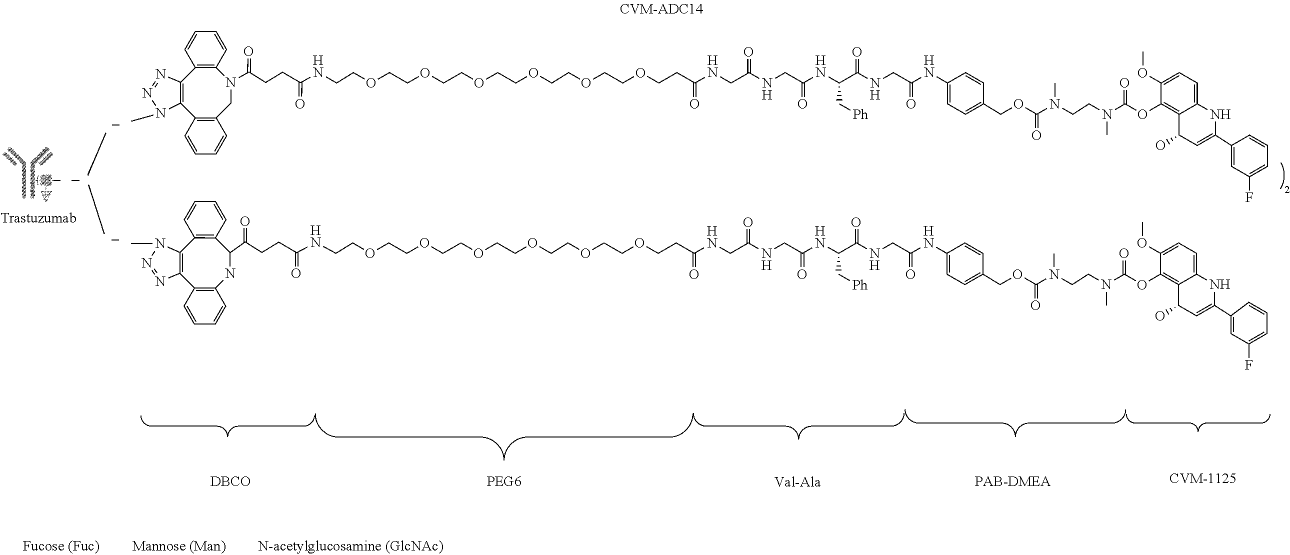

The antibody-drug conjugation of CVM-ADC17 for the present disclosure can be prepared by the following Scheme 13.

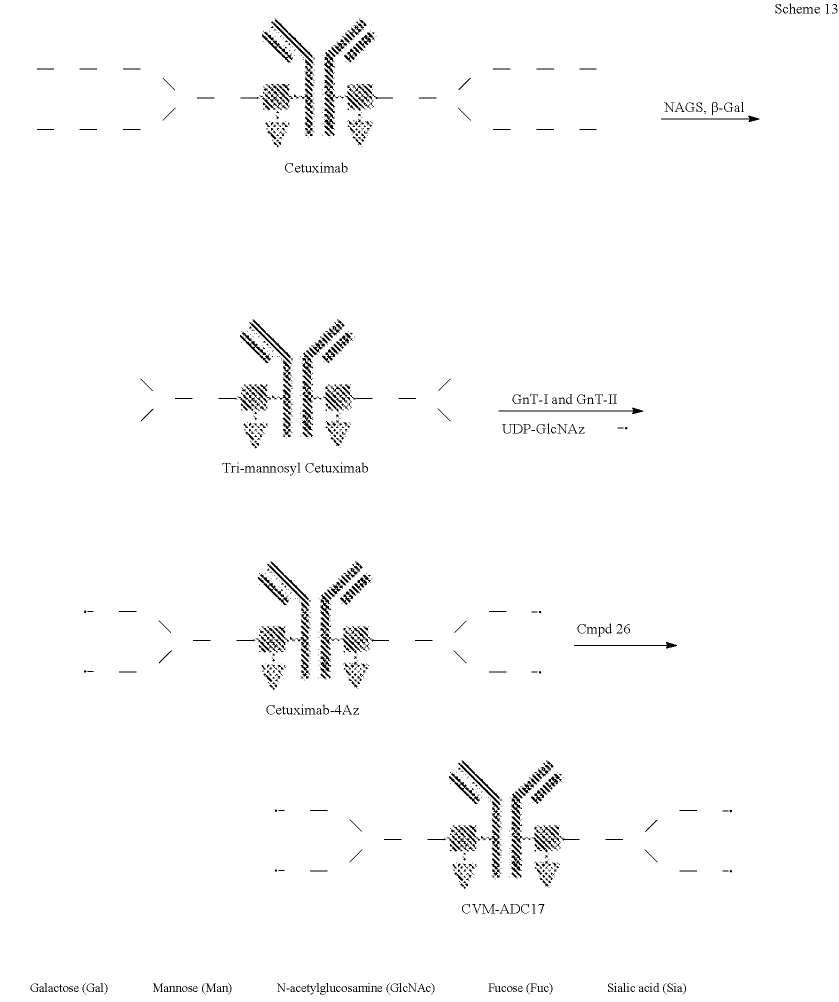

### Preparation of ADC for CVM-ADC17

a) Take 3.369 mL of antibody (6.53 mg/mL, 22 mg) into a new 50 mL tube.
b) Use 1.031 mL 25 mM MOPS pH 7.5 to adjust the final antibody reaction concentration to 5.0 mg/mL.
c) Add 0.587 mL of DMA to make up 20% of the total volume containing DMA.
d) Add 0.293 mL of compound 26 (i.e., DBCO-PEG6-GGFG-PAB-DMEA-CVM-1125)(10 mM in DMA, 20 equivalents) to the 50 mL tube.
e) Incubate the solution at 37° C. and shake for 18 hours.

### Purification of ADC for CVM-ADC17

a) Use a 30 kDa centrifugal filter to replace the solution with 25 mM MOPS pH 7.5 buffer (centrifuge at 4000×g, 4° C. for 30 minutes) and repeat this step 3 times.
b) Filter the solution through a 0.22 μm filter (Yield: 69% and DAR: 3.8).

The structure of CVM-ADC17 is shown below:

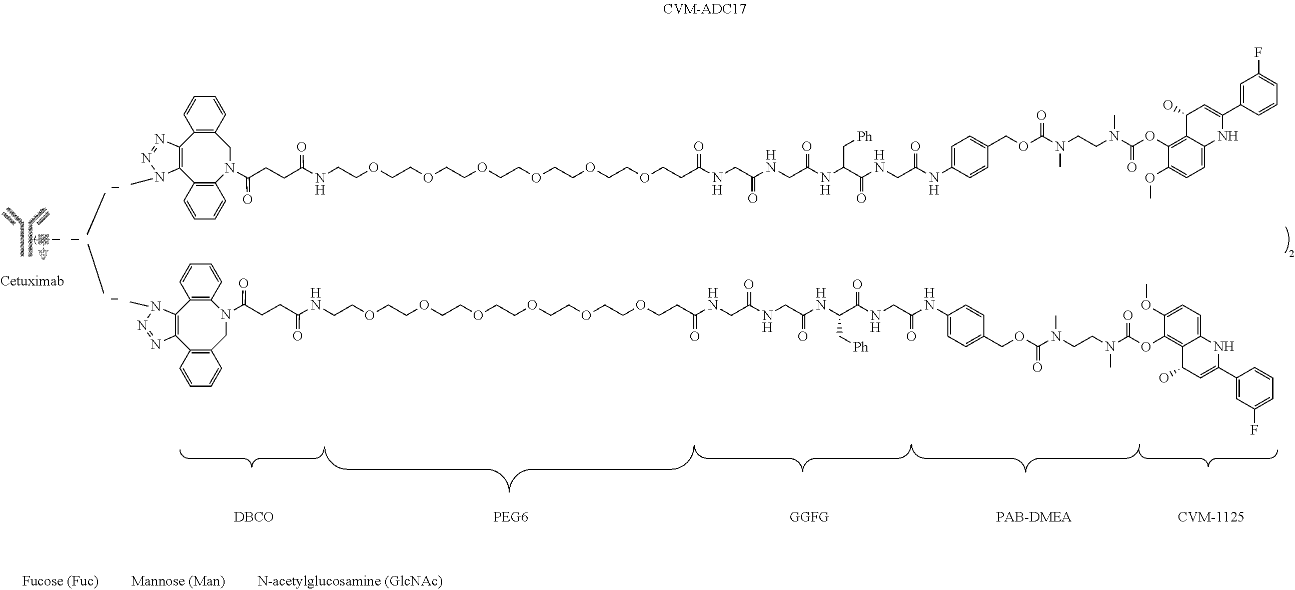

The antibody-drug conjugation of CVM-ADC19 for the present disclosure can be prepared by the following Scheme 14.

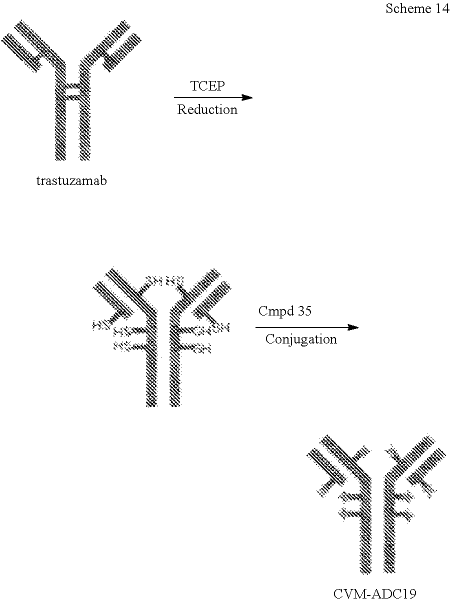

### Preparation of ADC for CVM-ADC19

a) Take 0.181.6 mL of antibody (11.014 mg/mL, 2 mg, in PBS7.2 (1×)/1 mM EDTA) into a new 1 mL vial.
b) Use 0.0184 mL PBS7.2 (1×)/1 mM EDTA to adjust the final antibody reaction concentration to 10 mg/mL.
c) Add 0.006 mL of TECP (10 mM in PBS7.2 (1×)/1 mM EDTA, 4.5 equivalents) to the 1 mL vial.
d) Incubate the solution at 37° C. and shake for 2 hours.
e) Add 0.0289 mL of DMSO to make up 17% of the total volume containing DMSO.
f) Add 0.0133 mL of compound 35 (i.e., MC-vc-PAB-DMEA-CVM-1125)(10 mM in DMSO, 10 equivalents) to the 1 mL vial.
g) Incubate the solution at room temperature and shake for 2 hours.

### Purification of ADC for CVM-ADC19

a) Use a 30 kDa centrifugal filter to replace the solution with 1×PBS7.2 buffer (centrifuge at 4,500 rcf, 4° C. for 20 minutes) and repeat this step 4 times.
b) Filter the solution through a 0.22 μm filter (Yield: >99% and DAR: 7.58).

The structure of CVM-ADC19 is shown below:

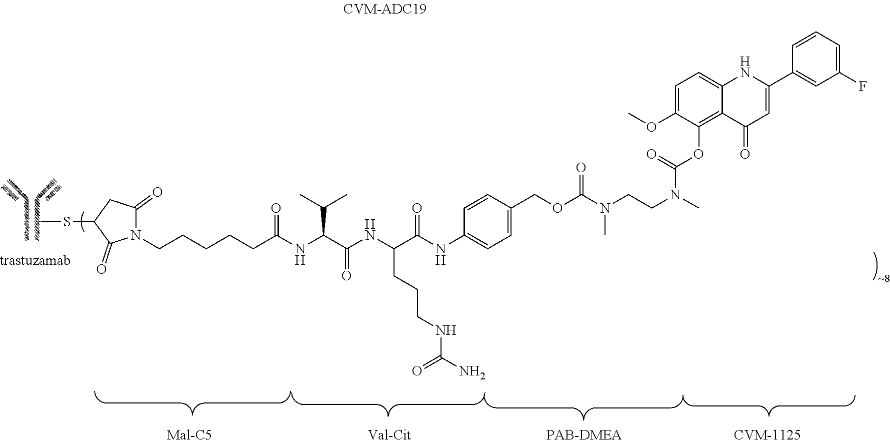 [SMILES: COc1ccc2[nH]c(-c3cccc(F)c3)cc(=O)c2c1OC(=O)N(C)CCN(C)C(=O)OCc1ccc(NC(=O)C(CCCNC(N)=O)NC(=O)[C@@H](NC(=O)CCCCCN2C(=O)CC(SC)C2=O)C(C)C)cc1 ; "Mal-C5" ; "Val-Cit" ; "PAB-DMEA" ; "CVM-1125"]

The antibody-drug conjugation of CVM-ADC20 for the present disclosure can be prepared by the following Scheme 15.

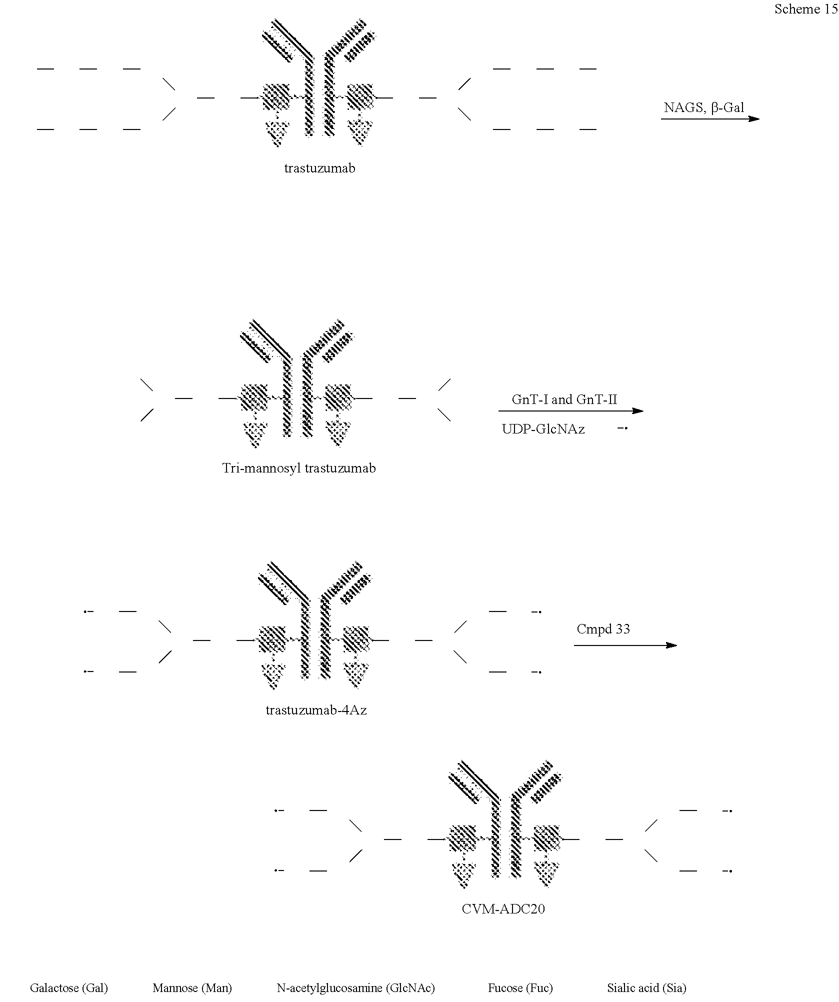

### Preparation of ADC for CVM-ADC20

a) Take 3.874 mL of antibody (6.454 mg/mL, 25 mg) into a new 50 mL tube.
b) Use 1.126 mL 25 mM pH 7.5 MOPS to adjust the final antibody reaction concentration to 5 mg/mL.
c) Add 0.333 mL of DMA to make up 20% of the total volume containing DMA.
d) Add 0.667 mL of compound 33 (i.e., DBCO-PEG6-vc-PAB-DMEA-CVM-1125)(10 mM in DMA, 40 equivalents) to the 50 mL tube.
e) Incubate the solution at 37° C. and shake for 18 hours.

### Purification of ADC for CVM-ADC20

a) Use a 30 kDa centrifugal filter to replace the solution with 25 mM pH 7.5 MOPS buffer (centrifuge at 4500 rcf, 4° C. for 30 minutes) and repeat this step 4 times.
b) Filter the solution through a 0.22 μm filter (Yield: >99% and DAR: 3.92).

The structure of CVM-ADC20 is shown below:

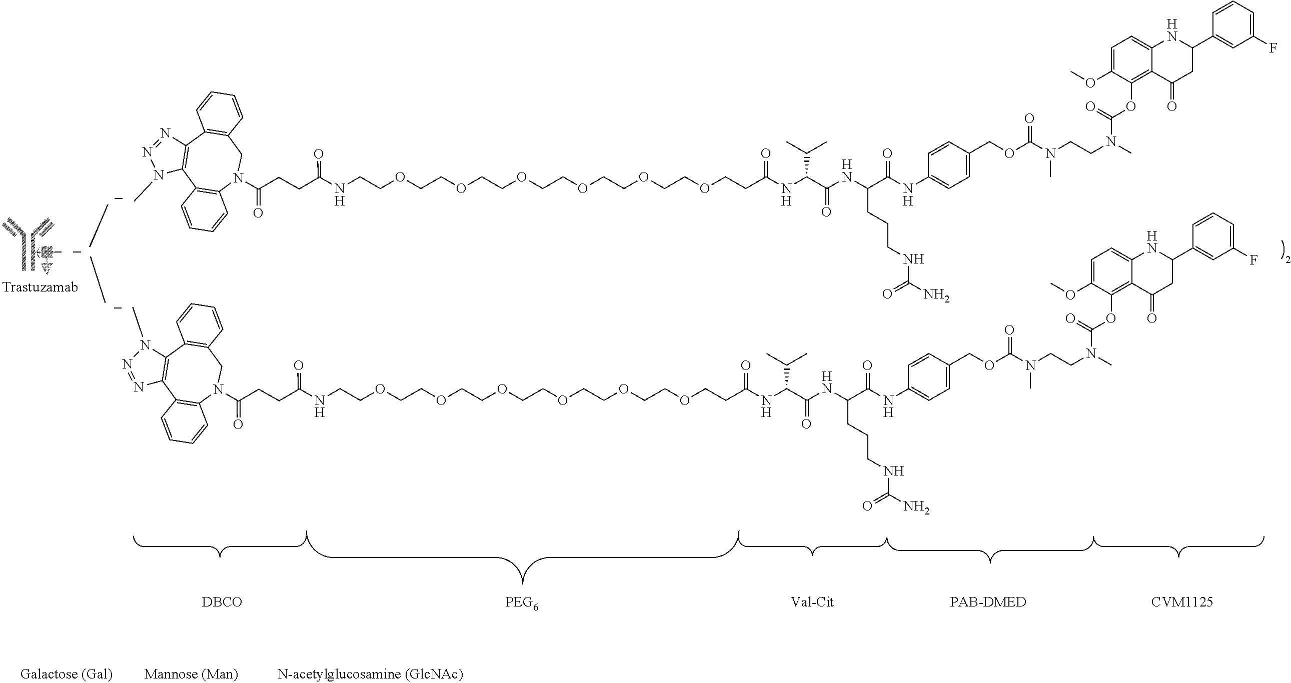

The antibody-drug conjugation of CVM-ADC21 for the present disclosure can be prepared by the following Scheme 16.

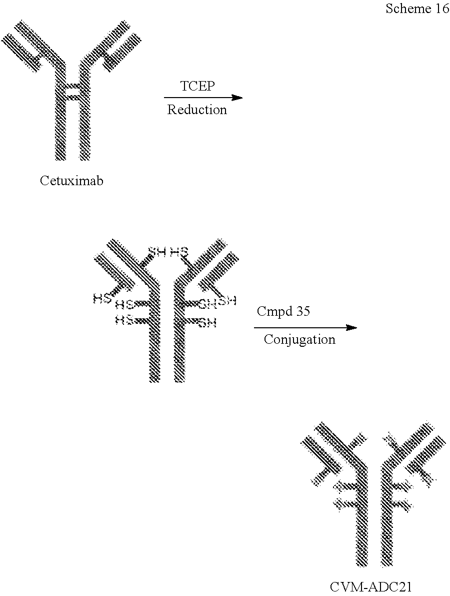

### Preparation of ADC for CVM-ADC21

a) Preparation of antibody solution
   Exchange the buffer of Erbitux antibody to working buffer (1×PBS, 1 mM EDTA pH7.2)
b) Preparation of reducing agent solution
   Add 0.259 mg TCEP into 0.09 mL working buffer and adjust the concentration of TCEP solution to 10 mM.
c) Preparation of linker-payload solution
   Add 2.0 mg compound 35 (i.e., MC-vc-PAB-DMEA-CVM-1125) into 0.200 mL DMSO and adjust the concentration of MC-vc-PAB-DMEA-CVM-1125 solution to 10 mM.
d) Antibody reduction
   Add 0.09 mL reducing agent solution to 3.0 mL antibody solution in a sterile 50 mL sample vail
   Move the antibody solution into an incubator shaker at 37° C. at a rotation speed of 150 rpm and incubate for 2 hours.
   Add 0.433 mL DMSO into reduced antibody solution, to make up 17% of the total volume containing DMA.
   Add 0.200 mL linker-payload solution into reduced antibody solution. Move the antibody solution mixture into an incubator shaker at 25° C. at a rotation speed of 150 rpm and incubate for 2 hours.

### Purification of ADC for CVM-ADC21

a) Use a 30 kDa centrifugal filter to replace the solution with 25 mM MOPS pH 7.5 buffer (centrifuge at 4000×g, 4° C. for 30 minutes) and repeat this step 3 times.
b) Filter the solution through a 0.22 μm filter (Yield: 72% and DAR: 7.37).

The structure of CVM-ADC21 is shown below:

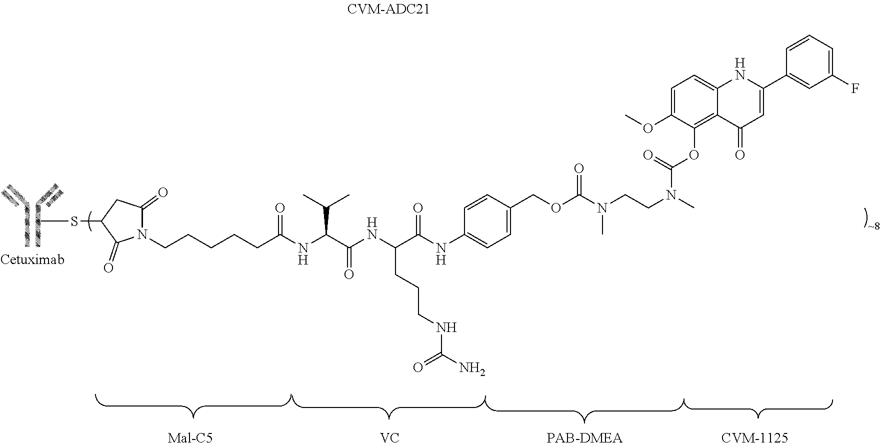

The antibody-drug conjugation of CVM-ADC22 for the present disclosure can be prepared by the following Scheme 17.

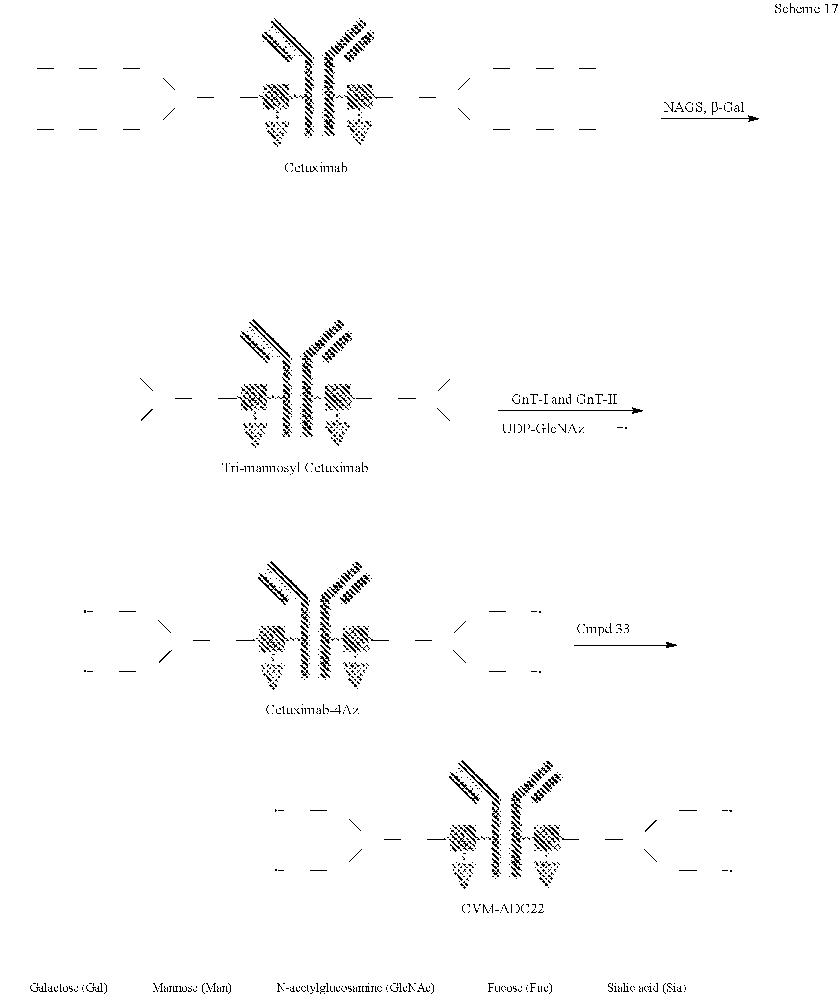

### Preparation of ADC for CVM-ADC22

a) Take 2.502 mL of antibody (7.994 mg/mL, 20 mg) into a new 50 mL tube.
b) Use 1.498 mL 25 mM pH7.5 MOPS to adjust the final antibody reaction concentration to 5 mg/mL.
c) Add 0.267 mL of DMA to make up 20% of the total volume containing DMA.
d) Add 0.533 mL of compound 33 (i.e., DBCO-PEG6-vc-PAB-DMEA-CVM-1125)(10 mM in DMA, 40 equivalents) to the 50 mL tube.
e) Incubate the solution at 37° C. and shake for 18 hours.

### Purification of ADC for CVM-ADC22

a) Use a 30 kDa centrifugal filter to replace the solution with 25 mM pH 7.5 MOPS (centrifuge at 5,000 rpm, 4° C. for 40 minutes) and repeat this step 4 times.
b) Filter the solution through a 0.22 μm filter (Yield: 108% and DAR: 3.98).

The structure of CVM-ADC22 is shown below:

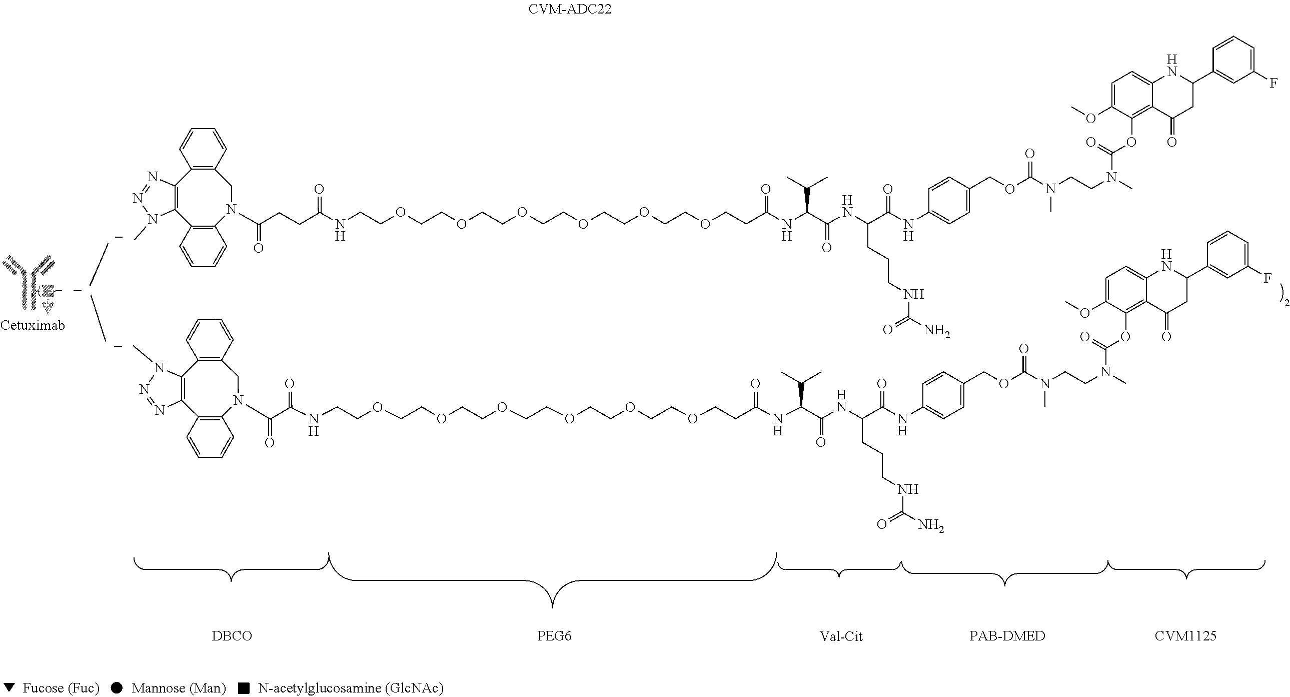

The antibody-drug conjugation of CVM-ADC23 for the present disclosure can be prepared by the following Scheme 18.

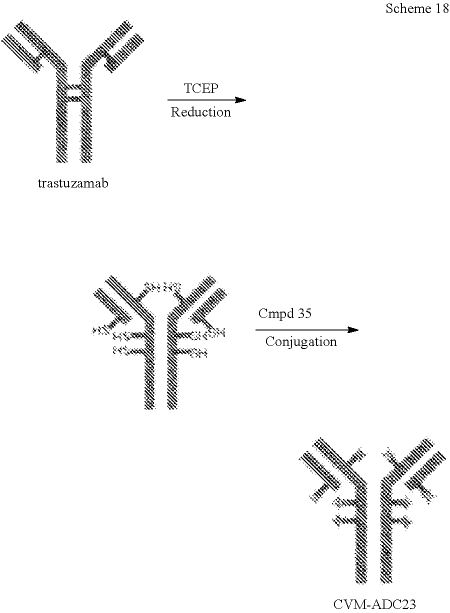

### Preparation of ADC for CVM-ADC23

a) Take 0.1822 mL of antibody (10.978 mg/mL, 2 mg, in 25 mM pH 7.5 MOPS/1 mM EDTA) into a new 1 mL vial.
b) Use 0.0178 mL 25 mM pH 7.5 MOPS/1 mM EDTA to adjust the final antibody reaction concentration to 10 mg/mL.
c) Add 0.00667 mL of TECP (10 mM in 25 mM pH 7.5 MOPS/1 mM EDTA, 2.5 equivalents) to the 1 mL vial.
d) Incubate the solution at 37° C. and shake for 2 hours.
e) Add 0.035 mL of DMSO to make up 17% of the total volume containing DMSO.
f) Add 0.00667 mL of compound 35 (i.e., MC-vc-PAB-DMEA-CVM-1125)(10 mM in DMSO, 5 equivalents) to the 1 mL vial.
g) Incubate the solution at room temperature and shake for 2 hours.

### Purification of ADC for CVM-ADC23

a) Use a 30 kDa centrifugal filter to replace the solution with 25 mM pH 7.5 MOPS buffer (centrifuge at 4500 rcf, 4° C. for 20 minutes) and repeat this step 4 times.
b) Filter the solution through a 0.22 μm filter (Yield: >99% and DAR: 3.99).

The structure of CVM-ADC23 is shown below:

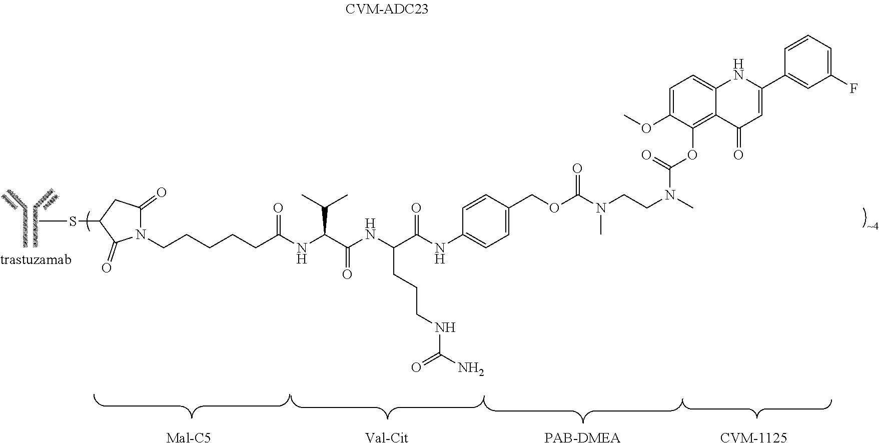

The antibody-drug conjugates of the present disclosure were prepared by connecting the compounds 15, 26, 29, 33, 35 and 38 provided in Example 1 to trastuzumab or cetuximab, respectively. The antibody-drug conjugated products provided in Example 2 are summarized in Table 5.

TABLE 5

| No. | ADC Name | Antibody (Ab) | Linker-Payload | DAR | Purity |
|---|---|---|---|---|---|
| 1 | CVM-ADC01 | Trastuzumab (Her2 Ab) | Compound 15 (-DBCO-PEG6-va-PAB-DMEA-CVM-1125) | 3.6 | 99.02% |
| 2 | CVM-ADC02 | Trastuzumab (Her2 Ab) | Compound 38 (-DBCO-PEG6-branched- | 7.86 | 91.82% |
| | | | (PEG4-va-PAB-DMEA-CVM-1125)₂ | | |
| 3 | CVM-ADC10 | Cetuximab (EGFR Ab) | Compound 15 (-DBCO-PEG6-va-PAB-DMEA-CVM-1125) | 3.41 | 99.29% |
| 4 | CVM-ADC14 | Trastuzumab (Her2 Ab) | Compound 26 (-DBCO-PEG6-GGFG-PAB-DMEA-CVM-1125) | 3.56 | 91.41% |
| 5 | CVM-ADC17 | Cetuximab (EGFR Ab) | Compound 26 (-DBCO-PEG6-GGFG-PAB-DMEA-CVM-1125) | 3.8 | 92.00% |
| 6 | CVM-ADC19 | Trastuzumab (Her2 Ab) | Compound 35 (-MC-vc-PAB-DMEA-CVM-1125) | 7.58 | 61.24% |
| 7 | CVM-ADC20 | Trastuzumab (Her2 Ab) | Compound 33 (-DBCO-PEG6-vc-PAB-DMEA-CVM-1125) | 3.92 | 97.59% |
| 8 | CVM-ADC21 | Cetuximab (EGFR Ab) | Compound 35 (-MC-vc-PAB-DMEA-CVM-1125) | 7.37 | 94.70% |
| 9 | CVM-ADC22 | Cetuximab (EGFR Ab) | Compound 33 (DBCO-PEG6-vc-PAB-DMEA-CVM-1125) | 3.98 | 89.60% |
| 10 | CVM-ADC23 | Trastuzumab (Her2 Ab) | Compound 35 (-MC-vc-PAB-DMEA-CVM-1125) | 3.99 | 97.68% |

The antibody-drug conjugates thus provided are summarized in Table 6.

TABLE 6

| | | | ADC Product | | |
|---|---|---|---|---|---|
| Sample | | | (Ab—[L—(D)ₚ]_{q}; p = 1-3; | | Linker-Payload |
| No. | ADC Product Name | DAR | q = 1-8) | Antibody(Ab) | (L—(D)ₚ; p = 1-3) |
| 1 | CVM-ADC01 | 3.6 | p = 1, q = 4 | Trastuzumab | Compound 15 |
| 2 | CVM-ADC02 | 7.86 | P = 2, q = 4 | Trastuzumab | Compound 38 |
| 3 | CVM-ADC10 | 3.41 | p = l, q = 4 | Cetuximab | Compound 15 |
| 4 | CVM-ADC14 | 3.56 | p = 1, q = 4 | Trastuzumab | Compound 26 |
| 5 | CVM-ADC17 | 3.8 | p = 1, q = 4 | Cetuximab | Compound 26 |
| 6 | CVM-ADC19 | 7.58 | p = 1, q = 8 | Trastuzumab | Compound 35 |
| 7 | CVM-ADC20 | 3.92 | p = 1, q = 4 | Trastuzumab | Compound 33 |
| 8 | CVM-ADC21 | 7.37 | p = 1, q = 8 | Cetuximab | Compound 35 |
| 9 | CVM-ADC22 | 3.98 | p = 1, q = 4 | Cetuximab | Compound 33 |
| 10 | CVM-ADC23 | 3.99 | p = 1, q = 4 | Trastuzumab | Compound 35 |

### Example 3: Tumor Growth Inhibition Effect of CVM-1125 ADC in the In Vivo Xenograft Models

### Experimental Methods and Procedures

The BT-474 and MDA-MB-468 human breast cancer cell lines were cultured in RPMI-1640 and L-15 medium supplementation with 10% heat inactivated fetal bovine serum at 37° C. in an atmosphere of 5% CO₂ in air. 6-8 weeks old BALB/c nude and 7-8 weeks old NOD-SCID mice were inoculated subcutaneously to the right front flank with 1.0×10⁷ and 1.5×10⁷ BT-474 cells, respectively. Seven days prior to BT-474 cells inoculation, each mouse was subcutaneously implanted on the left front flank with an 17β-Estradiol (E2) pellet using a trocar needle. 6-8 weeks old BALB/c nude mice were inoculated subcutaneously to the right flank with 10×10⁶ MDA-MB-468 cells. Tumors were monitored with digimatic calipers, and the tumor volume was expressed using the formula: TV=(w²×1)/2; where w=width and l=length in diameter (mm) of the tumor. When the mean tumor volume (MTV) reached approximately 100˜150 mm³, tumor-bearing mice were randomly grouped and administrated with ADC or vehicle solution on the same study day, which was denoted as Day 0. Tables 7-10 present the summary of experimental design. All test articles and vehicle were Intravenously (IV) administered once per week (QW) to tumor-bearing mice for 2 or 3 weeks in a volume of 10 mL/kg body weight (BW). Group 1 animals in both experiments received the same volume of 25 mM Na-Citrate pH 6.5 buffer, served as vehicle control group for calculation of tumor growth inhibition (TGI) rate.

TABLE 7
Experimental design in BT-474 human breast cancer xenograft
model in BALB/c nude mice

| | Animal | | Dose | Dosing route and |
|---|---|---|---|---|
| Group | number | Treatment | (mg/kg) | frequency |
| 1 | 8 | Vehicle | — | IV, QW for 3 weeks |
| 2 | 8 | CVM-ADC01 | 5 | IV, QW for 3 weeks |
| 3 | 8 | CVM-ADC20 | 5 | IV, QW for 3 weeks |
| 4 | 8 | CVM-ADC23 | 5 | IV, QW for 3 weeks |

TABLE 8
Experimental design in BT-474 human breast cancer xenograft
model in NOD-SCID mice

| | Animal | | Dose | Dosing route |
|---|---|---|---|---|
| Group | number | Treatment | (mg/kg) | and frequency |
| 1 | 4 | Vehicle | — | IV, QW for 3 weeks |
| 2 | 4 | Herceptin | 5 | IV, QW for 3 weeks |
| 3 | 4 | CVM-ADC01 | 5 | IV, QW for 3 weeks |
| 4 | 4 | CVM-ADC01 | 10 | IV, QW for 3 weeks |

TABLE 9
Experimental design in BT-474 human breast cancer model
xenograft model in NOD-SCID mice

| | Animal | | Dose | Dosing route |
|---|---|---|---|---|
| Group | number | Treatment | (mg/kg) | and frequency |
| 1 | 8 | Vehicle | — | IV, QW for 3 weeks |
| 2 | 8 | CVM-ADC01 | 5 | IV, QW for 3 weeks |
| 3 | 8 | CVM-ADC02 | 5 | IV, QW for 3 weeks |

TABLE 10
Experimental design in MDA-MB-468 human breast cancer
xenograft model in BALB/c nude mice

| | Animal | | Dose | Dosing route |
|---|---|---|---|---|
| Group | number | Treatment | (mg/kg) | and frequency |
| 1 | 8 | Vehicle | — | IV, QW for 2 weeks |
| 2 | 8 | Cetuximab | 5 | IV, QW for 2 weeks |
| 3 | 8 | CVM-ADC10 | 5 | IV, QW for 2 weeks |

Body weight change was calculated as the percentage increase in body weight compared to the initial body weight on Day 0. TGI rate was calculated using the following formula: % TGI=[1−(T/C)]×100%; where T and C represent the mean TV % of the treatment group and the vehicle control group, respectively. Data was expressed as Mean±SEM. Comparisons between two groups were performed using Student's t test. The study was terminated on Day 28 post-treatment and all animals were euthanized with CO₂. The tumors of individual mice were collected and weighed. The animals were euthanized when the group mean tumor volume over 2,000 mm³ or the body weight loss exceeds 20% from the initial treatment. All animal procedures were performed under a protocol approved by the Institutional Animal Care and Use Committee (IACUC)

### In Vivo Efficacy of ADCs Using CVM-1125 as Payload on BT-474 Human Breast Cancer Model

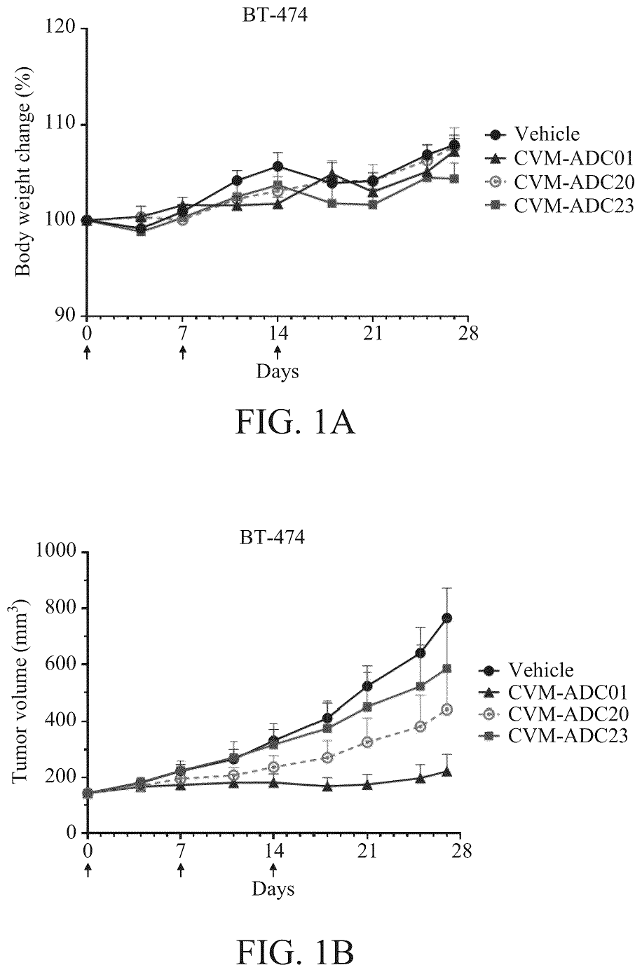

CVM-1125 targets TRAP1 and suppresses tumor cell growth via inhibiting vasculogenic mimicry and promoting apoptosis. To assess the potential of CVM-1125 as a cytotoxic payload of antibody-drug conjugates, a series of CVM-1125-based ADCs (CVM-ADC01, CVM-ADC20, CVM-ADC23) composed of a HER2-specific humanized antibody (Herceptin) with conjugated CVM-1125 via various cleavable linkers at Drug-to-Antibody Ratio (DAR) of 4 were evaluated in HER2-positive BT-474 xenograft model (Table 5 and 7). No unscheduled animal death or clinical signs were observed during the study. The mean percentage BW changes over time are shown in FIG. 1A. The arrows refer to the dosing days mice received vehicle or ADCs. As shown in FIG. 1A, no significant BW loss was observed in all mice administration of vehicle or 5 mg/kg of test articles in this study, and there was no significant difference in weight changes between all treatment groups. These results indicate that animals are well tolerated with all CVM-1125-based ADCs.

The TGI rates of BT-474 human breast cancer in individual treatment group are summarized in Table 11, the average tumor growth curves are shown in FIG. 1B.

TABLE 11

| Treatment | | D4 | D7 | D11 | D14 |
|---|---|---|---|---|---|
| CVM-ADC01 | % TGI | 7.7 | 22.1 | 31.8 | 45.2* |
| 5 mg/kg | P value | 0.5532 | 0.1639 | 0.0845 | 0.0110 |
| CVM-ADC20 | % TGI | 5.7 | 12.2 | 21.9 | 28.6 |
| 5 mg/kg | P value | 0.6111 | 0.3879 | 0.2081 | 0.1276 |
| CVM-ADC23 | % TGI | −1.5 | −0.9 | −2.1 | 4.3 |
| 5 mg/kg | P value | 0.9113 | 0.9652 | 0.9341 | 0.8713 |
| Treatment | | D18 | D21 | D25 | D27 |
| CVM-ADC01 | % TGI | 59.2** | 66.7** | 69.2** | 71.1** |
| 5 mg/kg | P value | 0.0011 | 0.0007 | 0.0008 | 0.0006 |
| CVM-ADC20 | % TGI | 34.4 | 37.7 | 40.5 | 42.4 |
| 5 mg/kg | P value | 0.0969 | 0.1012 | 0.0897 | 0.0792 |
| CVM-ADC23 | % TGI | 9.0 | 14.0 | 18.5 | 23.5 |
| 5 mg/kg | P value | 0.7396 | 0.6161 | 0.5084 | 0.3959 |

P value compared to Vehicle control group. *P < 0.05 and **P < 0.01.

As shown in Table 11 and FIG. 1B, all of the CVM-1125-based ADCs (CVM-ADC01, CVM-ADC20, CVM-ADC23) can provide an anti-tumor efficacy. CVM-ADC01, which consisted of VA linker, even exhibited a significant anti-tumor efficacy on Day 14 (P=0.0011) to Day 27 (P=0.0006), compared to vehicle group. These results indicate that the antibody-drug conjugate of the present disclosure can effectively inhibit tumor growth. These results indicate CVM-1125 is a potential toxic payload of ADC in tumor inhibition.

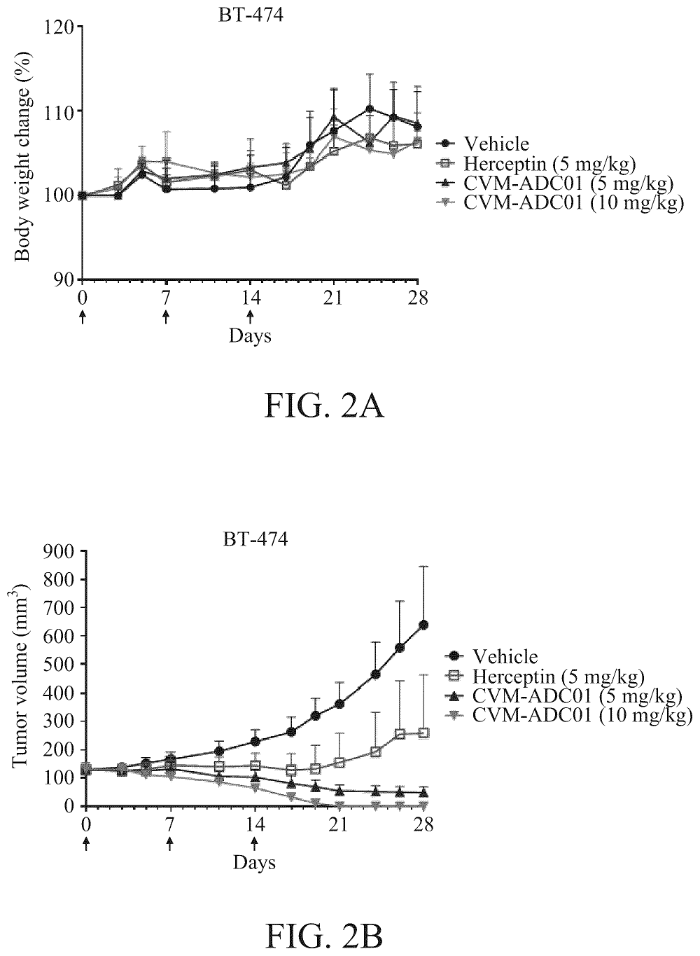

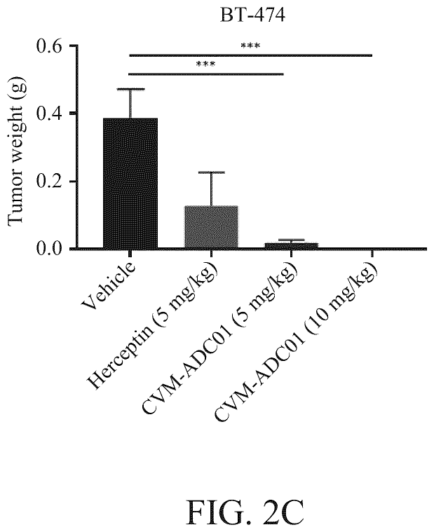

The dose range efficacy study of CVM-ADC01 was performed in BT-474 model. As shown in FIG. 2A, no significant BW loss was observed in all mice administration of vehicle or CVM-ADC01 at 5 and 10 mg/kg. Also, no abnormal clinical signs were observed during the study.

The TGI rates of BT-474 human breast cancer in individual treatment group are summarized in Table 12, the average tumor growth curves and tumor weight are shown in FIG. 2B, and the average tumor weights on Day 28 are shown in FIG. 2C.

TABLE 12

| Treatment | | D 3 | D 5 | D 7 | D 11 | D 14 |
|---|---|---|---|---|---|---|
| Herceptin | % TGI | 8.7 | 13.6 | 13.8 | 28.5 | 39.6 |
| 5 mg/kg | P value | 0.6631 | 0.5216 | 0.6074 | 0.3198 | 0.2141 |
| CVM-ADC01 | % TGI | 10.2 | 17.5 | 20.6 | 44.4 | 55.8* |
| 5 mg/kg | P value | 0.5474 | 0.3089 | 0.3261 | 0.0676 | 0.0486 |
| CVM-ADC01 | % TGI | 6.3 | 26.2 | 35.7 | 54.1* | 70.0** |
| 10 mg/kg | P value | 0.7384 | 0.1785 | 0.0692 | 0.0246 | 0.0085 |

| Treatment | | D 17 | D 19 | D 21 | D 24 | D 26 | D 28 |
|---|---|---|---|---|---|---|---|
| Herceptin | % TGI | 56.2 | 65.1 | 63.4 | 63.4 | 59.1 | 63.1 |
| 5 mg/kg | P value | 0.1383 | 0.1203 | 0.1541 | 0.1854 | 0.2701 | 0.2382 |
| CVM-ADC01 | % TGI | 69.6* | 78.6** | 85.5** | 88.9* | 91.0* | 92.4* |
| 5 mg/kg | P value | 0.0225 | 0.0091 | 0.0067 | 0.0125 | 0.0225 | 0.0298 |
| CVM-ADC01 | % TGI | 87.2** | 97.2** | 100.0** | 100.0** | 100.0* | 100.0* |
| 10 mg/kg | P value | 0.0049 | 0.0025 | 0.0025 | 0.0068 | 0.0149 | 0.0215 |

P value compared to Vehicle control group.
*P < 0.05 and
**P < 0.01.

As shown in Table 12 and FIGS. 2B and 2C, significant anti-tumor efficacy was observed in 5 mg/kg CVM-ADC01 group on Day 14 (P=0.0486) to Day 28 (P=0.0298), and in 10 mg/kg CVM-ADC01 group on Day 11 (P=0.0246) to Day 28 (P=0.0215), compared to vehicle group, respectively. Moreover, 10 mg/kg of CVM-ADC01 completely suppressed BT-474 tumor growth from Day 21 until Day 28. The TGI rate on Day 28 was 92.4% in 5 mg/kg CVM-ADC01 group, and 100% for 10 mg/kg CVM-ADC01, respectively. Moreover, on Day 28, both 5 mg/kg and 10 mg/kg CVM-ADC01 groups, but not 5 mg/kg Herceptin group with 63.1% TGI (P=0.2382), had significantly lower tumor weight than the vehicle control group (P<0.005). In contrast, though 5 mg/kg Herceptin showed 63.1% TGI on Day 28, the antitumor efficacy thereof is not statistically significant as compared to Vehicle control. The average tumor weights of both 5 mg/kg and 10 mg/kg CVM-ADC01 groups have significant lower tumor weight than Vehicle control (P<0.005), but not Herceptin groups. These results indicate that the antibody-drug conjugate of the present disclosure can effectively inhibit tumor growth, and the inhibiting effect of CVM-ADC01 is better than the reference drug Herceptin, showing CVM-1125 is potential toxic payload of ADC in tumor inhibition.

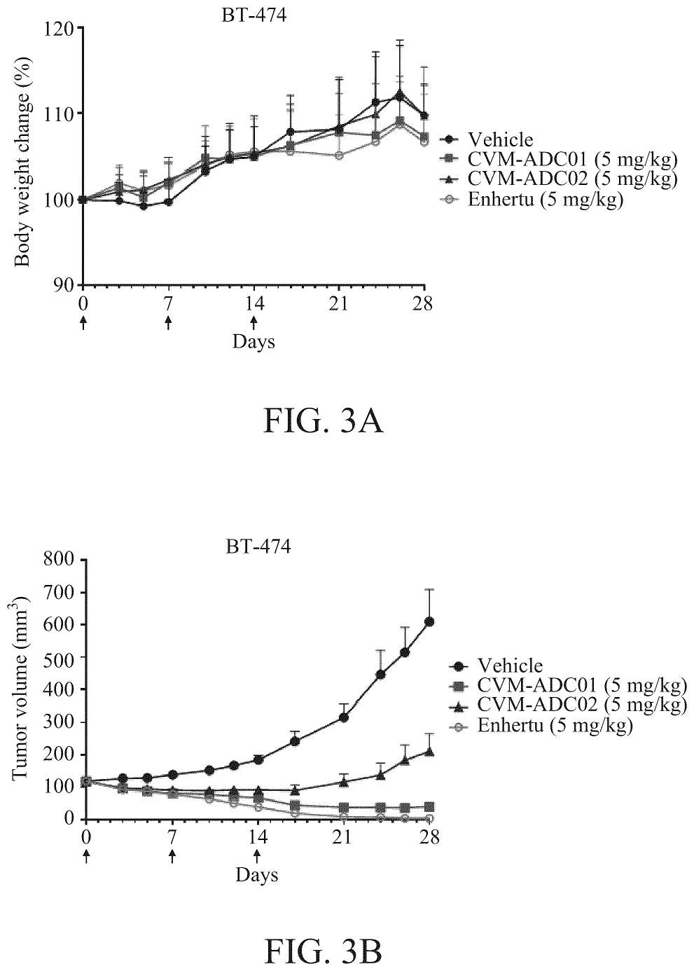

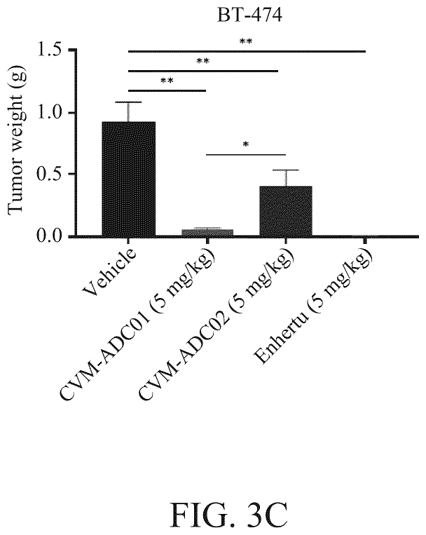

To optimize the efficacy of ADC with different DAR, the tumor inhibitory effects of CVM-ADC02 at DAR8 and CVM-ADC01 at DAR4 were evaluated in BT-474 xenograft model. As shown in Table 12 and FIGS. 3A-3C, all ADCs had no impact on the body weight (FIG. 3A). 5 mg/kg CVM-ADC01 and HER2-targeted reference drug (Enhertu) exhibited anti-tumor efficacy with TGI % at 94.5% (P<0.01) and 99.7% (P<0.01), compared to vehicle group, respectively. However, 5 mg/kg CVM-ADC02 exhibited TGI at 66.9% (Table 13 and FIG. 3B). 5 mg/kg CVM-ADC01 also showed a promising efficacy in lowering the tumor weight, compared to 5 mg/kg CVM-ADC02 (FIG. 3C). These results indicated CVM-ADC01 with optimized CVM-1125 payload number at DAR4, rather than CVM-ADC02 at DAR8, showed comparable efficacy to Enhertu.

TABLE 13

| Treatment | | D 3 | D 5 | D 7 | D 10 | D 12 | D 14 |
|---|---|---|---|---|---|---|---|
| CVM-ADC01 | % TGI | 25.3 | 31.9** | 41.4** | 48.8** | 56.8** | 63.0** |
| 5 mg/kg | P value | 0.0675 | 0.0034 | 0.0002 | 3.84E−05 | 1.07E−06 | 2.36E−06 |
| CVM-ADC02 | % TGI | 24.2 | 26.9* | 35.2** | 42.1** | 46.2** | 51.0** |
| 5 mg/kg | P value | 0.1168 | 0.0247 | 0.0045 | 0.0031 | 0.0011 | 0.0005 |
| Enhertu | % TGI | 27.7* | 32.6** | 44.3** | 59.5** | 71.1** | 79.5** |
| 5 mg/kg | P value | 0.0182 | 0.0044 | 0.0004 | 1.54E−05 | 3.81E−07 | 3.08E−07 |

| Treatment | | D 17 | D 21 | D 24 | D 26 | D 28 |
|---|---|---|---|---|---|---|
| CVM-ADC01 | % TGI | 81.5** | 88.1** | 91.6** | 92.8** | 93.2** |
| 5 mg/kg | P value | 1.98E−05 | 1.39E−05 | 9.5E−05 | 2.37E−05 | 5.81E−05 |
| CVM-ADC02 | % TGI | 62.9** | 63.2** | 70.0** | 65.4** | 66.2** |
| 5 mg/kg | P value | 0.0010 | 0.0067 | 0.0068 | 0.0108 | 0.0062 |
| Enhertu | % TGI | 92.3** | 97.2** | 98.4** | 99.2** | 99.4** |
| 5 mg/kg | P value | 5.04E−06 | 4.49E−06 | 4.55E−05 | 1.12E−05 | 2.89E−05 |

P value compared to Vehicle control group.
*P < 0.05 and
**P < 0.01.

### In Vivo Efficacy of CVM-ADC10 on MDA-MB-468 Human Breast Cancer Model

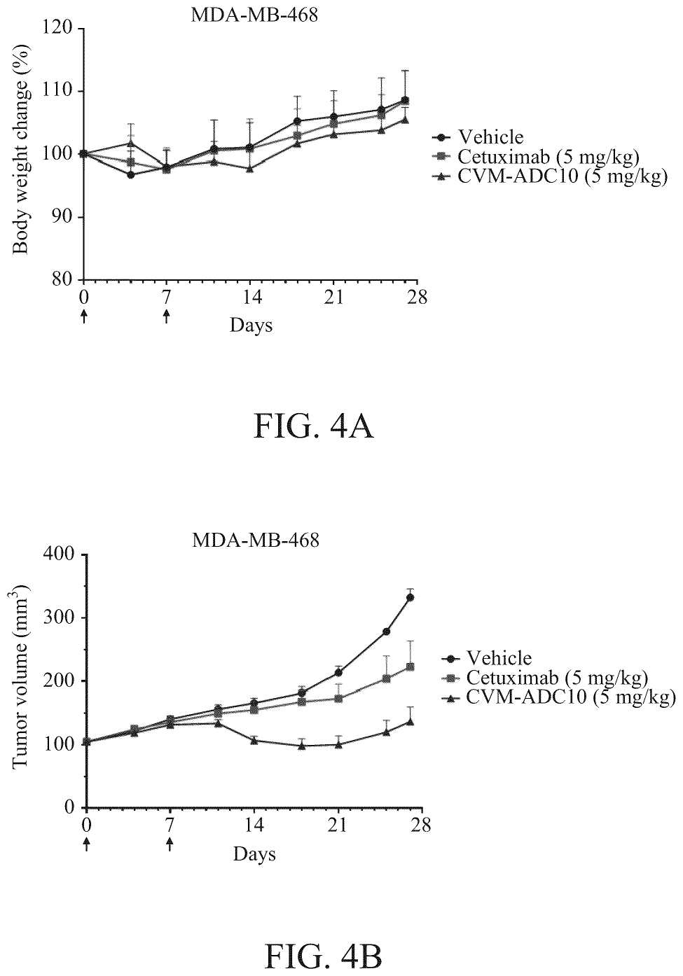

The EGFR-targeted CVM-ADC10 ADC covalently attached with CVM-1125 conjugated with an anti-EGFR antibody, Cetuximab, via VA linker was further evaluated in in vivo efficacy study. 5 mg/kg CVM-ADC10 and Cetuximab were administrated in EGFR-positive MDA-MB-468 xenograft model. The mean percentage of BW changes over time are shown in FIG. 4A. Also, no death or clinical signs were observed in the treated animals during the study. The arrows in FIG. 4B refer to the dosing days mice received vehicle, test article or reference drug (Cetuximab).

As shown in Table 14 and FIG. 4B, a significant anti-tumor efficacy in MDA-MB-468 xenograft model was observed in CVM-ADC10 group on Day 14 (P<0.01) to Day 21 (P=0.014) and positive trend on Day 25 (P=0.0616), compared to Cetuximab group. The TGI rate on Day 27 was 58.75% in CVM-ADC10 group, compared to 33.01% in Cetuximab group (P=0.0915). These results indicate that ADC using CVM-1125 payload conjugating other targeting antibody, like anti-EGFR antibody, is efficacious to achieve enhanced anti-tumor effects without an increase of body weight or toxicity (FIG. 4A).

TABLE 14

| Treatment | | D4 | D7 | D11 | D14 |
|---|---|---|---|---|---|
| Cetuximab | % TGI | −2.15 | 3.32 | 4.34 | 6.64 |
| 5 mg/kg | P value | 0.7788 | 0.6563 | 0.5945 | 0.4951 |
| CVM-ADC10 | % TGI | 2.64 | 6.28 | 14.23* | 35.61** |
| 5 mg/kg | P value | 0.7137 | 0.3287 | 0.0309 | 6.31E−05 |
| Cetuximab vs CVM-ADC10 | P value | 0.4873 | 0.7072 | 0.2033 | 0.0074 |
| Treatment | | D18 | D21 | D25 | D27 |
| Cetuximab | % TGI | 7.50 | 18.77 | 26.87 | 33.01* |
| 5 mg/kg | P value | 0.5150 | 0.1243 | 0.0613 | 0.0241 |
| CVM-ADC10 | % TGI | 45.96** | 52.92** | 56.68** | 58.75** |
| 5 mg/kg | P value | 7.41E−05 | 1.56E−05 | 1.05E−06 | 4.45E−06 |
| Cetuximab vs CVM-ADC10 | P value | 0.0054 | 0.0142 | 0.0616 | 0.0915 |

P value compared to Vehicle control group or Cetuximab vs CVM-ADC10, *P < 0.05 and ** P < 0.01.

It can be seen from the examples above, the linker-payload of the present disclosure can be synthesized through site-specific reactions. The synthesis of the linking group (L) to the drug (D) at R₅ site (OH group) proved challenging due to the instability of the phenyl carbonate activation intermediate. This intermediate exhibited high susceptibility to decomposition or reconversion back into original drug under various reaction conditions. To address this issue, a stable [1,3]dioxin-2-one structure was synthesized using 4-nitrobenzyl chloroformate as the activation reagent. The linker-payload thus obtained can be further conjugated with an antibody so as to provide an antibody-drug conjugate. No observable aggregates were detected in the antibody-drug conjugate, and the antibody-drug conjugate is well tolerated. In vivo pharmacology study results indicated that the antibody-drug conjugate showed significant antitumor efficacy, and even exhibited profound antitumor efficacy in the BT-474 human breast cancer model with TGI 92.4%, compared to Herceptin antibody group with TGI 63.1%, as well as in the MDA-MB-468 human breast cancer model with 58.75% TGI rates, compared to Cetuximab antibody with TGI 33.01%. These studies demonstrate the linker-payload and antibody-drug conjugate of the present disclosure are useful for treating cancer.

While some of the embodiments of the present disclosure have been described in detail above, it is, however, possible for those of ordinary skill in the art to make various modifications and changes to the particular embodiments shown without substantially departing from the teaching and advantages of the present disclosure. Such modifications and changes are encompassed in the scope of the present disclosure as set forth in the appended claims.

What is claimed is:

1. A linker-payload having a structure of L-(D)ₚ,
wherein L is a linking group;
p is an integer selected from 1 to 3;
D is a drug having a structure of the following Formula (I): 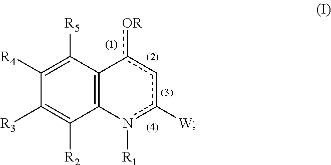
wherein the linking group is connected to the drug at R₅;
R is absent or present, and wherein R, if present, is hydrogen, P(═O)(OH)₂, P(═O)(O(C₁-C₁₈)alkylene(C₆-C₂₀)aryl)₂, P(═O)(OH)(OM), P(═O)(OM)₂, P═O(O₂M), S(═O)(OH)₂, S(═O)(O(C₁-C₁₈)alkylene(C₆-C₂₀)aryl)₂, S(═O)(OH)(OM), S(═O)(OM)₂, wherein M is a monovalent or divalent metal ion, or alkylammonium ion;
R₁ is absent or present, and wherein R₁, if present, is hydrogen;
R₂, R₃ and R₄ independently are H, F, Cl, Br, (CH₂)ₙCH₃, (CH₂)ₙOH, O(CH₂)ₙCH₃, O(CH₂)ₙOH, O(CH₂)ₙNR₈R₉, (CH₂) n SH, S(CH₂)ₙCH₃, S(CH₂)ₙSH, S(CH₂)ₙNR₈R₉, (CH₂)ₙNR₈R₉, (CH₂)ₙN, or R₃ and R₄ together is —O(CH₂)ₙO— or —S(CH₂)ₙS—;
R₅ is O;
each of bonds (1) to (4) independently represents a single bond or a double bond, provided that when bond (1) is a single bond, R is present, R₁ is absent, each of bonds (2) and (4) is a double bond, and bond (3) is a single bond; and when bond (1) is a double bond, R is absent, R₁ is present, each of bonds (2) and (4) is a single bond, and bond (3) is a double bond;
W is an aromatic group selected from the group consisting of naphthyl, quinolinyl, benzofuranyl, benzothiophenyl, anthracenyl, and substituted phenyl of formula (Y): 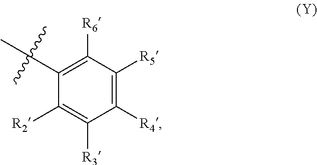
and wherein:
R₂′, R₃′, R₄′, R₅′, and R₆′ are independently H, F, Cl, Br, (CH₂)ₙCH₃, (CH₂ₙ)OH, O—(CH₂)ₙCH₃, O(CH₂)OH, (CH₂ₙ)SH, S—(CH₂)ₙCH₃, S(CH₂)ₙSH, O(CH₂)ₙSH, S(CH₂)ₙOH, (CH₂)ₙNR₈R₉, O(CH₂)ₙNR₈R₉, or S(CH₂)ₙNR₈R₉ or R₃′═OP(═O)(O-benzyl)₂;
wherein R₈ and R₉ are independently H, (CH₂)ₙCH₃, (CH₂ₙ)OH, (CH₂ₙ)SH, (CH₂)ₙN(CₙH₂ₙ₊₁)(CₘH₂ₘ₊₁); and
wherein n and m are each an integer selected from 0 to 4.

2. The linker-payload of claim 1, wherein the linking group has a structure selected from the group consisting of: 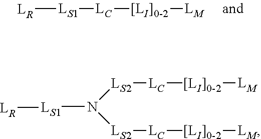
wherein, L_{R} is a reactive group that is capable of reacting with an antibody;
each of L_{S1}, L_{S2}, and L_{S3} is a spacer;
Nis nitrogen;
L_{C} is a cleavable linker or a non-cleavable linker;
L is a self-immolative linker;
L_{M} is a moiety connecting to the R₅ of the drug.

3. The linker-payload of claim 2, wherein L_{R} comprises a thiol, maleimide, haloacetamide, vinyl sulfone, aziridine, azido, alkyne, cyclononyne, cyclooctyne, cyclooctene, triarylphosphine, oxanorbornadiene, diaryltetrazine, aryltetrazine, norbornene, aldehydes, hydroxylamine, hydrazine, NH₂—NH—C(═O)—, ketone, CoA or serine residue.

4. The linker-payload of claim 2, wherein L_{I} is selected from the group consisting of p-aminobenzylcarbamoyl and dimethylethylenediamine.

5. The linker-payload of claim 2, wherein each of the spacer is a PEG chain having 1 to 10 PEG units.

6. The linker-payload of claim 2, wherein L_{C} is a linker comprising a peptide bond, a phosphate bond, a nucleic acid bond, a sugar bond, a disulfide bond, an amide bond, a substituted amide bond in the form of a peptide bond, a thioamide bond, an ester bond, a thioester bond, a vicinal diol bond, or a hemiacetal.

7. The linker-payload of claim 2, wherein L_{M} is selected from the group consisting of —C(═O)—, C₁-C₁₈ alkylene group, —CH₂—CH═CH—, and phenylene group.

8. An antibody-drug conjugate having a structure of Ab-[L-(D)ₚ]_{q},
wherein Ab is an antibody;
L is a linking group;
p is an integer selected from 1 to 3;
q is an integer selected from 1 to 8;
D is a drug having a structure of following Formula (I): 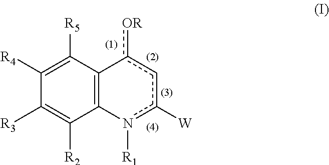
wherein the linking group is connected to the drug at R₅; the linking group is connected between antibody and drug;
R is absent or present, and wherein R, if present, is hydrogen, P(═O)(OH)₂, P(═O)(O(C₁-C₁₈)alkylene(C₆-C₂₀)aryl)₂, P(═O)(OH)(OM), P(═O)(OM)₂, P═O(O₂M), S(═O)(OH)₂, S(═O)(O(C₁-C₁₈)alkylene(C₆-C₂₀)aryl)₂, S(═O)(OH)(OM), S(═O)(OM)₂, wherein M is a monovalent or divalent metal ion, or alkylammonium ion;
R₁ is absent or present, and wherein R₁, if present, is hydrogen;
R₂, R₃ and R₄ independently are H, F, Cl, Br, (CH₂)ₙCH₃, (CH₂)ₙOH, O(CH₂)ₙCH₃, O(CH₂)ₙOH, O(CH₂)ₙNR₈R₉, (CH₂)ₙSH, S(CH₂)ₙCH₃, S(CH₂)ₙSH, S(CH₂)ₙNR₈R₉, (CH₂)ₙNR₈R₉, (CH₂)ₙN, or R₃ and R₄ together is —O(CH₂)ₙO— or —S(CH₂)ₙS—;
R₅ is O;
each of bonds (1) to (4) are a single bond or a double bond, provided that when bond (1) is a single bond, R is present, R₁ is absent, each of bonds (2) and (4) is a double bond, and bond (3) is a single bond; and when bond (1) is a double bond, R is absent, R₁ is present, each of bonds (2) and (4) is a single bond, and bond (3) is a single bond;
W is an aromatic group selected from the group consisting of naphthyl, quinolinyl, benzofuranyl, benzothiophenyl, anthracenyl, and substituted phenyl of formula (Y): 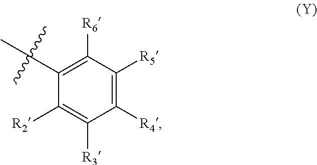
and wherein:
R₂′, R₃′, R₄′, R₅′, and R₆′ are independently H, F, Cl, Br, (CH₂)ₙCH₃, (CH₂ₙ)OH, O—(CH₂)ₙCH₃, O(CH₂)OH, (CH₂ₙ)SH, S—(CH₂)ₙCH₃, S(CH₂)ₙSH, O(CH₂)ₙSH, S(CH₂)ₙOH, (CH₂)ₙNR₈R₉, O(CH₂)ₙNR₈R₉, or S(CH₂)ₙNR₈R₉ or R₃′═OP(═O)(O-benzyl)₂;
wherein R₈ and R₉ are independently H, (CH₂)ₙCH₃, (CH₂ₙ)OH, (CH₂ₙ)SH, (CH₂)ₙN(CₙH₂ₙ₊₁)(CₘH₂ₘ₊₁); and
wherein n and m are each an integer selected from 0 to 4.

9. The antibody-drug conjugate of claim 8, wherein the Drug-to-Antibody Ratio (DAR) of the antibody-drug conjugate is between 1 to 24.

10. The antibody-drug conjugate of claim 8, wherein the linking group has a structure selected from the group consisting of: 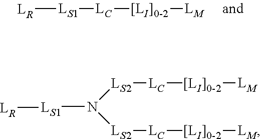
wherein, L_{R} is a reactive group that is capable of reacting with the antibody;
each of L_{S1}, L_{S2}, and L_{S3} is a spacer;
Nis nitrogen;
L_{C} is a cleavable linker or a non-cleavable linker;
L_{I} is a self-immolative linker;
L_{M} is a moiety connecting to the R₅ of the drug.

11. The antibody-drug conjugate of claim 10, wherein L_{R} comprises a thiol, maleimide, haloacetamide, vinyl sulfone, aziridine, azido, alkyne, cyclononyne, cyclooctyne, cyclooctene, triarylphosphine, oxanorbornadiene, diaryltetrazine, aryltetrazine, norbornene, aldehydes, hydroxylamine, hydrazine, NH₂—NH—C(═O)—, ketone, CoA or serine residue.

12. The antibody-drug conjugate of claim 10, wherein L_{I} is selected from the group consisting of p-aminobenzylcarbamoyl and dimethylethylenediamine.

13. The antibody-drug conjugate of claim 10, wherein each of the spacer is a PEG chain having 1 to 10 PEG units.

14. The antibody-drug conjugate of claim 10, wherein L_{C} is a linker comprising a peptide bond, a phosphate bond, a nucleic acid bond, a sugar bond, a disulfide bond, an amide bond, a substituted amide bond in the form of a peptide bond, a thioamide bond, an ester bond, a thioester bond, a vicinal diol bond, or a hemiacetal.

15. The antibody-drug conjugate of claim 10, wherein L_{M} is selected from the group consisting of —C(═O)—, C₁-C₁₈ alkylene group, —CH₂—CH═CH—, and phenylene group.

16. The antibody-drug conjugate of claim 8, wherein the antibody comprises a glycan or a modified glycan that is capable of undergoing a reaction with L_{R}.

17. The antibody-drug conjugate of claim 8, wherein the antibody is selected form the group consisting of an anti-HER2 antibody, an anti-EGFR antibody, an anti-PD-L1 antibody, an anti-VEGF antibody, an anti-HER3 antibody, an anti-TROP2 antibody, an anti-MET antibody, an anti-ROR1 antibody, an anti-ROR2 antibody, anti-BCMA antibody, anti-Mesothelin antibody, an anti-B7-H3 antibody, an anti-B7-H4 antibody, an anti-GPR20 antibody, an anti-tissue factor (TF) antibody, an anti-folate receptor α (FRα) antibody, an anti-Nectin-4 antibody, an anti-Somatostatin receptor 2 (SSTR2) antibody, anti-SSTR5 antibody, an anti-Claudin 18.2 antibody, an anti-LIV-1 antibody, an anti-Prostate-specific membrane antigen (PSMA) antibody, an anti-AXL antibody, an anti-CEACAM5 antibody, an anti-IGFIR antibody, an anti-EPHA2 antibody, an anti-MUC1 antibody, an anti-KIT antibody, an anti-DLL3 antibody, an anti-NaPi-2b antibody, an anti-MSLN antibody, an anti-5T4 antibody, an anti-CDH6 antibody, an anti-CDH7 antibody, an anti-CD37 antibody, an anti-CD30 antibody, and an anti-CD20 antibody, an anti-CD19 antibody, an anti-CD22 antibody, an anti-CD33 antibody, an anti-CD45 antibody, an anti-CD70 antibody, an anti-CD79B antibody, an anti-CD142 antibody.
